SEQUENCE LISTING (3 sequences)

SEQ ID NO: 1 - protein, 9 aa, Artificial Sequence
  Lyp-1 targeting peptide
CGNKRTRGC

SEQ ID NO: 2 - protein, 11 aa, Artificial Sequence
  targeting peptide
KLWVLPKGGGC

SEQ ID NO: 3 - protein, 7 aa, Artificial Sequence
  targeting peptide
KLWVLPK

(19) United States
(12) Patent Application Publication
(10) Pub. No.: US 2024/0016940 A1
(43) Pub. Date: Jan. 18, 2024

(54) LONG-ACTING AND LONG-CIRCULATING DELIVERY VEHICLES

(71) Applicant: The Brigham and Women`s Hospital, Inc., Boston, MA (US)

(72) Inventors: Jinjun Shi, Boston, MA (US); Yuling Xiao, Brookline, MA (US)

(21) Appl. No.: 18/038,392

(22) PCT Filed: Nov. 24, 2021
(86) PCT No.: PCT/US2021/060824

§ 371 Date: May 23, 2023

Related U.S. Application Data
(60) Provisional application No. 63/154,883, filed on Mar. 1, 2021.
(60) Provisional application No. 63/117,539, filed on Nov. 24, 2020.

(51) Int. Cl. A61K 47/54; A61K 47/69; A61K 48/00

(52) U.S. Cl. CPC A61K 47/543; A61K 48/005; A61K 48/0041; A61K 47/6937

(57) ABSTRACT

Described herein are novel compositions comprising lipid-poly(ethylene glycol) (lipid-PEG) molecules or lipid-PEG like molecules, and methods of use thereof, e.g., for sustained delivery of therapeutic agents such as nucleic acids, proteins, and small molecules.

### CLAIM OF PRIORITY

This application claims the benefit of U.S. Provisional Application Ser. No. 63/117,539, filed on Nov. 24, 2020, and 63/154,883, filed on Mar. 1, 2021. The entire contents of the foregoing are incorporated herein by reference.

### FEDERALLY SPONSORED RESEARCH OR DEVELOPMENT

This invention was made with Government support under Grant No. CA200900 awarded by the National Institutes of Health. The Government has certain rights in the invention.

### TECHNICAL FIELD

Described herein are novel compositions comprising lipid-poly(ethylene glycol) (lipid-PEG) molecules or lipid-PEG-like molecules, collectively also referred to herein as lipid-PEG or Cn-X-PEG, and methods of use thereof, e.g., for sustained delivery of therapeutic agents such as nucleic acids, proteins, and small molecules.

### BACKGROUND

Gene therapy that enables long-term production of endogenous functional proteins has been extensively pursued to address many unmet medical needs such as genetic disorders and cancer. For example, several adeno-associated virus (AAV)-based candidates are now in late-stage clinical trials for hemophilia A and hemophilia B^{1,2}. Despite encouraging short-term clinical results, this therapy also has concerns, such as significant inter-patient variability in FVIII or FIX production, potential for dilution of the functional gene as liver cells divide and undergo apoptosis, and potential unintended insertional mutagenesis¹. In addition, the pre-existing neutralizing AAV antibodies limit both the eligible population for initial dosing as well as the possibility of re-dosing.

Compared with AAV gene therapy, mRNA therapy has several advantages³⁻⁶: i) it does not require nuclear entry for transfection activity, and thus has a negligible chance of integrating into the host genome; ii) it has faster and more predictable protein expression; iii) the use of modified mRNA and non-viral delivery vehicles (e.g., lipid nanoparticles or LNPs) can largely avoid immune responses; iv) it can thus be re-dosed and provides a sustainable, longitudinal treatment option; v) it may be less sensitive to comorbidities and could be eligible to larger population; and vi) the costs will be much more affordable. Nanotechnology has shown promise to improve delivery of mRNA and other nucleic acids (e.g., siRNA)^{6,7}. For example, LNPs have been successfully used as the carrier for the first siRNA drug (Patisiran) and the two recently approved COVID-19 mRNA vaccines (BNT162b2 and mRNA-1273).

Despite these successes, one unique challenge associated with mRNA therapy and other RNA therapies is dealing with the transient activity due to their relatively short half-lives^{6,8,9}, therefore generally requiring frequent repeated dosing to sustain therapeutic effects. The diverse nanoparticle (NP) platforms previously reported have shown the capability to improve mRNA transfection efficiency; however, these mRNA NPs (including LNPs)-mediated protein expression usually reaches a peak rapidly and the duration is mainly limited to ˜2-7 days (also depending on cell type, tissue, animal model, and protein turnover, Table 1). This highlights the need for delivery platforms that can achieve long durable activity of mRNA and other RNAs.

### SUMMARY

The current invention describes novel lipid-PEG or lipid-PEG-like molecules (referred to collectively herein as lipid-PEG or Cn-X-PEG) for development of delivery vehicles that can achieve long-lasting RNA activity. In addition, such delivery vehicles comprising the novel lipid-PEG or lipid-PEG-like molecules have long systemic circulation lives and can be used for sustained delivery of various therapeutic agents (such as other nucleic acids, proteins, small molecules, and viruses) and imaging agents.

Provided herein are compounds of Formula (I):

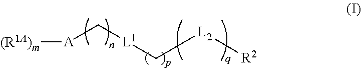

wherein:
A is selected from C₆₋₁₀ aryl and 5- to 10-membered heteroaryl, wherein the C₆₋₁₀ aryl or 5- to 10-membered heteroaryl is optionally substituted with one or more substituents independently selected from the group consisting of C₁₋₁₅ alkyl, C₂₋₁₅ alkenyl, C₂₋₁₅ alkynyl, C₃₋₁₀ cycloalkyl, C₆₋₁₀ aryl, 5- to 10-membered heteroaryl, 4- to 10-membered heterocycloalkyl, halo, —CN, —OR^{O}, —N(R^{N})₂, —(C═O)R^{N}, —(C═O)OR^{O}, —(C═O)N(R^{N})₂, and —NR^{N}(C═O)R⁸;
each R^{1A} is selected from C₁₋₁₀₀ alkyl, C₁₋₁₀₀ alkenyl, and C₁₋₁₀₀ alkynyl, and C₁₋₁₀₀ haloalkyl, wherein the C₁₃₋₁₀₀ alkyl, C₁₃₋₁₀₀ alkenyl, and C₁₃₋₁₀₀ alkynyl forming R¹ is optionally substituted with one or more substituents independently selected from the group consisting of halo, —CN, —OR^{O}, —N(R^{N})₂, —(C═O)R^{N}, —(C═O)OR^{O}, —(C═O)N(R^{N})₂, —NR^{N}(C═O)R⁸, and —O(C═O)R⁸;
L¹ is selected from bond, —N(R^{N})—, —O—, —(C═O)—, —(C═O)O—, —(C═O)N(R^{N})—, —NR^{N}(C═O)—, and —O(C═O)—;
L² is selected from:

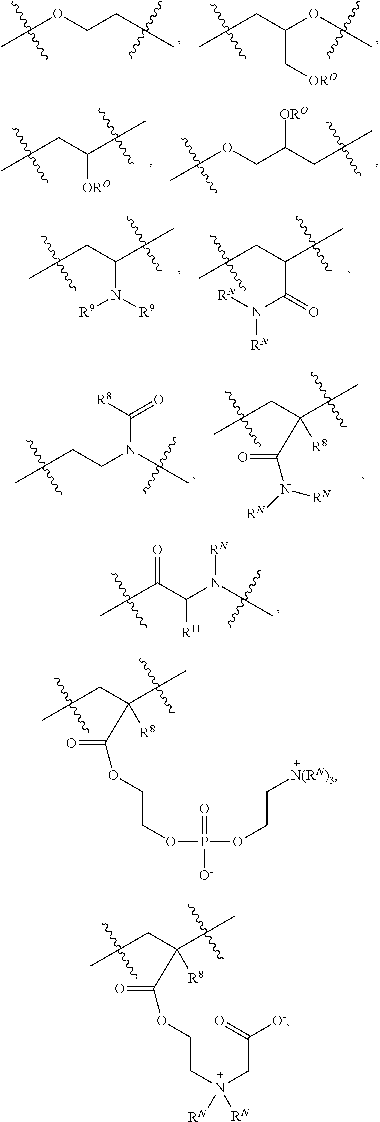

heparin, dextran, and chitosan;
R² is selected from H, C₁₋₁₅ alkyl, C₂₋₁₅ alkenyl, C₂₋₁₅ alkynyl, —OR^{O}, —(C═O)OR^{O}, —N(R^{N})₂, —N₃,

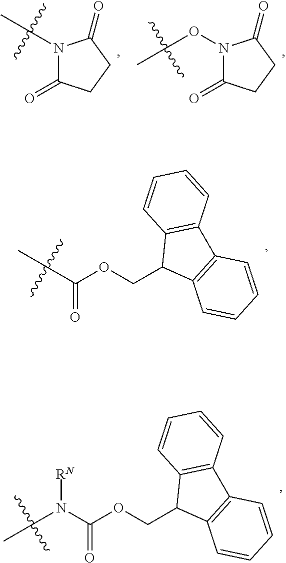

and targeting ligand;
each R⁸ is independently selected from H, C₁₋₁₅ alkyl, C₂₋₁₅ alkenyl, and C₂₋₁₅ alkynyl;
each R⁹ is independently selected from H, C₁₋₁₅ alkyl, C₂₋₁₅ alkenyl, and C₂₋₁₅ alkynyl;
or two R⁹, together with the N atom to which they are attached, come together to form 4- to 10-membered heterocycloalkyl optionally substituted with one or more oxo;
each R¹¹ is independently selected from C₁₋₁₅ alkyl, C₂₋₁₅ alkenyl, and C₂₋₁₅ alkynyl, optionally substituted with one or more R¹²;
each R¹² is independently selected from C₃₋₁₀ cycloalkyl, C₆₋₁₀ aryl, 5- to 10-membered heteroaryl, 4- to 10-membered heterocycloalkyl, halo, —CN, —OR^{O}, —N(R^{N})₂, —(C═O)R^{N}, —(C═O)OR^{O}, —(C═O)N(R^{N})₂, —NR^{N}(C═O)R⁸, —NR^{N}(C═NR^{N})R^{N}, —O(C═O)R⁸, and —SR⁸, wherein the C₃₋₁₀ cycloalkyl, C₆₋₁₀ aryl, 5- to 10-membered heteroaryl, 4- to 10-membered heterocycloalkyl is optionally substituted with one or more C₁₋₁₅ alkyl, C₂₋₁₅ alkenyl, and C₂₋₁₅ alkynyl, halo, —CN, —OR^{O}, and —N(R^{N})₂;
each R^{N} is independently selected from H, C₁₋₁₅ alkyl, C₂₋₁₅ alkenyl, and C₂₋₁₅ alkynyl, wherein the C₁₋₁₅ alkyl, C₂₋₁₅ alkenyl, and C₂₋₁₅ alkynyl forming R^{N} is optionally substituted with one or more substituents independently selected from the group consisting of halo, —CN, and —OR^{O};
each R^{O} is independently selected from H, C₁₋₁₅ alkyl, C₂₋₁₅ alkenyl, and C₂₋₁₅ alkynyl, wherein the C₁₋₁₅ alkyl, C₂₋₁₅ alkenyl, and C₂₋₁₅ alkynyl forming R^{O} is optionally substituted with one or more substituents independently selected from the group consisting of halo and —CN;
m is an integer selected from 1, 2, 3, 4, and 5;
n and p are each an integer independently selected from 0, 1, 2, 3, and 4; and
q is an integer selected from 1 to 2500;
provided that when A is phenyl, then each R^{1A} is selected from C₁₃₋₁₀₀ alkyl, C₁₋₁₀₀ alkenyl, C₁₋₁₀₀ alkynyl, and C₁₋₁₀₀ haloalkyl, wherein the C₁₃₋₁₀₀ alkyl, C₁₋₁₀₀ alkenyl, and C₁₋₁₀₀ alkynyl forming R¹ is optionally substituted with one or more substituents independently selected from the group consisting of halo, —CN, —OR^{O}, —N(R^{N})₂, —(C═O)R^{N}, —(C═O)OR^{O}, —(C═O)N(R^{N})₂, —NR^{N}(C═O)R⁸, and —O(C═O)R⁸.

In some embodiments, the compound is selected from:

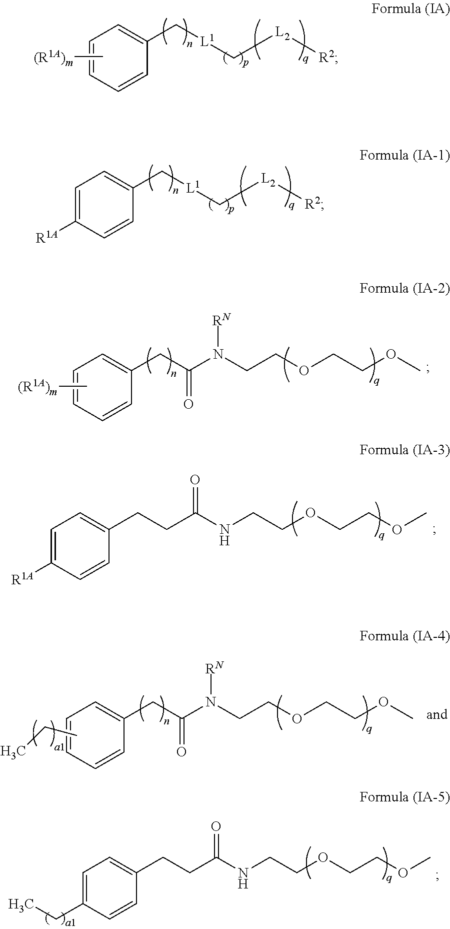

wherein a1 is an integer selected from 12 to 100.

In some embodiments, the compound is of Formula (I-1):

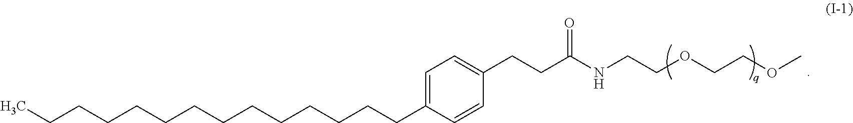

In some embodiments, the compound is a compound of Formula (I); A is C₆₋₁₀ aryl; each R^{1A} is C₁₃₋₁₀₀ alkyl; L¹ is —(C═O)N(R^{N})—; and L² is

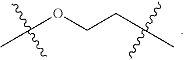 [SMILES: *CCO*]

In some embodiments, the compound is a compound of Formula (I) and A is C₆₋₁₀ aryl.

In some embodiments, the compound is a compound of Formula (I) and A is phenyl.

In some embodiments, the compound is a compound of Formula (I) and at least one R^{1A} is C₁₃₋₁₀₀ alkyl.

In some embodiments, the compound is a compound of Formula (I) and at least one R^{1A} is C₁₃₋₄₀ alkyl.

In some embodiments, the compound is a compound of Formula (I) and at least one R^{1A} is C₁₃₋₂₀ alkyl.

In some embodiments, the compound is a compound of Formula (I) and at least one R^{1A} is C₁₄ alkyl.

In some embodiments, the compound is a compound of Formula (I) and at least one R^{1A} is C₁₃₋₁₀₀ alkenyl.

In some embodiments, the compound is a compound of Formula (I) and at least one R^{1A} is C₁₃₋₁₀₀ alkynyl.

In some embodiments, the compound is a compound of Formula (I) and L¹ is (C═O)NH—.

In some embodiments, the compound is a compound of Formula (I) and L² is selected from

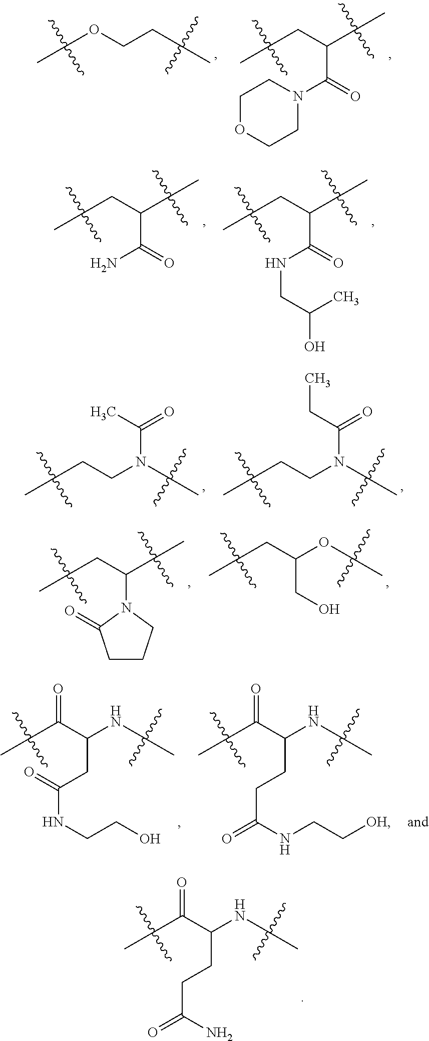

In some embodiments, the compound is a compound of Formula (I) and L² is

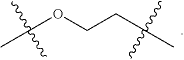 [SMILES: *CCO*]

In some embodiments, the compound is a compound of Formula (I) and R² is H.

In some embodiments, the compound is a compound of Formula (I) and R² is C₁₋₁₅ alkyl.

In some embodiments, the compound is a compound of Formula (I) and R² is —OR^{O}.

In some embodiments, the compound is a compound of Formula (I) and R² is —OCH₃.

In some embodiments, the compound is a compound of Formula (I) and R² is a targeting ligand.

In some embodiments, the targeting ligand is selected from a protein, a monosaccharide, a polysaccharide, a peptide, an aptamer, a small molecule, and a nucleic acid-based ligand.

In some embodiments, wherein the targeting ligand is selected from galactose and N-acetylgalactosamine (GalNAc).

In some embodiments, the compound is a compound of Formula (I) and m is 1.

In some embodiments, the compound is a compound of Formula (I) and n is 2.

In some embodiments, the compound is a compound of Formula (I) and p is 2.

Also provided herein are compounds of Formula (II):

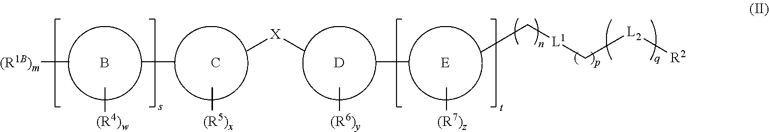

wherein:
X is selected from C(R³)₂, NR³, O, S, and

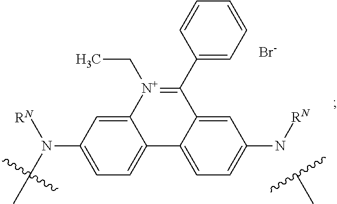

each R^{1B} is selected from C₁₋₁₀₀ alkyl, C₂₋₁₀₀ alkenyl, C₂₋₁₀₀ alkynyl, and C₁₋₁₀₀ haloalkyl, wherein the C₁₋₁₀₀ alkyl, C₁₋₁₀₀ alkenyl, and C₂₋₁₀₀ alkynyl forming R¹ is optionally substituted with one or more substituents independently selected from the group consisting of halo, —CN, —OR^{O}, —N(R^{N})₂, —(C═O)R^{N}, —(C═O)OR^{O}, —(C═O)N(R^{N})₂, —NR^{N}(C═O)R⁸, and —O(C═O)R⁸;
L¹ is selected from bond, —N(R^{N})—, —O—, —(C═O)—, —(C═O)O—, —(C═O)N(R^{N})—, —NR^{N}(C═O)—, and —O(C═O)—;
L² is selected from:

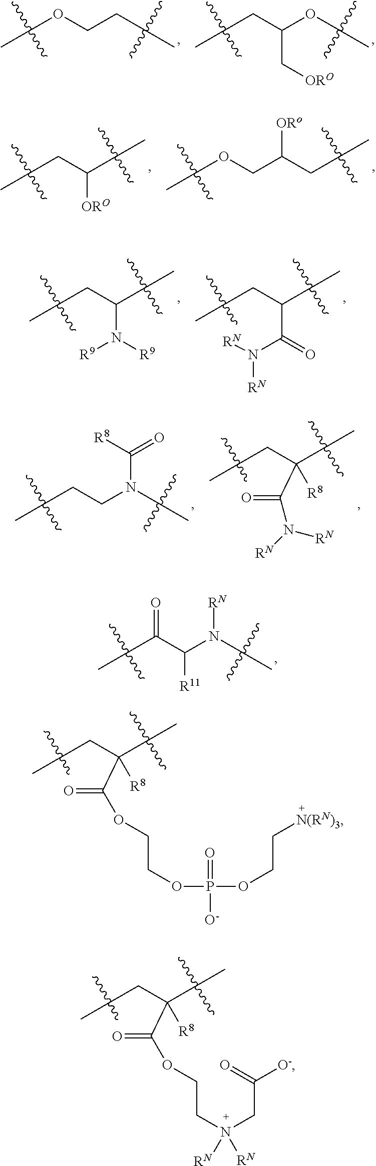

heparin, dextran, and chitosan;
   R² is selected from H, C₁₋₁₅ alkyl, C₂₋₁₅ alkenyl, C₂₋₁₅ alkynyl, —OR^{O}, —(C═O)OR^{O}, —N(R^{N})₂, —N₃,

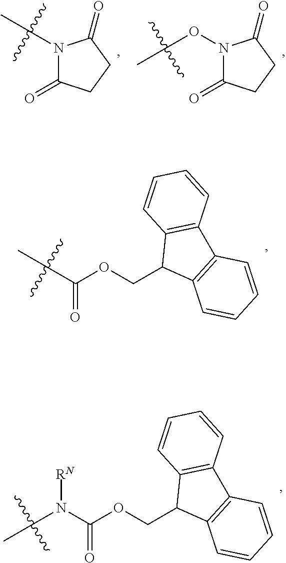

and targeting ligand;
each R³ is independently selected from H, C₁₋₁₅ alkyl, C₂₋₁₅ alkenyl, and C₂₋₁₅ alkynyl, C₃₋₁₀ cycloalkyl, C₆₋₁₀ aryl, 5- to 10-membered heteroaryl, and 4- to 10-membered heterocycloalkyl optionally substituted with one or more R¹⁰;
each Ring B, Ring C, Ring D, and Ring E is independently selected from C₆₋₁₀ aryl or 5- to 10-membered heteroaryl;
each R⁴, R⁵, R⁶, and R⁷ is independently selected from C₁₋₁₅ alkyl, C₂₋₁₅ alkenyl, C₂₋₁₅ alkynyl, C₃₋₁₀ cycloalkyl, C₆₋₁₀ aryl, 5- to 10-membered heteroaryl, 4- to 10-membered heterocycloalkyl, halo, —CN, —OR^{O}, —N(R^{N})₂, —(C═O)R^{N}, —(C═O)OR^{O}, —(C═O)N(R^{N})₂, —NR^{N}(C═O)R⁸, and —O(C═O)R⁸;
or an R⁵ and an R⁶, together with the atoms to which they are attached, come together to form C₆₋₁₀ aryl or 5- to 10-membered heteroaryl, wherein the C₆₋₁₀ aryl or 5- to 10-membered heteroaryl is optionally substituted with one or more R⁸;
each R⁸ is independently selected from H, C₁₋₁₅ alkyl, C₂₋₁₅ alkenyl, and C₂₋₁₅ alkynyl;
each R⁹ is independently selected from H, C₁₋₁₅ alkyl, C₂₋₁₅ alkenyl, and C₂₋₁₅ alkynyl;
or two R⁹, together with the N atom to which they are attached, come together to form 4- to 10-membered heterocycloalkyl optionally substituted with one or more oxo;
each R¹⁰ is independently selected from the group consisting of C₁₋₁₀₀ alkyl, C₂₋₁₀₀ alkenyl, C₂₋₁₀₀ alkynyl, C₁₋₁₀₀ haloalkyl, halo, —CN, —OR^{O}, oxo, —N(R^{N})₂, —(C═O)R^{N}, —(C═O)OR^{O}, —(C═O)N(R^{N})₂, —NR^{N}(C═O)R⁸, and —O(C═O)R⁸;
or an R⁵ and an R¹⁰, together with the atoms to which they are attached, come together to form C₆₋₁₀ aryl or 5- to 10-membered heteroaryl, wherein the C₆₋₁₀ aryl or 5- to 10-membered heteroaryl is optionally substituted with one or more R⁸;
or an R⁶ and an R¹⁰, together with the atoms to which they are attached, come together to form C₆₋₁₀ aryl or 5- to 10-membered heteroaryl, wherein the C₆₋₁₀ aryl or 5- to 10-membered heteroaryl is optionally substituted with one or more R⁸;
each R¹¹ is independently selected from C₁₋₁₅ alkyl, C₂₋₁₅ alkenyl, and C₂₋₁₅ alkynyl, optionally substituted with one or more R¹²;
each R¹² is independently selected from C₃₋₁₀ cycloalkyl, C₆₋₁₀ aryl, 5- to 10-membered heteroaryl, 4- to 10-membered heterocycloalkyl, halo, —CN, —OR^{O}, —N(R^{N})₂, —(C═O)R^{N}, —(C═O)OR^{O}, —(C═O)N(R^{N})₂, —NR^{N}(C═O)R⁸, —NR^{N}(C═NR^{N})R^{N}, —O(C═O)R⁸, and —SR⁸, wherein the C₃₋₁₀ cycloalkyl, C₆₋₁₀ aryl, 5- to 10-membered heteroaryl, 4- to 10-membered heterocycloalkyl is optionally substituted with one or more C₁₋₁₅ alkyl, C₂₋₁₅ alkenyl, and C₂₋₁₅ alkynyl, halo, —CN, —OR^{O}, and —N(R^{N})₂;
each R^{N} is independently selected from H, C₁₋₁₅ alkyl, C₂₋₁₅ alkenyl, and C₂₋₁₅ alkynyl, wherein the C₁₋₁₅ alkyl, C₂₋₁₅ alkenyl, and C₂₋₁₅ alkynyl forming R^{N} is optionally substituted with one or more substituents independently selected from the group consisting of halo, —CN, and —OR^{O};
each R^{O} is independently selected from H, C₁₋₁₅ alkyl, C₂₋₁₅ alkenyl, and C₂₋₁₅ alkynyl, wherein the C₁₋₁₅ alkyl, C₂₋₁₅ alkenyl, and C₂₋₁₅ alkynyl forming R^{O} is optionally substituted with one or more substituents independently selected from the group consisting of halo and —CN;
m is an integer selected from 1, 2, 3, 4, and 5;
n and p are each an integer independently selected from 0, 1, 2, 3, and 4;
q is an integer selected from 1 to 2,500;
s and t are each an integer independently selected from 0, 1, 2, 3, 4, and 5; and
w, x, y, and z are each an integer independently selected from 0, 1, 2, 3, and 4.
   In some embodiments, the compound is selected from:

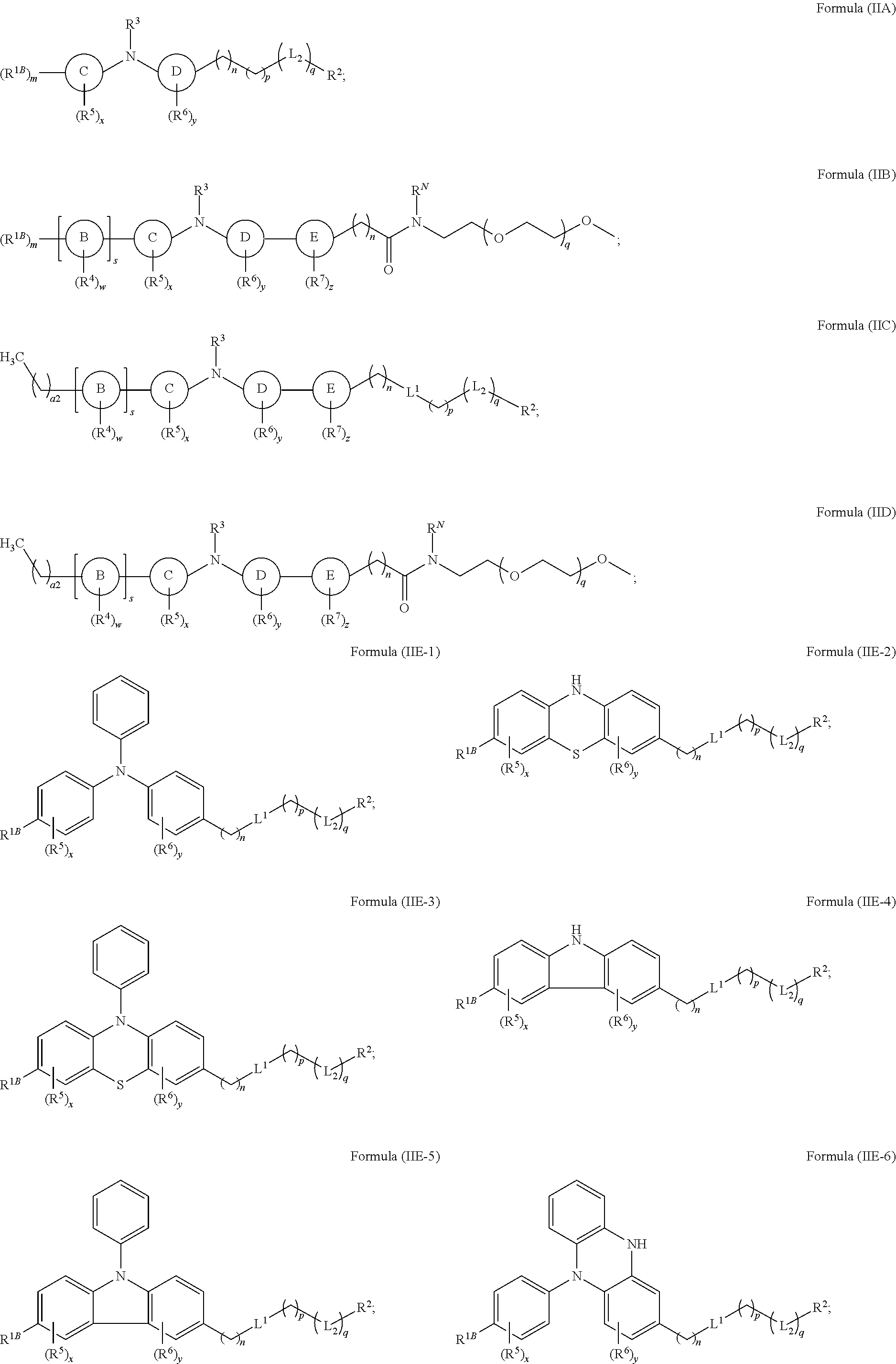

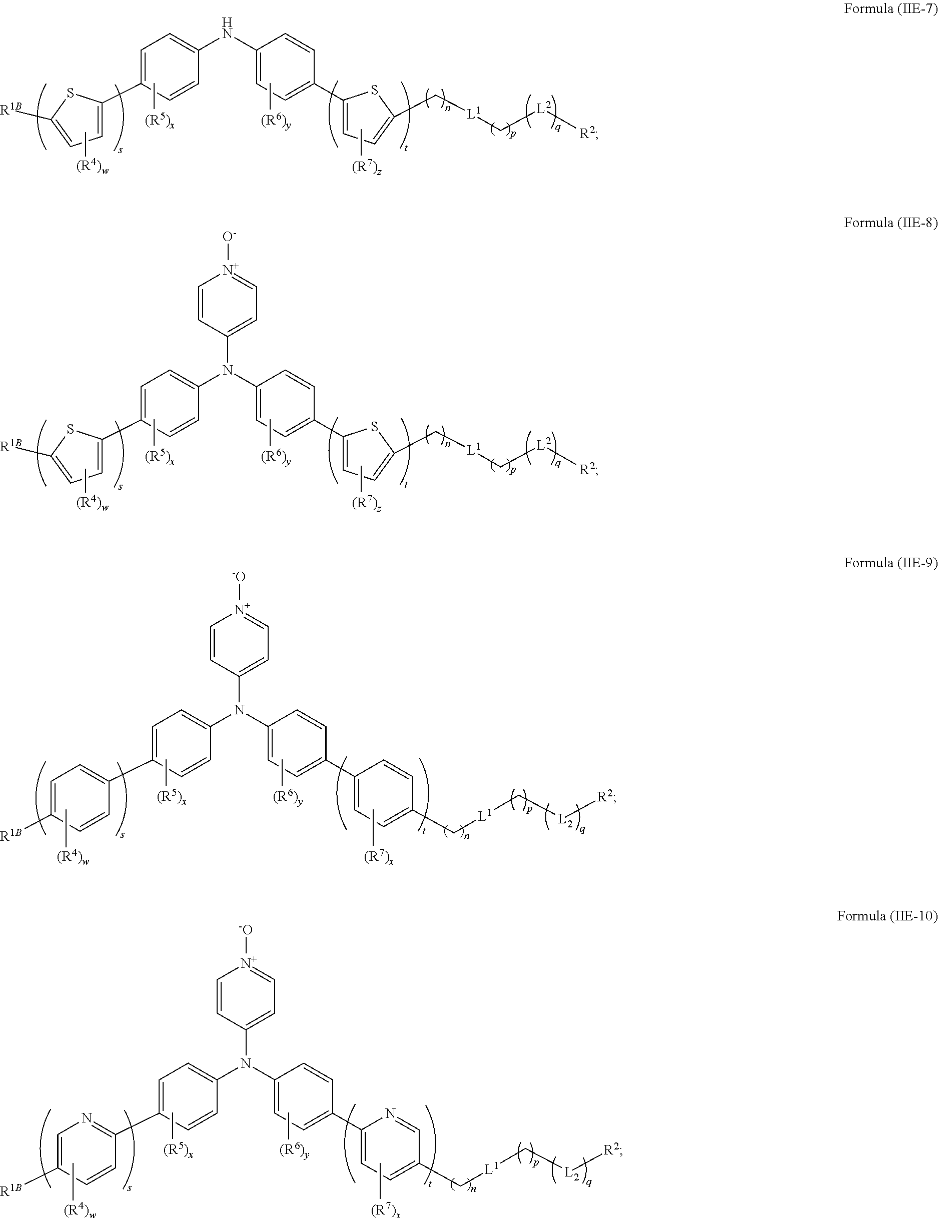

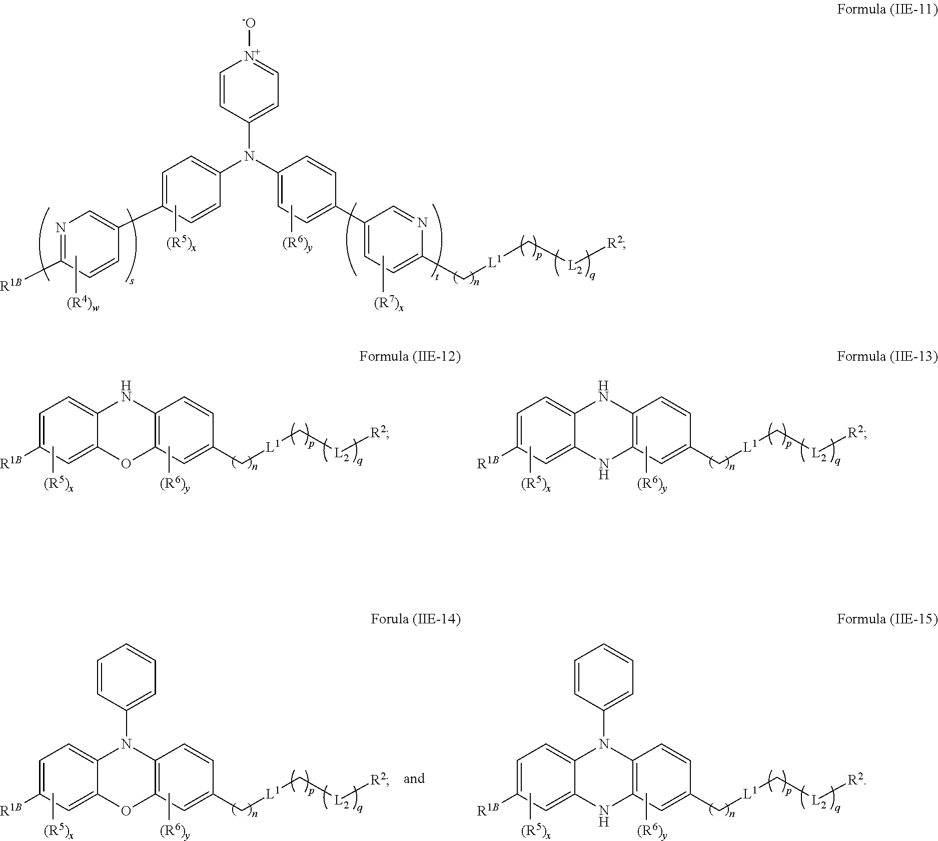

The compound of claim 26, wherein the compound is selected from:

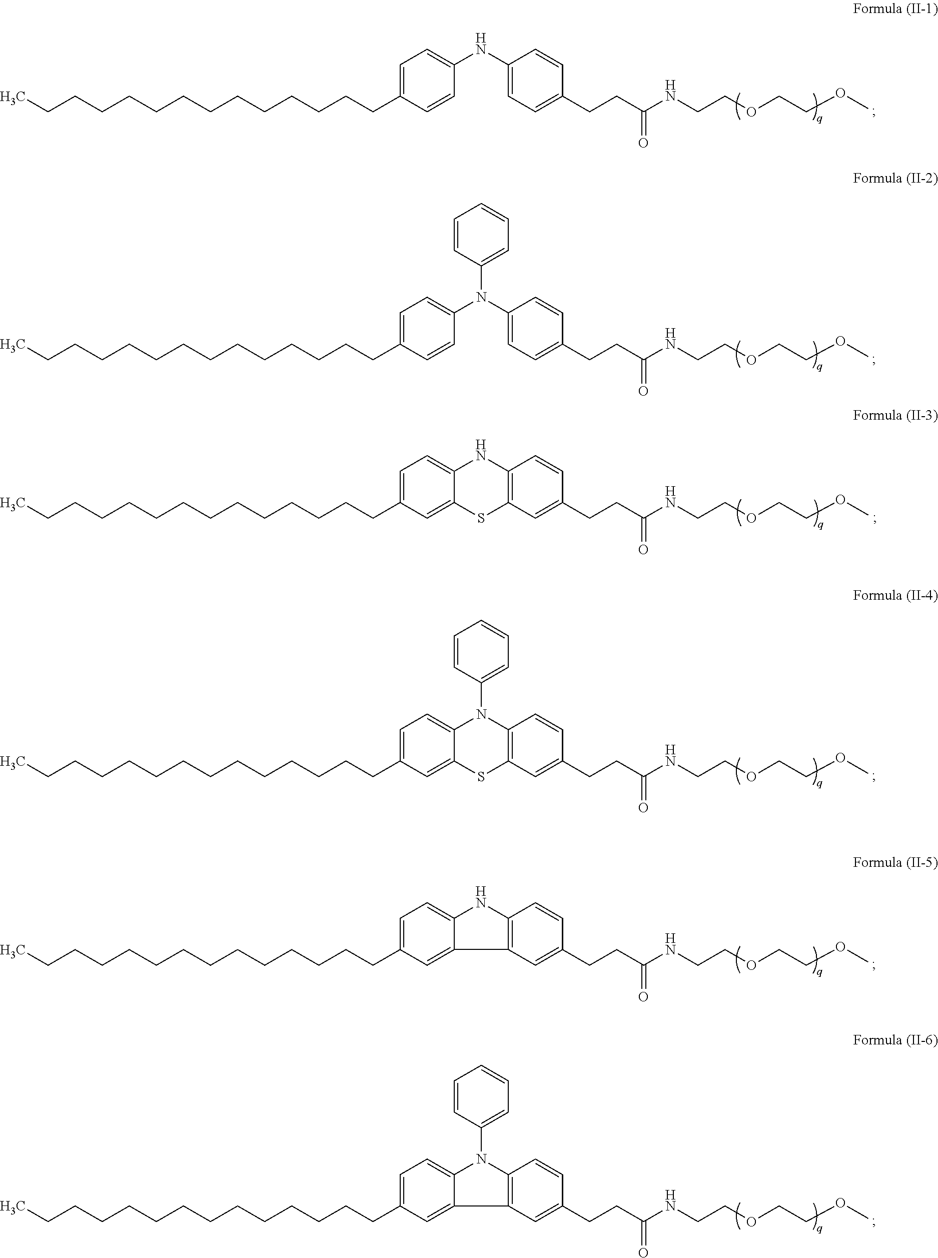

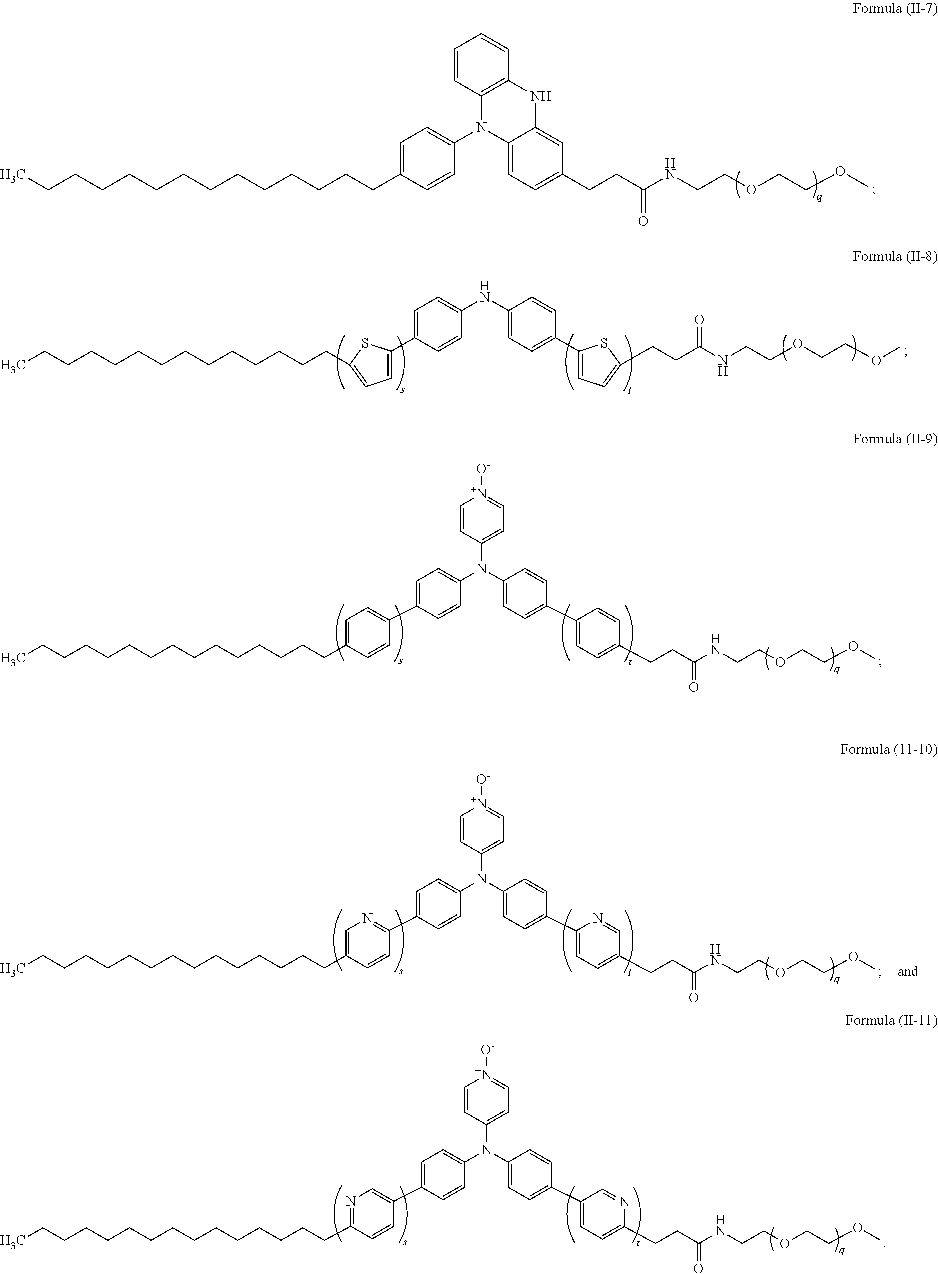

In some embodiments, the compound is a compound of Formula (II); each R^{1B} is C₁₋₁₀₀ alkyl; L¹ is —(C═O)N(R^{N})—; L² is

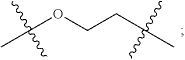 [SMILES: *CCO*]

R² is C₁₋₁₅ alkyl; R³ is selected from H and C₆₋₁₀ aryl; each R^{N} is H; and each R^{O} is C₁₋₁₅ alkyl.

In some embodiments, the compound is a compound of Formula (II) and at least one R^{1B} is C₁₋₁₀₀ alkyl.

In some embodiments, the compound is a compound of Formula (II) and at least one R^{1B} is C₁₋₄₀ alkyl.

In some embodiments, the compound is a compound of Formula (II) and at least one R^{1B} is C₁₃₋₂₀ alkyl.

In some embodiments, the compound is a compound of Formula (II) and at least one R^{1B} is C₁₄ alkyl.

In some embodiments, the compound is a compound of Formula (II) and L¹ is —(C═O)NH—.

In some embodiments, the compound is a compound of Formula (I) and L² is selected from

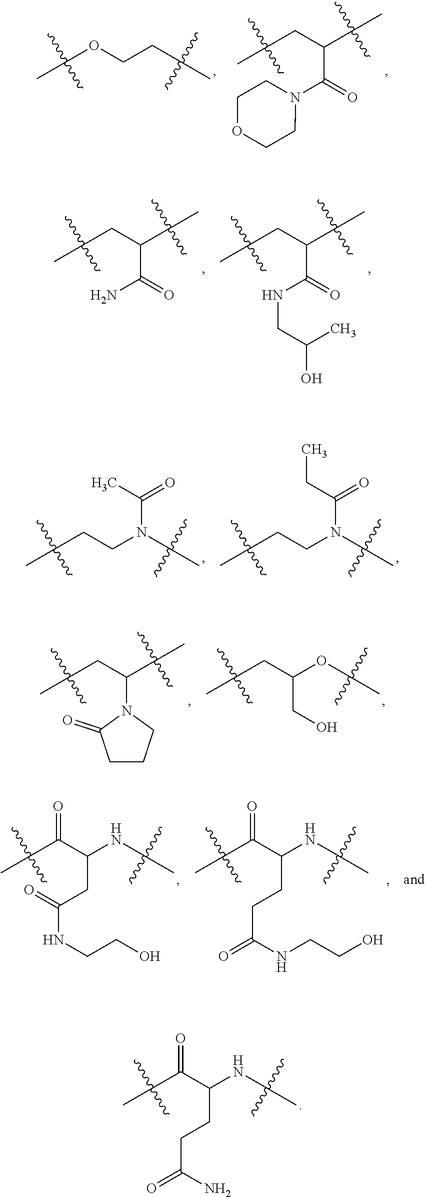

In some embodiments, the compound is a compound of Formula (II) and L² is

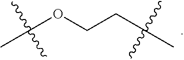 [SMILES: *CCO*]

In some embodiments, the compound is a compound of Formula (II) and R² is H.

In some embodiments, the compound is a compound of Formula (II) and R² is C₁₋₁₅ alkyl.

In some embodiments, the compound is a compound of Formula (II) and R² is —OR^{O}.

In some embodiments, the compound is a compound of Formula (II) and R² is a targeting ligand.

In some embodiments, the targeting ligand is selected from a protein, a monosaccharide, a polysaccharide, a peptide, an aptamer, a small molecule, and a nucleic acid-based ligand.

In some embodiments, the targeting ligand is selected from galactose and N-acetylgalactosamine (GalNAc).

In some embodiments, the compound is a compound of Formula (II) and R³ is H.

In some embodiments, the compound is a compound of Formula (II) and R³ is C₆₋₁₀ aryl.

In some embodiments, the compound is a compound of Formula (II) and R³ is 5- to 10-membered heteroaryl.

In some embodiments, the compound is a compound of Formula (II) and R³ is selected from H, phenyl, pyridinyl,

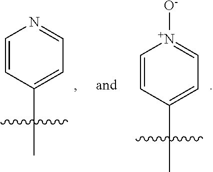 [SMILES: *c1ccncc1 ; *c1cc[n+]([O-])cc1]

In some embodiments, the compound is a compound of Formula (II) and at least one Ring B is 5- to 10-membered heteroaryl.

In some embodiments, the compound is a compound of Formula (II) and at least one Ring B is phenyl.

In some embodiments, the compound is a compound of Formula (II) and at least one Ring B is pyridinyl.

In some embodiments, the compound is a compound of Formula (II) and at least one Ring B is thiophenyl.

In some embodiments, the compound is a compound of Formula (II) and Ring C is C₆₋₁₀ aryl.

In some embodiments, the compound is a compound of Formula (II) and Ring C is phenyl.

In some embodiments, the compound is a compound of Formula (II) and Ring D is C₆₋₁₀ aryl.

In some embodiments, the compound is a compound of Formula (II) and Ring D is phenyl.

In some embodiments, the compound is a compound of Formula (II) and at least one Ring E is 5- to 10-membered heteroaryl.

In some embodiments, the compound is a compound of Formula (II) and at least one Ring E is phenyl.

In some embodiments, the compound is a compound of Formula (II) and at least one Ring E is pyridinyl.

In some embodiments, the compound is a compound of Formula (II) and at least one Ring E is thiophenyl.

In some embodiments, the compound is a compound of Formula (II) and m is 1.

In some embodiments, the compound is a compound of Formula (II) and n is 2.

In some embodiments, the compound is a compound of Formula (II) and p is 2.

Also provided herein are compositions comprising a compound as described herein, optionally wherein the compound of Formula (I) or (II).

In some embodiments, the composition is a particle, e.g., a nanoparticle, optionally a liposome.

In some embodiments, the composition further comprises one or more additional lipids. In some embodiments, the additional lipids comprise: one or more ionizable lipids, optionally selected from G0-Cm, DLin-MC3-DMA ((6Z,9Z,28Z,31Z)-heptatriacont-6,9,28,31-tetraene-19-yl 4-(dimethylamino)butanoate), SM-102, ALC-0315, and multi-tailed ionizable phospholipids (iPhos; one or more phospholipids selected from phosphatidylethanolamine (optionally DOPE) and phosphatidylcholine (optionally DSPC); one or more cholesterol and its analogues; and/or other lipids, optionally selected from dioleoyl-3-trimethylammonium propane (DOTAP), 1,2-di-O-octadecenyl-3-trimethylammonium propane (DOTMA), DDAB, DODAP, EPC, and 18BMP.

In some embodiments, the nanoparticle further comprises a cargo. In some embodiments, the cargo is presented on the surface of the nanoparticle, or the nanoparticle comprises a core and an envelope, wherein the core comprises a lipid and a cargo, optionally wherein the cargo is complexed with the lipid.

In some embodiments, the cargo comprises RNA, DNA, protein, or a small molecule. In some embodiments, the RNA or DNA encodes, or the protein comprises: a therapeutic protein, a tumor suppressor, an antigen, a cytokine, gene editing reagent, or a co-stimulatory molecule. In some embodiments, the therapeutic protein is listed in Table 2, 4, or 6. In some embodiments, the tumor suppressor is listed in Table 5. In some embodiments, the mRNA comprises one or more modifications, preferably selected from the group consisting of ARCA capping; enzymatic polyadenylation to add a tail of 100-250 adenosine residues; and substitution of one or both of cytidine with 5-methylcytidine and/or uridine with pseudouridine.

Additionally, provided herein are method of treating a subject who has cancer. The methods can include administering to the subject a therapeutically effective amount of a composition as described herein, wherein the RNA or DNA encodes, or the protein comprises, a tumor suppressor, an antigen, a cytokine, or a co-stimulatory molecule.

Further, provided herein are methods for treating a subject who has a genetic disorder. The methods can include administering to the subject a therapeutically effective amount of a composition as described herein, wherein the RNA or DNA encodes, or the protein comprises, a therapeutic for the genetic disorder.

Also provided herein are methods for subject who has hemophilia. The methods include administering to the subject a therapeutically effective amount of a composition as described herein, wherein the RNA or DNA encodes, or the protein comprises, Factor VIII or Factor IX.

Also provided herein are methods for treating a subject who has an infectious disease associated with an infectious agent, or reducing risk of developing an infectious disease with an infectious agent. The methods include administering to the subject a therapeutically effective amount of a composition as described herein, wherein the RNA or DNA encodes, or the protein comprises an antigen associated with the infectious agent.

Additionally provided herein are methods for administering a therapeutic agent to a subject. The methods include administering to the subject a therapeutically effective amount of the composition as described herein, wherein the cargo comprises the therapeutic agent, or comprises RNA or DNA that encodes, or a protein that comprises, the therapeutic agent. In some embodiments, the therapeutic agent is an antibody or a gene editing reagent.

Additionally, provided are lipid-PEG composition or lipid-PEG-like composition as shown herein or modified as set forth herein, as well as the use of the compositions to deliver therapeutic nucleic acids to a patient in need of the same. In some embodiments, the nucleic acid is mRNA. In some embodiments, the nucleic acid is circular RNA. In some embodiments, the nucleic acid is tRNA or its fragment. In some embodiments, the nucleic acid is siRNA or dsiRNA or microRNA or piwiRNA or antisense oligonucleotide. In other embodiments, the nucleic acid is a mixture of different types of RNA.

Also provided are methods for protein replacement. The methods include administering a composition including an mRNA encoding a protein in need of replacement encapsulated in a nanoparticle coated with a lipid-PEG composition as described herein, as well as methods of treating cancers or genetic diseases comprising administering a composition including mRNA encapsulated in a nanoparticle coated with a lipid-PEG composition as described herein.

Further, provided herein are methods for engineering cells by incubating cells ex vivo or directly in situ with a composition including mRNA encapsulated in a nanoparticle coated with a lipid-PEG composition as described herein.

In some embodiments, the methods can be used for engineering cells by delivering a gene engineering reagent as described here.

The compositions and methods can also be used to deliver an antigen (e.g., a protein or peptide antigen, or a nucleic acid encoding a protein or peptide antigen) to a subject to elicit an immune response to the antigen, e.g., for use as a vaccine. Preferably the antigen is from a pathogen or infectious agent (e.g., virus, bacteria, or fungus).

The present methods and compositions can also be used for systemic secretion of therapeutic proteins (such as antibodies) and peptides, and for delivery of reagents for gene silencing (e.g., inhibitory nucleic acids such as siRNA, ASOs, microRNA, and so on).

Also described herein are nucleic acid carrier systems comprising a lipid-PEG composition as described herein, wherein the carriers are nanoparticles. In some embodiments, the nanoparticles are hybrid polymer-lipid NPs or lipid nanoparticles (LNPs), liposomes, or polymeric NPs. In some embodiments, the nanoparticles are polymeric micelles or lipid micelles. In some embodiments, the nanoparticles are exosomes or extracellular vesicles. In some embodiments, the nanoparticles are cell membrane-derived vehicles. In some embodiments, the nanoparticles are nanogels. In some embodiments, the nanoparticles are viruses. In some embodiments, the nanoparticles are inorganic nanomaterials. In other embodiments, the carriers are microparticles or cells.

Additionally, provided herein are methods for treating a disease or condition in a subject comprising administering an effective amount of a composition as described herein or its combination with other therapies, wherein the disease or condition is selected from the group consisting of: cancer, a genetic blood disorder, genetic disorders that are characterized by protein deficiencies or malfunctions (such as thrombotic thrombocytopenic purpura, methylmalonic academia, hereditary tyrosinemia type 1, Fabry disease, acute intermittent porphyria, alpha-1 antitrypsin deficiency, glycogen storage disease type 1, cystic fibrosis, and others), pain, infectious diseases (e.g., a viral infection such as COVID-19), neurodegenerative diseases, diabetes, inflammatory diseases, metabolic diseases, cardiovascular diseases, cardiometabolic diseases, eye diseases, ear diseases, and others.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1. Chemical structures of exemplary lipid-PEGs (or lipid-PEG-like molecules): Cn-X-PEG. The schematic only shows the n number of 14, but it could be different, e.g., from 2 to 40. In addition, the schematic only shows one alkyl chain, but it could have two or more alkyl chains (either saturated or non-saturated), or even other hydrophobic tail(s). Moreover, the PEG molecule can be replaced with other hydrophilic polymers.

FIGS. 2A-B. Schematic of (a) lipid nanoparticle (LNP) platforms and (b) polymer-lipid hybrid mRNA NPs (HNPs) for RNA delivery. Note that the LNP platform could be composed of i) lipid-PEG, ionizable lipid, phospholipid, and cholesterol as shown in (a), or ii) lipid-PEG, phospholipid, and cholesterol, or iii) lipid-PEG and cholesterol, or iv) lipid-PEG and phospholipid, or v) lipid-PEG only. The phospholipid can be, e.g., a phosphatidylcholine (e.g., DSPC), phosphatidylethanolamine (e.g., DOPE), or other phospholipids. The cholesterol could be replaced with cholesterol derivatives. The lipid-PEG is a Cn-X-PEG such as C14-TPA-PEG. The lipid-PEG layer could also contain traditional lipid-PEG molecules such as Dimyristoyl glycerol (DMG)-PEG (C14-PEG) and DSPE-PEG. The ionizable lipid could be DLin-MC3-DMA (MC3) lipid, G0-C8, SM-102, ALC-0315, or others (see Chem. Rev. 2021, 121, 20, 12181-12277)¹⁰. In addition, the lipid-PEG can be conjugated with one or more targeting ligand(s) to improve specific delivery. The HNPs platform consists the ionizable lipid (e.g., lipid-like compound G0-C8) for mRNA complexation, the hydrophobic polymer [e.g., poly(lactic-co-glycolic acid) (PLGA)] for forming a stable NP core to carry the G0-C8/mRNA complexes, and the lipid-PEG (e.g., C14-TPA-PEG) layer for stability.

FIGS. 3A-D. Schematic and characterization of an exemplary mRNA LNP formulation. a. Schematic of a mRNA LNP. The lipid-PEG is C14-TPA-PEG. The ionizable lipid is MC3. The phospholipid is DOPE. b. Agarose gel electrophoresis assay of mRNA stability in DMF (Lane 1), PBS (Lane 2), in LNPs (Lane 3), lipid-polymer hybrid NPs (Lane 4), or in Lipofectamine 2000 (Lipo2k) (Lane 5). c. Average particle size and zeta potential of the EGFP mRNA LNPs, Luciferase mRNA LNPs, and PTEN mRNA LNPs (n=3). d. TEM image of EGFP mRNA LNPs. Scale bar, 200 nm.

FIGS. 4A-B. (a) Stability of mRNA LNPs in 10% serum condition at 37° C. was evaluated by measuring particle size changes at various time points up to 15 days. (b) In vitro toxicity of EGFP mRNA LNPs in PTEN-Cap8 cells at different mRNA concentrations (0.25, 0.5, 1, 2 or 5 μg/mL).

FIGS. 5A-C. In vitro cellular uptake of Cy5-Luc-mRNA LNPs in PTEN Cap-8 cells at different mRNA concentrations. (a) Control (without mRNA LNPs); (b) mRNA concentrations of 0.25 μg/mL, and (c) mRNA concentrations of 0.5 μg/mL.

FIGS. 6A-B. Schematic and characterization of a targeted mRNA LNPs: galactose-modified C14-TPA-PEG LNPs (Gal-C14-TPA-PEG LNPs). A. Schematic representation of the targeted mRNA LNP. B. Average particle size (left bar) and zeta potential (right bar) of the Gal-C14-TPA-PEG LNPs (n=3).

FIG. 7. Transfection efficiency and duration of luciferase-mRNA in THLE-3 and Hep3B cells by different LNPs: traditional MC3 LNPs (with C14-PEG); MC3 LNPs (with C14-TPA-PEG); and traditional LNPs with the ionizable lipid G0-C8 and C14-PEG. mRNA concentration is 0.25 μg/mL.

FIG. 8. Effect of lipid-PEG on GFP protein expression duration by EGFP-mRNA LNPs in THLE-3 cells (mRNA concentration: 0.25 μg/mL). NPs were incubated for 1 day and then washed and replaced with fresh medium. Cells were trypsinized every 3-4 days; 6,000 cells were then re-cultured in 96-well plate. As can be seen, the C14-TPA-PEG-coated LNPs can induce durable GFP expression for 34 days. In comparison, traditional C14-PEG-coated LNPs can only induce GFP expression for ˜6 days. Free mRNA doesn't induce protein expression. This study suggests that the mRNA LNPs described herein can induce long-term durable protein expression in cells, which will be highly impactful for many biomedical applications and could significantly reduce the dosing frequency of mRNA therapy.

FIG. 9. GFP expression in RAW264.7 macrophage cells after treatment with the LNPs (mRNA concentration: 0.25 μg/mL) for 1 day. As can be seen, the C14-TPA-PEG-coated LNPs can induce durable GFP expression in macrophage cells for >23 days. This study indicates that the mRNA LNPs could be used for long-term engineering of therapeutic cells including immune cells.

FIGS. 10A-B. Luciferase expression in C57BL/6 mice after a single intravenous (IV) injection of the luciferase mRNA LNPs at day 0. (A) Bioluminescence imaging by IVIS. (B) Quantification of luminescence intensity from (A) as a function of time. The IV injection dose of mRNA is 0.25 mg/kg. Note that the scale bar for day 0.2-7 and for day 9-15 is different. MC3 LNPs are coated with traditional DMG-PEG. C14-TPA-PEG LNPs are coated with the C14-TPA-PEG. Gal-C14-TPA-PEG LNPs are coated with galactose-conjugated C14-TPA-PEG. Both of the LNPs use the MC3 ionizable lipid for head-to-head comparison. This result shows that the mRNA LNPs induced a strong and stable luciferase expression for 7 days and that the expression lasted ˜12-14 days. In comparison, the traditional MC3 LNPs-mediated luciferase expression in the liver only lasted for ˜2 days. This study indicates that the mRNA LNPs could be used for long-term in vivo protein replacement or generation of therapeutic proteins.

FIG. 11. hEPO expression in the blood of C57BL/6 mice after a single IV treatment with the Gal-C14-TPA-PEG LNPs (mRNA dose: 0.25 mg/kg). The result shows a durable presence of hEPO in the blood for 14 days above 100 ng/mL and for 21 days above 7 ng/mL.

FIG. 12. Circulation profile of free Cy5-EGFP mRNA and two different mRNA LNP formulations with DSPE-PEG (termed as DSPE-PEG LNPs) or the C14-TPA-PEG (termed as C14-TPA-PEG LNPs) in normal BALB/C mice after IV injection through tail vein. Error bars represent the S.D. (n=3 mice per group for DSPE-PEG and C14-TPA-PEG LNPs; n=2 mice for free mRNA). This long blood circulation may be beneficial for more effective mRNA delivery to diseased tissues such as tumor, improving therapeutic efficacy and reducing dosing frequency.

FIG. 13. Hepatocellular carcinoma (HCC) cell viability after treatment with control EGFP-mRNA and p53-mRNA using the LNPs coated with C14-TPA-PEG: (top) RIL-175 cells; (bottom) Hep3B cells. The cells were incubated empty LNPs, control EGFP-mRNA LNPs, or p53-mRNA LNPs at three different concentrations (0.125, 0.25, and 0.5 Ng/mL) for 1 day, washed and further incubated with fresh medium for another day. The cell viability was measured by Alamarblue assay. As can be seen, treatment with p53-mRNA LNPs reduced cell viability in a dose-dependent manner. This study indicates the possibility of using mRNA LNPs for cancer treatment by themselves or in combination with other therapies such as chemotherapy, radiotherapy, immunotherapy including immune checkpoint blockade therapy, and phototherapy.

FIGS. 14A-B. Sustained cytotoxicity of PTEN mRNA NPs. (a) Effect of lipid-PEGS on anti-tumor effect in PTEN-Cap8 cells treated by PTEN mRNA polymer-lipid hybrid NPs. (b) Effect of lipid-PEGS on anti-tumor effect in PTEN-Cap8 cells treated by PTEN mRNA LNPs. The PTEN mRNA concentration is 0.25, 0.5, or 0.75 μg/mL, and the NPs were incubated with cells for 24 h and washed away with fresh medium.

FIG. 15. Effect of the density of lipid-PEG on GFP transfection efficiency by EGFP-mRNA LNPs coated with C14-TPA-PEG in THLE-3 cells (mRNA concentration: 0.25 μg/mL). NPs were incubated for 1 day and then washed and replaced with fresh medium. Images were taken at day 2.

FIGS. 16A-B. Effect of different C14-n-PEGs (16A) on GFP transfection efficiency (16B) by EGFP-mRNA LNPs in THLE-3 cells (mRNA concentration: 0.25 μg/mL). NPs were incubated for 1 day and then washed and replaced with fresh medium. D-Lin in FIG. 16B is the same, with MC3 ionizable lipid.

FIG. 17. Effect of alkyl chain lengths in the lipid-PEG on GFP protein expression duration by EGFP-mRNA LNPs in THLE-3 cells (mRNA concentration: 0.25 μg/mL). NPs were incubated for 1 day and then washed and replaced with fresh medium. Cells were trypsinized every 3-4 days; 6,000 cells were then re-cultured in 96-well plate.

FIG. 18. In vivo toxicity studies: hematological analysis based on serum biochemistry with the mRNA LNPs formulated with Cn-TPA-PEGs. The mRNA concentration is 0.25 mg/kg for #1 to #5, 1 mg/kg for #6, and 2 mg/kg for #7.

1: C10-TPA-PEG Luc LNPs

2: C12-TPA-PEG Luc LNPs

3: C14-TPA-PEG Luc LNPs

4: C16-TPA-PEG Luc LNPs

5: C18-TPA-PEG Luc LNPs

6: C14-TPA-PEG Luc LNPs 1 mg/kg

7: C14-TPA-PEG Luc LNPs 2 mg/kg

C: saline

FIGS. 19A-B. Characterization of C14-TPA-PEG/EGFP mRNA mixture. (a) Average particle size and zeta potential. (b) The stability of C14-TPA-PEG/EGFP mRNA complexes in 10% serum at 37° C. Lane 1: free mRNA in DMF at day 0; Lane 2: DSPE-PEG/EGFP mRNA complexes at day 0; Lanes 3-5: C14-TPA-PEG/EGFP mRNA complexes at day 0, 2 and day 4.

FIG. 20. GFP protein expression in THLE-3 cells by EGFP-mRNA/C14-TPA-PEG complexes at different time points (mRNA concentration: 0.25 μg/mL). The results in FIGS. 19 and 20 indicate that C14-TPA-PEG could stabilize mRNA, contributing to the long-durable mRNA activity.

FIG. 21. Silencing efficacy and duration of luciferase-siRNA hybrid NPs (HNPs) coated with DSPE-PEG vs. C14-TPA-PEG in luciferase-expressing HeLa cells. Lipofectamine 2000 (lipo2K) was used as a positive control.

### DETAILED DESCRIPTION

Along with the vaccine success and its enormous potential, one unique challenge associated with mRNA therapy (or RNA therapies in general) is dealing with the transient efficacy due to its relatively short half-life. Whereas LNPs or other nanoparticles have shown the ability to significantly improve mRNA translation efficiency, the duration of in vivo protein expression by these mRNA nanoparticles is generally limited to ˜2-7 days, such as for FVIII or FIX^{11,12} By prolonging the duration of protein expression, mRNA therapy can become transformative for treatment of hemophilia, other genetic disorders, and many other diseases as described herein.

The use of synthetic mRNA as an alternative to plasmid DNA has recently attracted significant attention. Nanotechnology has been widely applied to improve mRNA delivery by addressing its certain unfavorable features (e.g., large molecular weight, negative charge, and susceptibility to nuclease degradation). A variety of nanoparticle (NP) platforms have shown to improve RNA delivery, among which lipid NPs (LNPs) represent the most appealing and commonly used delivery vehicle. Notably, the DLin-MC3-DMA (also referred to as MC3)-based LNPs are already approved for clinical use of siRNA therapy (Onpattro) for a genetic disease¹³, and two LNP-based mRNA vaccines are recently approved for COVID-19¹⁴, along with many RNA nanotherapeutics currently in clinical trials⁶. Despite these successes, one unique challenge associated with mRNA therapy is dealing with the transient activity due to its relatively short half-lives, therefore generally requiring frequent repeated dosing to sustain therapeutic levels of protein. The protein expression mediated by current mRNA NPs (including LNPs) generally peaks at ˜6-24 hours and its duration is mainly limited to ˜2-7 days (depending on the NP, protein, dose, and cell/animal model; see Table 1 below).

TABLE 1
Summary of mRNA NPs-mediated protein expression in the literature

| mRNA- | | mRNA dose | | | | | | | |
|---|---|---|---|---|---|---|---|---|---|
| encoded | NP | (single | | | | | | Admin. | |
| protein | formulation | injection) | | Peak time | | Duration | | route | Ref. |
| OX40L | Lipid NPs | 5 | μg | 6 | h | 7 | d | Intratumor | ¹⁵ |
| hEPO | Lipid NPs | 0.01 mg/kg | | 6 h (monkey) | | 3 d (monkey) | | 60 min IV | ¹⁶ |
| | (MC3 and | (monkey) | | 2-4 h (rat) | | 2 d (rat) | | | |
| | lipid5) | 1 mg/kg (rat) | | | | | | | |
| Frataxin | Lipid NPs | 1 mg/kg | | 6 | h | <24 | h | IV | ¹⁷ |
| (FXN) and | (MC3) | (iFXN) | | | | | | | |
| luciferase | | 0.2 mg/kg | | 6 | h | 2-6 | d | ICV (intra- | |
| | | (luciferase) | | | | | | cerebroventricular) | |
| Luciferase | Lipid NPs | 5 | μg | 4.8 | h | 3 d (<0.1%) | | IV | ⁹ |
| | (MC3) | | | | | | | | |
| Luciferase | Lipid NPs | 0.25 | mg/kg | 6 | h | 2 | d | IV | ¹⁸ |
| ADAMTS13 | Lipid NPs | 1 | mg/kg | 24 | h | 5 | d | IV | ¹⁹ |
| Arg1 | Lipid NPs | 1.5 | mg/kg | 24 | h | 7 | d | IV | ²⁰ |
| Luciferase | Lipid NPs | 2 | mg/kg | 12 h (luc) | | 3 d (luc) | | IV | ²¹ |
| and Arg1 | | | | 2 h (Arg1) | | 2 d (Arg1) | | | |
| Factor IX | Lipid NPs | 4 | mg/kg | 24 | h | >4 | d | IV | ²² |
| Factor IX, | Lipid NPs | 1 | mg/kg | 6-12 | h | 3 | d | IV | ¹² |
| EPO | | | | | | | | | |
| hEPO | Lipid NPs | 0.3 | mg/kg | 6 | h | 7 | d | IV | ²³ |
| Factor VIII | TransIT | 3 | μg | 6-8 | h | 3 | d | IV | ²⁴ |
| | agent | | | | | | | | |
| Luciferase | Lipid NPs | 400 | ng | 24 | h | 6 | d | Subretinal | ²⁵ |
| | (MC3) | | | | | | | | |
| GFP, | Polymeric | 0.5 | mg/kg | 12 | h | 7 d (GFP) | | Subcutaneous | ²⁶ |
| Follistatin | NPs | | | | | 4 d (FS) | | (s.c.) | |
| Luciferase | Polymeric | 7.5 | μg | 8 h (IV) | | 2 | d | IV, s.c. | ²⁷ |
| | NPs | | | 4 h (s.c.) | | | | | |
| Luciferase | PEG- | 1 | μg | 4 | h | 3 | d | Hydrodynamic | ²⁸ |
| | peptide | | | | | | | | |
| | NPs | | | | | | | | |
| Luciferase | Lipid- | 1 | mg/kg | 12 | h | 4 | d | IV | ²⁹ |
| | polymer | | | | | | | | |
| | hybrid NPs | | | | | | | | |
| Luciferase | Lipid NPs | 0.1 | mg/kg | 4 | h | >2 | d | IV | ³⁰ |

To address this challenge of mRNA therapy, described herein is a delivery technology that can prolong the expression duration of model proteins, e.g., by at least 2, 3, 4, 5, 6, 7, 8, 9, or 10 folds. This may thus significantly reduce the dosing frequency, which will i) mitigate treatment burden; ii) improve patient compliance and quality of life, and iii) lower treatment costs.

As shown herein, by coating or including a distinct type of lipid-PEG or lipid-PEG-like molecules (Cn-X-PEG) on or in the mRNA NP surface (e.g., incorporating the lipid-PEG into the surface), the in vitro expression of model proteins (e.g., GFP) can be extended to more than 30 days. When using the Cn-X-PEG-coated LNPs for mRNA delivery in vivo, the expression of luciferase and human erythropoietin in normal C57BL/6 mice lasted ˜14 days and >21 days, respectively, after a single intravenous injection, much longer than that by traditional mRNA NPs (Table 1). In addition to extending the duration of mRNA activities in vitro and in vivo, the Cn-X-PEGs described herein can also dramatically prolong the NP circulation in the blood. The Cn-X-PEG-coated NPs could also be expanded for delivery of other nucleic acids (such as siRNA and antisense), biomacromolecules in general (such as enzymes and antibodies), peptides, small molecules, and imaging agents. These unique features of the Cn-X-PEGs as described herein can lead to development of new and effective nanotherapies for diverse biomedical applications. The studies disclosed herein suggest that the mRNA LNPs of the invention could be used for long-term durable protein replacement for genetic diseases such as hemophilia, ornithine transcarbamylase (OTC) deficiency, thrombotic thrombocytopenic purpura, methylmalonic academia, hereditary tyrosinemia type 1, Fabry disease, acute intermittent porphyria, alpha-1 antitrypsin deficiency, glycogen storage disease type 1, cystic fibrosis, and others. This technology can also be applied to other disease types such as cancer, pain, genetic blood disorders, genetic disorders that are characterized by protein deficiencies or malfunctions (such as thrombotic thrombocytopenic purpura, methylmalonic academia, hereditary tyrosinemia type 1, Fabry disease, acute intermittent porphyria, alpha-1 antitrypsin deficiency, glycogen storage disease type 1, cystic fibrosis, and others), infectious diseases (i.e., diseases associated with or caused be an infectious agent such as a virus, e.g., a viral infection such as COVID-19), neurodegenerative diseases, diabetes, inflammatory diseases, metabolic diseases, cardiovascular diseases, cardiometabolic diseases, eye diseases, ear diseases, and others

### Lipid-PEG Molecules and Compositions

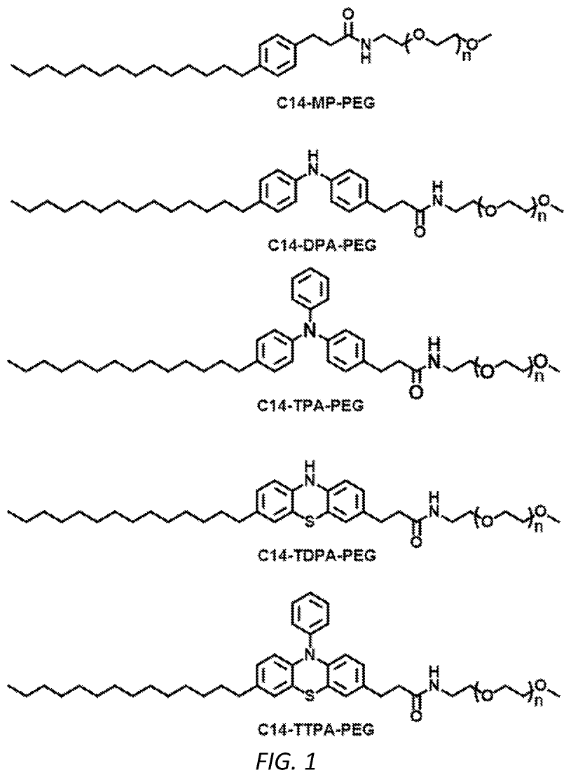

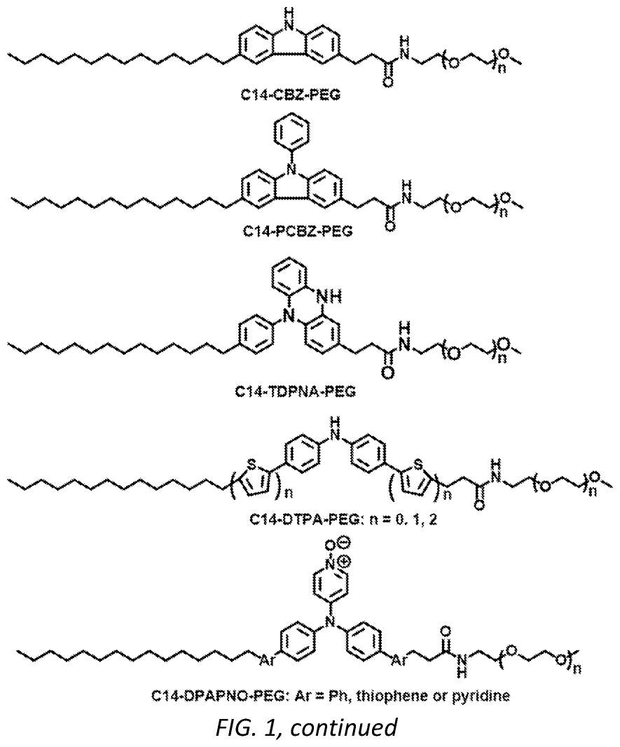

Described herein are compositions comprising a carbon chain, a central moiety comprising one or more heteroaryl groups, and a hydrophilic polymer (also referred to herein as Cn-X-PEG). The carbon length of the Cn, the middle functional group (X), and the molecular weight of the PEG can vary, and the PEG can be substituted with other hydrophilic polymers. For example, the length of the C chain can be from ˜2 to 40, e.g., 5 to 35. The molecular weight of the PEG can vary from ˜100 to 500,000, e.g., 500 to 5,000. In some embodiments, the PEG or other hydrophilic polymer is further linked to a ligand, e.g., galactose or N-acetylgalactosamine (GalNAc), or another targeting ligand as described herein. Some exemplary Cn-X-PEGs are shown in FIG. 1.

For example, described herein are compounds of Formula (I):

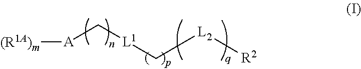

wherein:
A is selected from C₆₋₁₀ aryl and 5- to 10-membered heteroaryl, wherein the C₆₋₁₀ aryl or 5- to 10-membered heteroaryl is optionally substituted with one or more substituents independently selected from the group consisting of C₁₋₁₅ alkyl, C₂₋₁₅ alkenyl, C₂₋₁₅ alkynyl, C₃₋₁₀ cycloalkyl, C₆₋₁₀ aryl, 5- to 10-membered heteroaryl, 4- to 10-membered heterocycloalkyl, halo, —CN, —OR^{O}, —N(R^{N})₂, —(C═O)R^{N}, —(C═O)OR^{O}, —(C═O)N(R^{N})₂, and —NR^{N}(C═O)R⁸;
each R^{1A} is selected from C₁₋₁₀₀ alkyl, C₁₋₁₀₀ alkenyl, C₁₋₁₀₀ alkynyl, and C₁₋₁₀₀ haloalkyl, wherein the C₁₋₁₀₀ alkyl, C₁₋₁₀₀ alkenyl, and C₁₋₁₀₀ alkynyl forming R¹ is optionally substituted with one or more substituents independently selected from the group consisting of halo, —CN, —OR^{O}, —N(R^{N})₂, —(C═O)R^{N}, —(C═O)OR^{O}, —(C═O)N(R^{N})₂, —NR^{N}(C═O)—, and —O(C═O)R⁸;
L¹ is selected from bond, —N(R^{N})—, —O—, —(C═O)—, —(C═O)O—, —(C═O)N(R^{N})—, —NR^{N}(C═O)—, and —O(C═O)—;
L² is selected from:

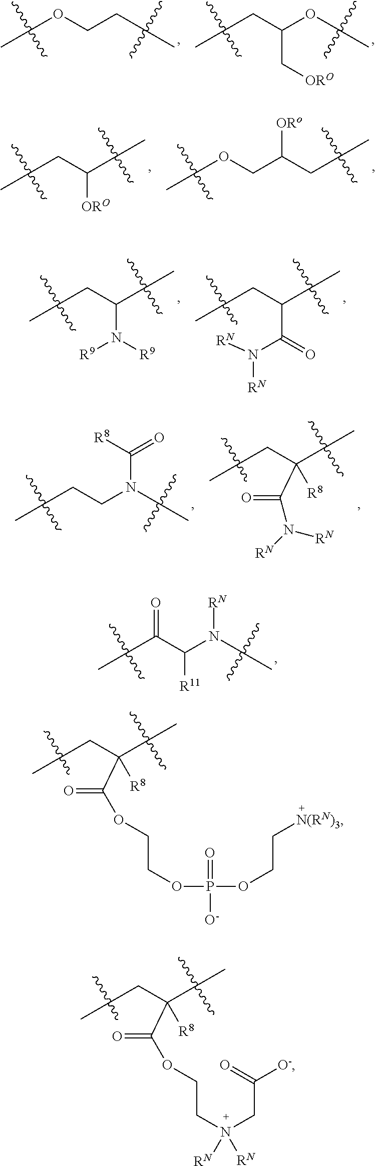

heparin, dextran, and chitosan;
R² is selected from H, C₁₋₁₅ alkyl, C₂₋₁₅ alkenyl, C₂₋₁₅ alkynyl, —OR^{O}, —(C═O)OR^{O}, —N(R^{N})₂, —N₃,

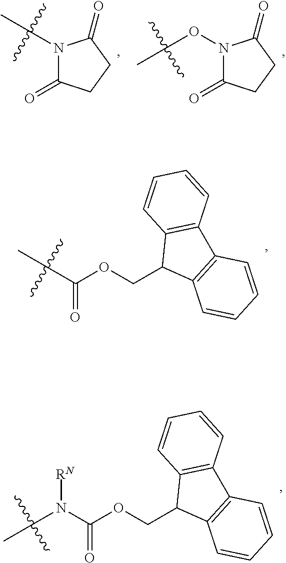

and targeting ligand;
each R⁸ is independently selected from H, C₁₋₁₅ alkyl, C₂₋₁₅ alkenyl, and C₂₋₁₅ alkynyl;
each R⁹ is independently selected from H, C₁₋₁₅ alkyl, C₂₋₁₅ alkenyl, and C₂₋₁₅ alkynyl;
or two R⁹, together with the N atom to which they are attached, come together to form 4- to 10-membered heterocycloalkyl optionally substituted with one or more oxo;
each R¹¹ is independently selected from C₁₋₁₅ alkyl, C₂₋₁₅ alkenyl, and C₂₋₁₅ alkynyl, optionally substituted with one or more R¹²;
each R¹² is independently selected from C₃₋₁₀ cycloalkyl, C₆₋₁₀ aryl, 5- to 10-membered heteroaryl, 4- to 10-membered heterocycloalkyl, halo, —CN, —OR^{O}, —N(R^{N})₂, —(C═O)R^{N}, —(C═O)OR^{O}, —(C═O)N(R^{N})₂, —NR^{N}(C═O)R⁸, —NR^{N}(C═NR^{N})R^{N}, —(C═O)R⁸, and —SR⁸, wherein the C₃₋₁₀ cycloalkyl, C₆₋₁₀ aryl, 5- to 10-membered heteroaryl, 4- to 10-membered heterocycloalkyl is optionally substituted with one or more C₁₋₁₅ alkyl, C₂₋₁₅ alkenyl, and C₂₋₁₅ alkynyl, halo, —CN, —OR^{O}, and —N(R^{N})₂;
each R^{N} is independently selected from H, C₁₋₁₅ alkyl, C₂₋₁₅ alkenyl, and C₂₋₁₅ alkynyl, wherein the C₁₋₁₅ alkyl, C₂₋₁₅ alkenyl, and C₂₋₁₅ alkynyl forming R^{N} is optionally substituted with one or more substituents independently selected from the group consisting of halo, —CN, and —OR^{O};
each R^{O} is independently selected from H, C₁₋₁₅ alkyl, C₂₋₁₅ alkenyl, and C₂₋₁₅ alkynyl, wherein the C₁₋₁₅ alkyl, C₂₋₁₅ alkenyl, and C₂₋₁₅ alkynyl forming R^{O} is optionally substituted with one or more substituents independently selected from the group consisting of halo and —CN;
m is an integer selected from 1, 2, 3, 4, and 5;
n and p are each an integer independently selected from 0, 1, 2, 3, and 4;
q is an integer selected from 1 to 2500;
provided that when A is phenyl, then each R^{1A} is selected from C₁₃₋₁₀₀ alkyl, C₁₋₁₀₀ alkenyl, C₁₋₁₀₀ alkynyl, and C₁₋₁₀₀ haloalkyl, wherein the C₁₃₋₁₀₀ alkyl, C₁₋₁₀₀ alkenyl, and C₁₋₁₀₀ alkynyl forming R¹ is optionally substituted with one or more substituents independently selected from the group consisting of halo, —CN, —OR^{O}, —N(R^{N})₂, —(C═O)R^{N}, —(C═O)OR^{O}, —(C═O)N(R^{N})₂, —NR^{N}(C═O)R⁸, and —O(C═O)R⁸.

In some embodiments, the compound is of Formula (IA):

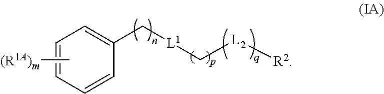

In some embodiments, the compound is of Formula (IA-1):

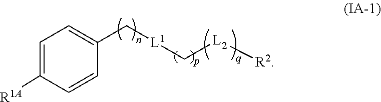

In some embodiments, the compound is of Formula (IA-2):

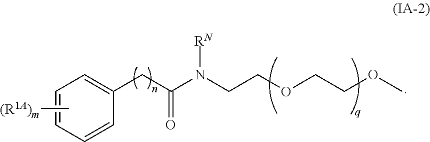

In some embodiments, the compound is of Formula (IA-3):

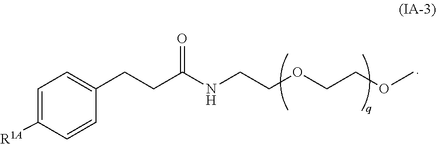

In some embodiments, the compound is of Formula (IA-4):

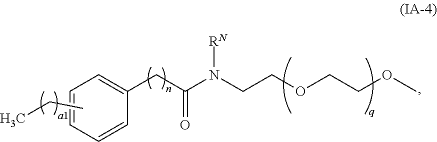

wherein a1 is an integer selected from 12 to 100.

In some embodiments, the compound is of Formula (IA-5):

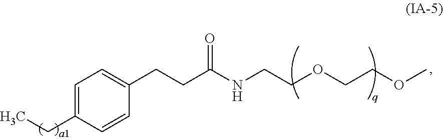

wherein a1 is an integer selected from 12 to 100.

In some embodiments, the compound is of Formula (I-1):

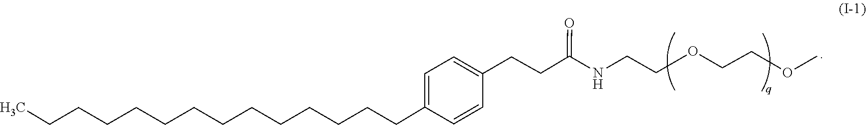

In some embodiments, the compound is a compound of Formula (I); A is C₆₋₁₀ aryl; each R^{1A} is C₁₃₋₁₀₀ alkyl; L¹ is —(C═O)N(R^{N})—; and L² is

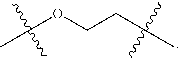 [SMILES: *CCO*]

In some embodiments, the compound is a compound of Formula (I) and A is C₆₋₁₀ aryl. In some embodiments, the compound is a compound of Formula (I) and A is phenyl. In some embodiments, the compound is a compound of Formula (I) and A is 5- to 10-membered heteroaryl. In some embodiments, the compound is a compound of Formula (I) and A is pyridinyl. In some embodiments, the compound is a compound of Formula (I) and A is thiophenyl. In some embodiments, the compound is a compound of Formula (I) and A is furanyl. In some embodiments, the compound is a compound of Formula (I) and A is pyrrolyl.

In some embodiments, the compound is a compound of Formula (I) and at least one R^{1A} is C₁₋₁₀₀ alkyl. In some embodiments, the compound is a compound of Formula (I) and at least one R^{1A} is C₁₋₄₀ alkyl. In some embodiments, the compound is a compound of Formula (I) and at least one R^{1A} is C₁₋₂₀ alkyl. In some embodiments, the compound is a compound of Formula (I) and at least one R^{1A} is C₁₄ alkyl. In some embodiments, the compound is a compound of Formula (I) and at least one R^{1A} is C₂₀₋₄₀ alkyl. In some embodiments, the compound is a compound of Formula (I) and at least one R^{1A} is C₁₋₁₀₀ alkenyl. In some embodiments, the compound is a compound of Formula (I) and at least one R^{1A} is C₁₋₁₀₀ alkynyl. In some embodiments, the compound is a compound of Formula (I) and at least one R^{1A} is C₁₋₁₀₀ haloalkyl.

In some embodiments, the compound is a compound of Formula (I), A is phenyl, and at least one R^{1A} is C₁₃₋₁₀₀ alkyl. In some embodiments, the compound is a compound of Formula (I), A is phenyl, and at least one R^{1A} is C₁₃₋₄₀ alkyl. In some embodiments, the compound is a compound of Formula (I) A is phenyl, and at least one R^{1A} is C₁₃₋₂₀ alkyl. In some embodiments, the compound is a compound of Formula (I) A is phenyl, and at least one R^{1A} is C₁₄ alkyl. In some embodiments, the compound is a compound of Formula (I) A is phenyl, and at least one R^{1A} is C₂₀₋₄₀ alkyl. In some embodiments, the compound is a compound of Formula (I), A is phenyl, and at least one R^{1A} is C₁₋₁₀₀ haloalkyl.

In some embodiments, the compound is a compound of Formula (I) and L¹ is bond. In some embodiments, the compound is a compound of Formula (I) and L¹ is —N(R^{N})—. In some embodiments, the compound is a compound of Formula (I) and L¹ is —O—. In some embodiments, the compound is a compound of Formula (I) and L¹ is —(C═O)—. In some embodiments, the compound is a compound of Formula (I) and L¹ is —(C═O)O—. In some embodiments, the compound is a compound of Formula (I) and L¹ is —(C═O)N(R^{N})—. In some embodiments, the compound is a compound of Formula (I) and L¹ is —(C═O)NH—. In some embodiments, the compound is a compound of Formula (I) and L¹ is —NR^{N}(C) In some embodiments, the compound is a compound of Formula (I) and L¹ is —O(C═O)—.

In some embodiments, the compound is a compound of Formula (I) and L² is

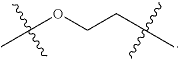 [SMILES: *CCO*]

In some embodiments, the compound is a compound of Formula (I) and L² is

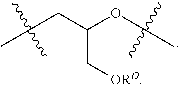

In some embodiments, the compound is a compound of Formula (I) and L² is

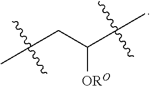

In some embodiments, the compound is a compound of Formula (I) and L² is

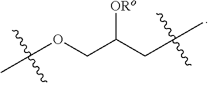

In some embodiments, the compound is a compound of Formula (I) and L² is

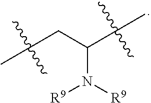

In some embodiments, the compound is a compound of Formula (I) and L² is

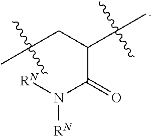

In some embodiments, the compound is a compound of Formula (I) and L² is

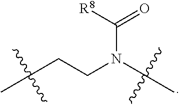

In some embodiments, the compound is a compound of Formula (I) and L² is

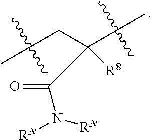

In some embodiments, the compound is a compound of Formula (I) and L² is

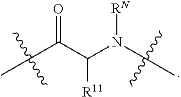

In some embodiments, the compound is a compound of Formula (I) and L² is

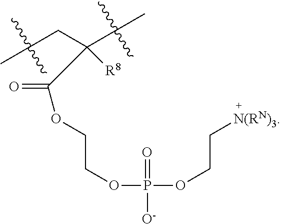

In some embodiments, the compound is a compound of Formula (I) and L² is

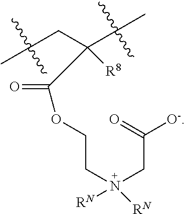

In some embodiments, the compound is a compound of Formula (I) and L In some embodiments, the compound is a compound of Formula (I) and L² is is heparin. In some embodiments, the compound is a compound of Formula (I) and L² is dextran. In some embodiments, the compound is a compound of Formula (I) and L² is chitosan.

In some embodiments, the compound is a compound of Formula (I) and L² is selected from

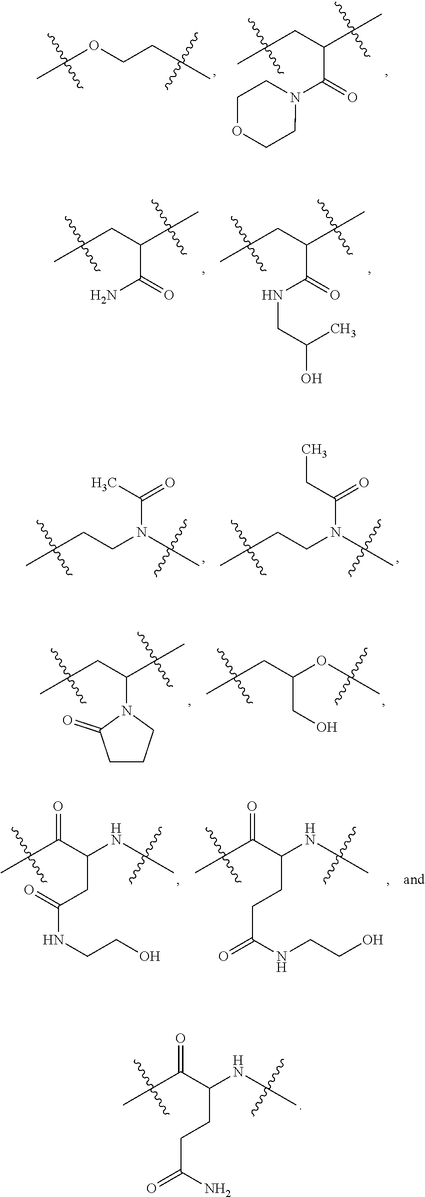

In some embodiments, the compound is a compound of Formula (I) and R² is H. In some embodiments, the compound is a compound of Formula (I) and R² is C₁₋₁₅ alkyl. In some embodiments, the compound is a compound of Formula (I) and R² is C₂₋₁₅ alkenyl. In some embodiments, the compound is a compound of Formula (I) and R² is C₂₋₁₅ alkynyl. In some embodiments, the compound is a compound of Formula (I) and R² is —OR^{O}. In some embodiments, the compound is a compound of Formula (I) and R² is —OCH₃. In some embodiments, the compound is a compound of Formula (I) and R² is —OC(CH₃)₃. In some embodiments, the compound is a compound of Formula (I) and R² is —(C═O)OR^{O}. In some embodiments, the compound is a compound of Formula (I) and R² is —N(R^{N})₂. In some embodiments, the compound is a compound of Formula (I) and R² is —N₃. In some embodiments, the compound is a compound of Formula (I) and R² is

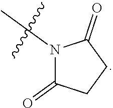 [SMILES: *N1C(=O)CCC1=O]

embodiments, the compound is a compound of Formula (I) and R² is

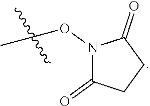 [SMILES: *ON1C(=O)CCC1=O]

In some embodiments, the compound is a compound of Formula (I) and R² is

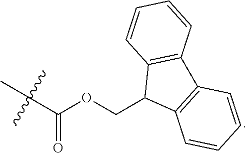 [SMILES: *C(=O)OCC1c2ccccc2-c2ccccc21]

In some embodiments, the compound is a compound of Formula (I) and R² is

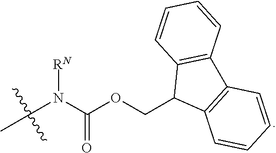

In some embodiments, the compound is a compound of Formula (I) and R² is a targeting ligand.

In some embodiments, the targeting ligand is selected from a protein, a monosaccharide, a polysaccharide, a peptide, an aptamer, a small molecule, and a nucleic acid-based ligand.

In some embodiments, the targeting ligand is a protein and the protein is selected from an antibody, a transferrin, an ankyrin repeat protein, and an affibody.

In some embodiments, the targeting ligand is an antibody, and the antibody is selected from an F(ab′)2 fragment, an F(ab′) fragment, and a single-chain variable fragment.

In some embodiments, the targeting ligand is a monosaccharide and the monosaccharide is selected from glucose, fructose, galactose, xylose, ribose, and N-acetylgalactosamine (GalNAc).

In some embodiments, the targeting ligand is selected from galactose and N-acetylgalactosamine (GalNAc).

In some embodiments, the targeting ligand is galactose.

In some embodiments, the targeting ligand is N-acetylgalactosamine (GalNAc).

In some embodiments, the targeting ligand is a polysaccharide and the polysaccharide is hyaluronic acid.

In some embodiments, the targeting ligand is a peptide and the peptide is selected from RGD, IL4RPep-1, viral envelope peptide, angiopep-2, and Asn-Gly-Arg peptide.

In some embodiments, the targeting ligand is an aptamer and the aptamer is selected from AS-1411, GB1-10, CGNKRTRGC (Lyp-1), F3 peptide, iRGD, KLWVLPKGGGC, KLWVLPK, and an aptide.

In some embodiments, the targeting ligand is a small molecule and the small molecule is selected from folate, folic acid, anisamide, phenylboronic acid, thiamine pyrophosphate (TPP), ((S)-2-(3-((S)-5-amino-1-carboxypentyl) ureido) pentanedioic acid (ACUPA), and 2-[3-(1, 3-dicarboxy propyl)-ureido] pentanedioic acid (DUPA). In some embodiments, the targeting ligand is a nucleic acid-based ligand and the nucleic acid-based ligand is selected from A10 aptamer, and A9 CGA aptamer.

In some embodiments, the compound is a compound of Formula (I) and at least one R⁸ is H. In some embodiments, the compound is a compound of Formula (I) and at least one R⁸ is C₁₋₁₅ alkyl. In some embodiments, the compound is a compound of Formula (I) and at least one R⁸ is C₂₋₁₅ alkenyl. In some embodiments, the compound is a compound of Formula (I) and at least one R⁸ is C₂₋₁₅ alkynyl.

In some embodiments, the compound is a compound of Formula (I) and at least one R⁹ is H. In some embodiments, the compound is a compound of Formula (I) and at least one R⁹ is C₁₋₁₅ alkyl. In some embodiments, the compound is a compound of Formula (I) and at least one R⁹ is C₂₋₁₅ alkenyl. In some embodiments, the compound is a compound of Formula (I) and at least one R⁹ is C₂₋₁₅ alkynyl. In some embodiments, the compound is a compound of Formula (I) and two R⁹, together with the N atom to which they are attached, come together to form 4- to 10-membered heterocycloalkyl optionally substituted with one or more oxo. In some embodiments, the compound is a compound of Formula (I) and two R⁹, together with the N atom to which they are attached, come together to form 5-membered heterocycloalkyl substituted with one or more oxo.

In some embodiments, the compound is a compound of Formula (I) and at least one R¹¹ is C₁₋₁₅ alkyl. In some embodiments, the compound is a compound of Formula (I) and at least one R¹¹ is C₁₋₁₅ alkyl substituted with one or more R¹². In some embodiments, the compound is a compound of Formula (I) and at least one R¹¹ is C₂₋₁₅ alkenyl. In some embodiments, the compound is a compound of Formula (I) and at least one R¹¹ is C₂₋₁₅ alkenyl substituted with one or more R¹². In some embodiments, the compound is a compound of Formula (I) and at least one R¹¹ is C₂₋₁₅ alkynyl. In some embodiments, the compound is a compound of Formula (I) and at least one R¹¹ is C₂₋₁₅ alkynyl substituted with one or more R¹².

In some embodiments, the compound is a compound of Formula (I) and at least one R¹² is C₃₋₁₀ cycloalkyl. In some embodiments, the compound is a compound of Formula (I) and at least one R¹² is C₃₋₁₀ cycloalkyl optionally substituted with one or more C₁₋₁₅ alkyl, C₂₋₁₅ alkenyl, and C₂₋₁₅ alkynyl, halo, —CN, —OR^{O}, and —N(R^{N})₂. In some embodiments, the compound is a compound of Formula (I) and at least one R¹² is C₆₋₁₀ aryl. In some embodiments, the compound is a compound of Formula (I) and at least one R¹² is C₆₋₁₀ aryl optionally substituted with one or more C₁₋₁₅ alkyl, C₂₋₁₅ alkenyl, and C₂₋₁₅ alkynyl, halo, —CN, —OR^{O}, and —N(R^{N})₂. In some embodiments, the compound is a compound of Formula (I) and at least one R¹² is 5- to 10-membered heteroaryl. In some embodiments, the compound is a compound of Formula (I) and at least one R¹² is 5- to 10-membered heteroaryl optionally substituted with one or more C₁₋₁₅ alkyl, C₂₋₁₅ alkenyl, and C₂₋₁₅ alkynyl, halo, —CN, —OR^{O}, and —N(R^{N})₂. In some embodiments, the compound is a compound of Formula (I) and at least one R¹² is 4- to 10-membered heterocycloalkyl. In some embodiments, the compound is a compound of Formula (I) and at least one R¹² is 4- to 10-membered heterocycloalkyl optionally substituted with one or more C₁₋₁₅ alkyl, C₂₋₁₅ alkenyl, and C₂₋₁₅ alkynyl, halo, —CN, —OR^{O}, and —N(R^{N})₂. In some embodiments, the compound is a compound of Formula (I) and at least one R² is halo. In some embodiments, the compound is a compound of Formula (I) and at least one R¹² is —CN. In some embodiments, the compound is a compound of Formula (I) and at least one R¹² is —OR^{O}. In some embodiments, the compound is a compound of Formula (I) and at least one R¹² is —N(R^{N})₂. In some embodiments, the compound is a compound of Formula (I) and at least one R¹² is —(C═O)R^{N}. In some embodiments, the compound is a compound of Formula (I) and at least one R¹² is —(C═O)OR^{O}. In some embodiments, the compound is a compound of Formula (I) and at least one R¹² is —(C═O)N(R^{N})₂. In some embodiments, the compound is a compound of Formula (I) and at least one R¹² is —NR^{N}(C═O)R⁸. In some embodiments, the compound is a compound of Formula (I) and at least one R¹² is —NR^{N}(C═NR^{N})R^{N} In some embodiments, the compound is a compound of Formula (I) and at least one R¹² is —O(C═O)R⁸. In some embodiments, the compound is a compound of Formula (I) and at least one R¹² is —SR⁸.

In some embodiments, the compound is a compound of Formula (I) and at least one R^{N} is H. In some embodiments, the compound is a compound of Formula (I) and at least one R^{N} is C₁₋₁₅ alkyl. In some embodiments, the compound is a compound of Formula (I) and at least one R^{N} is C₁₋₁₅ alkyl optionally substituted with one or more substituents independently selected from the group consisting of halo, —CN, and —OR^{O}. In some embodiments, the compound is a compound of Formula (I) and at least one R^{N} is C₂₋₁₅ alkenyl. In some embodiments, the compound is a compound of Formula (I) and at least one R^{N} is C₂₋₁₅ alkenyl optionally substituted with one or more substituents independently selected from the group consisting of halo, —CN, and —OR^{O}. In some embodiments, the compound is a compound of Formula (I) and at least one R^{N} is C₂₋₁₅ alkynyl. In some embodiments, the compound is a compound of Formula (I) and at least one R^{N} is C₂₋₁₅ alkynyl optionally substituted with one or more substituents independently selected from the group consisting of halo, —CN, and —OR^{O}.

In some embodiments, the compound is a compound of Formula (I) and at least one R^{O} is C₁₋₁₅ alkyl optionally substituted with one or more substituents independently selected from the group consisting of halo and —CN. In some embodiments, the compound is a compound of Formula (I) and at least one R^{O} is C₂₋₁₅ alkenyl. In some embodiments, the compound is a compound of Formula (I) and at least one R^{O} is C₂₋₁₅ alkenyl optionally substituted with one or more substituents independently selected from the group consisting of halo and —CN. In some embodiments, the compound is a compound of Formula (I) and at least one R^{O} is C₂₋₁₅ alkynyl. In some embodiments, the compound is a compound of Formula (I) and at least one R^{O} is C₂₋₁₅ alkynyl optionally substituted with one or more substituents independently selected from the group consisting of halo and —CN.

In some embodiments, the compound is a compound of Formula (I) and m is 1. In some embodiments, the compound is a compound of Formula (I) and m is 2. In some embodiments, the compound is a compound of Formula (I) and m is 3. In some embodiments, the compound is a compound of Formula (I) and m is 4. In some embodiments, the compound is a compound of Formula (I) and m is 1. In some embodiments, the compound is a compound of Formula (I) and m is 5.

In some embodiments, the compound is a compound of Formula (I) and n is 0. In some embodiments, the compound is a compound of Formula (I) and n is 1. In some embodiments, the compound is a compound of Formula (I) and n is 2. In some embodiments, the compound is a compound of Formula (I) and n is 3. In some embodiments, the compound is a compound of Formula (I) and n is 4.

In some embodiments, the compound is a compound of Formula (I) and p is 0. In some embodiments, the compound is a compound of Formula (I) and p is 1. In some embodiments, the compound is a compound of Formula (I) and p is 2. In some embodiments, the compound is a compound of Formula (I) and p is 3. In some embodiments, the compound is a compound of Formula (I) and p is 4.

In some embodiments, the compound is a compound of Formula (I) and q is an integer selected from 1 to 2500. In some embodiments, the compound is a compound of Formula (I) and q is an integer selected from 1 to 2000. In some embodiments, the compound is a compound of Formula (I) and q is an integer selected from 1 to 1500. In some embodiments, the compound is a compound of Formula (I) and q is an integer selected from 1 to 1000. In some embodiments, the compound is a compound of Formula (I) and q is an integer selected from 1 to 500. In some embodiments, the compound is a compound of Formula (I) and q is an integer selected from 500 to 2500. In some embodiments, the compound is a compound of Formula (I) and q is an integer selected from 1000 to 2500. In some embodiments, the compound is a compound of Formula (I) and q is an integer selected from 1500 to 2500. In some embodiments, the compound is a compound of Formula (I) and q is an integer selected from 2000 to 2500. In some embodiments, the compound is a compound of Formula (I) and q is an integer selected from 500 to 2000. In some embodiments, the compound is a compound of Formula (I) and q is an integer selected from 1000 to 1500.

Provided herein is a compound of Formula (II):

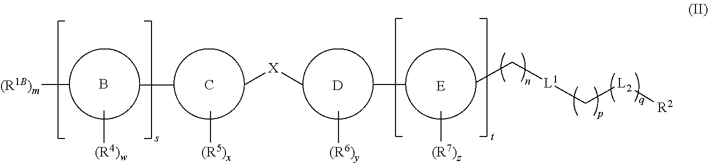

wherein:
X is selected from C(R³)₂, N³, O, S, and

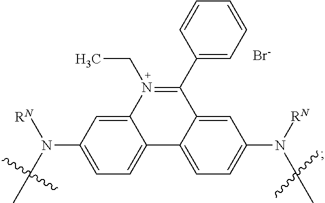

each R^{1B} is selected from C₁₋₁₀₀ alkyl, C₂₋₁₀₀ alkenyl, C₂₋₁₀₀ alkynyl, and C₁₋₁₀₀ haloalkyl, wherein the C₁₋₁₀₀ alkyl, C₁₋₁₀₀ alkenyl, and C₂₋₁₀₀ alkynyl forming R¹ is optionally substituted with one or more substituents independently selected from the group consisting of halo, —CN, —OR^{O}, —N(R^{N})₂, —(C═O)R^{N}, —(C═O)OR^{O}, —(C═O)N(R^{N})₂, —NR^{N}(C═O)R⁸, and —O(C═O)R⁸;
L¹ is selected from bond, —N(R^{N})—, —O—, —(C═O)—, —(C═O)O—, —(C═O)N(R^{N})—, —NR^{N}(C═O)—, and —O(C═O)—;
L² is selected from:

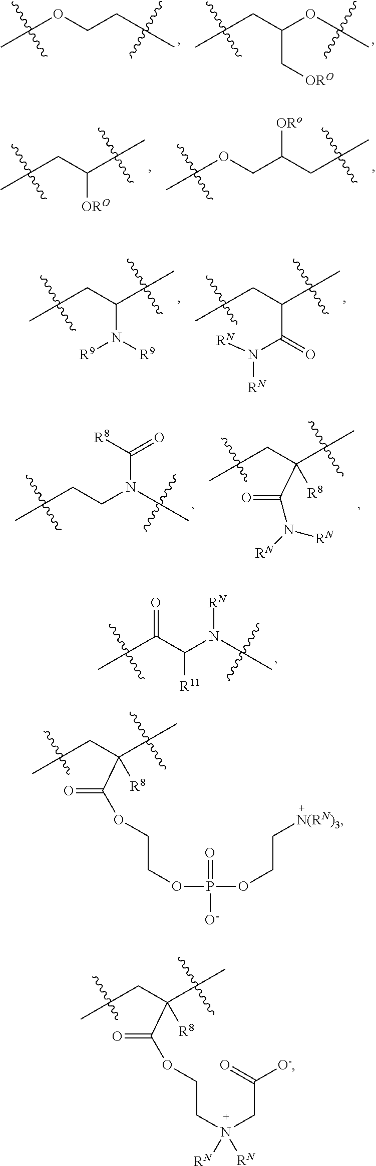

heparin, dextran, and chitosan;
R² is selected from H, C₁₋₁₅ alkyl, C₂₋₁₅ alkenyl, C₂₋₁₅ alkynyl, —OR^{O}, —(C═O)OR^{O}, —N(R^{N})₂, —N₃,

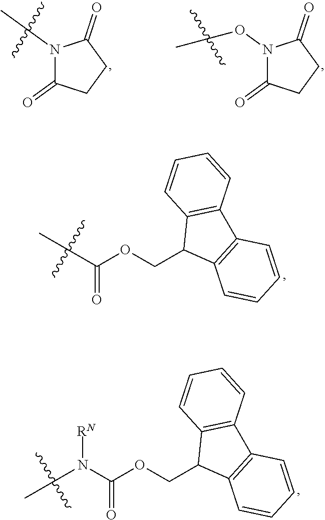

and targeting ligand;
each R³ is independently selected from H, C₁₋₁₅ alkyl, C₂₋₁₅ alkenyl, and C₂₋₁₅ alkynyl, C₃₋₁₀ cycloalkyl, C₆₋₁₀ aryl, 5- to 10-membered heteroaryl, and 4- to 10-membered heterocycloalkyl optionally substituted with one or more R¹⁰;
each Ring B, Ring C, Ring D, and Ring E is independently selected from C₆₋₁₀ aryl or 5- to 10-membered heteroaryl;
each R⁴, R⁵, R⁶, and R⁷ is independently selected from C₁₋₁₅ alkyl, C₂₋₁₅ alkenyl, C₂₋₁₅ alkynyl, C₃₋₁₀ cycloalkyl, C₆₋₁₀ aryl, 5- to 10-membered heteroaryl, 4- to 10-membered heterocycloalkyl, halo, —CN, —OR^{O}, —N(R^{N})₂, —(C═O)R^{N}, —(C═O)OR^{O}, —(C═O)N(R^{N})₂, —NR^{N}(C═O)R⁸, and —O(C═O)R⁸;
or an R⁵ and an R⁶, together with the atoms to which they are attached, come together to form C₆₋₁₀ aryl or 5- to 10-membered heteroaryl, wherein the C₆₋₁₀ aryl or 5- to 10-membered heteroaryl is optionally substituted with one or more R⁸;
each R⁸ is independently selected from H, C₁₋₁₅ alkyl, C₂₋₁₅ alkenyl, and C₂₋₁₅ alkynyl;
each R⁹ is independently selected from H, C₁₋₁₅ alkyl, C₂₋₁₅ alkenyl, and C₂₋₁₅ alkynyl;
or two R⁹, together with the N atom to which they are attached, come together to form 4- to 10-membered heterocycloalkyl optionally substituted with one or more oxo;
each R¹⁰ is independently selected from the group consisting of C₁₋₁₀₀ alkyl, C₂₋₁₀₀ alkenyl, C₂₋₁₀₀ alkynyl, C₁₋₁₀₀ haloalkyl, halo, —CN, —OR^{O}, oxo, —N(R^{N})₂, —(C═O)R^{N}, —(C═O)OR^{O}, —(C═O)N(R^{N})₂, —NR^{N}(C═O)R⁸, and —O(C═O)R⁸;
or an R⁵ and an R¹⁰, together with the atoms to which they are attached, come together to form C₆₋₁₀ aryl or 5- to 10-membered heteroaryl, wherein the C₆₋₁₀ aryl or 5- to 10-membered heteroaryl is optionally substituted with one or more R⁸;
or an R⁶ and an R¹⁰, together with the atoms to which they are attached, come together to form C₆₋₁₀ aryl or 5- to 10-membered heteroaryl, wherein the C₆₋₁₀ aryl or 5- to 10-membered heteroaryl is optionally substituted with one or more R⁸;
each R¹¹ is independently selected from C₁₋₁₅ alkyl, C₂₋₁₅ alkenyl, and C₂₋₁₅ alkynyl, optionally substituted with one or more R¹²;
each R¹² is independently selected from C₃₋₁₀ cycloalkyl, C₆₋₁₀ aryl, 5- to 10-membered heteroaryl, 4- to 10-membered heterocycloalkyl, halo, —CN, —OR^{O}, —N(R^{N})₂, —(C═O)R^{N}, —(C═O)R^{O}, —(C═O)N(R^{N})₂, —NR^{N}(C═O)R⁸, —NR^{N}(C═NR^{N})R^{N}, —(C═O)R⁸, and —SR, wherein the C₃₋₁₀ cycloalkyl, C₆₋₁₀ aryl, 5- to 10-membered heteroaryl, 4- to 10-membered heterocycloalkyl is optionally substituted with one or more C₁₋₁₅ alkyl, C₂₋₁₅ alkenyl, and C₂₋₁₅ alkynyl, halo, —CN, —OR^{O}, and —N(R^{N})₂;
each R^{N} is independently selected from H, C₁₋₁₅ alkyl, C₂₋₁₅ alkenyl, and C₂₋₁₅ alkynyl, wherein the C₁₋₁₅ alkyl, C₂₋₁₅ alkenyl, and C₂₋₁₅ alkynyl forming R^{N} is optionally substituted with one or more substituents independently selected from the group consisting of halo, —CN, and —OR^{O};
each R^{O} is independently selected from H, C₁₋₁₅ alkyl, C₂₋₁₅ alkenyl, and C₂₋₁₅ alkynyl, wherein the C₁₋₁₅ alkyl, C₂₋₁₅ alkenyl, and C₂₋₁₅ alkynyl forming R^{O} is optionally substituted with one or more substituents independently selected from the group consisting of halo and —CN;
m is an integer selected from 1, 2, 3, 4, and 5;
n and p are each an integer independently selected from 0, 1, 2, 3, and 4;
q is an integer selected from 1 to 2500;
s and t are each an integer independently selected from 0, 1, 2, 3, 4, and 5; and
w, x, y, and z are each an integer independently selected from 0, 1, 2, 3, and 4.

In some embodiments, the compound is of Formula (IIA):

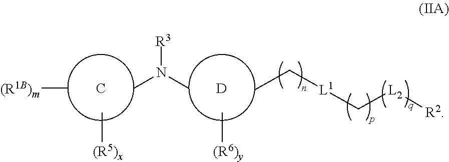

In some embodiments, the compound is of Formula (IIB):

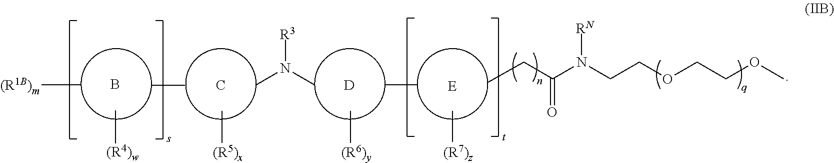

In some embodiments, the compound is of Formula (IIC):

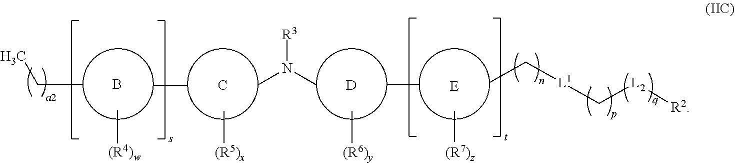

In some embodiments, the compound is of Formula (IID):

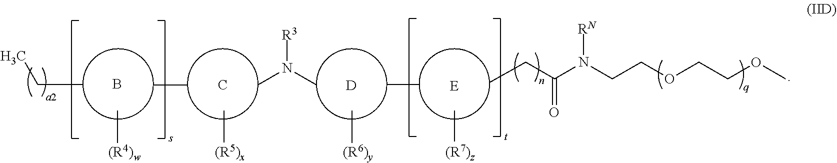

In some embodiments, the compound is selected from:

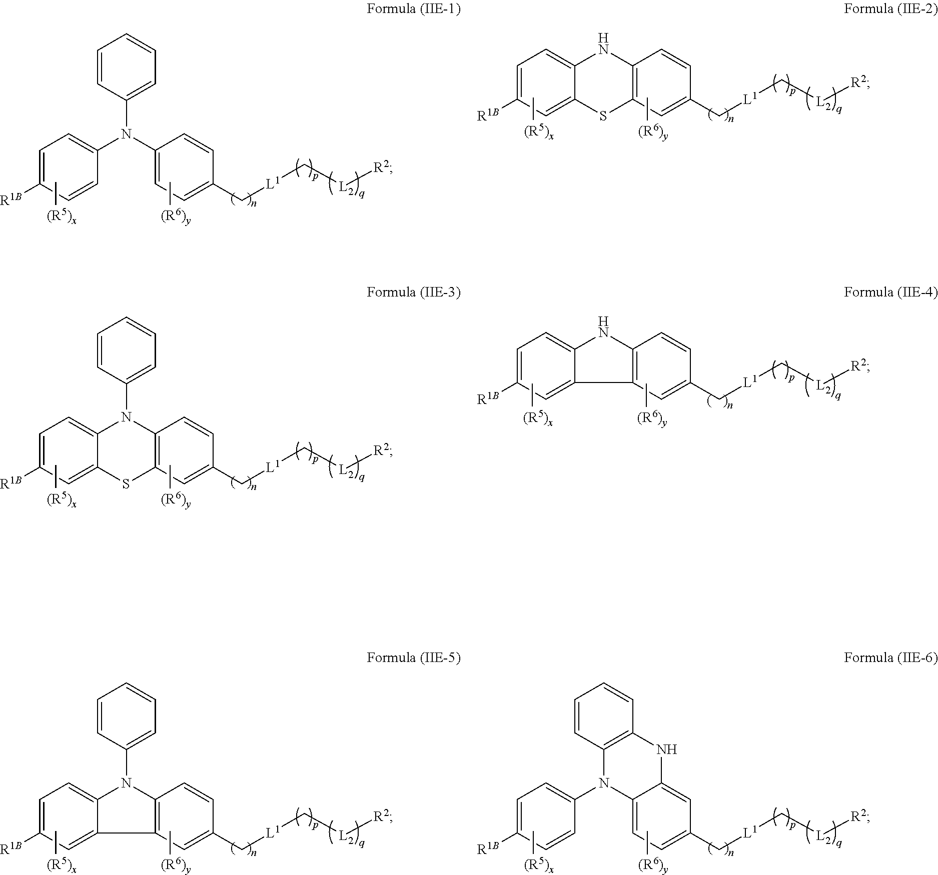

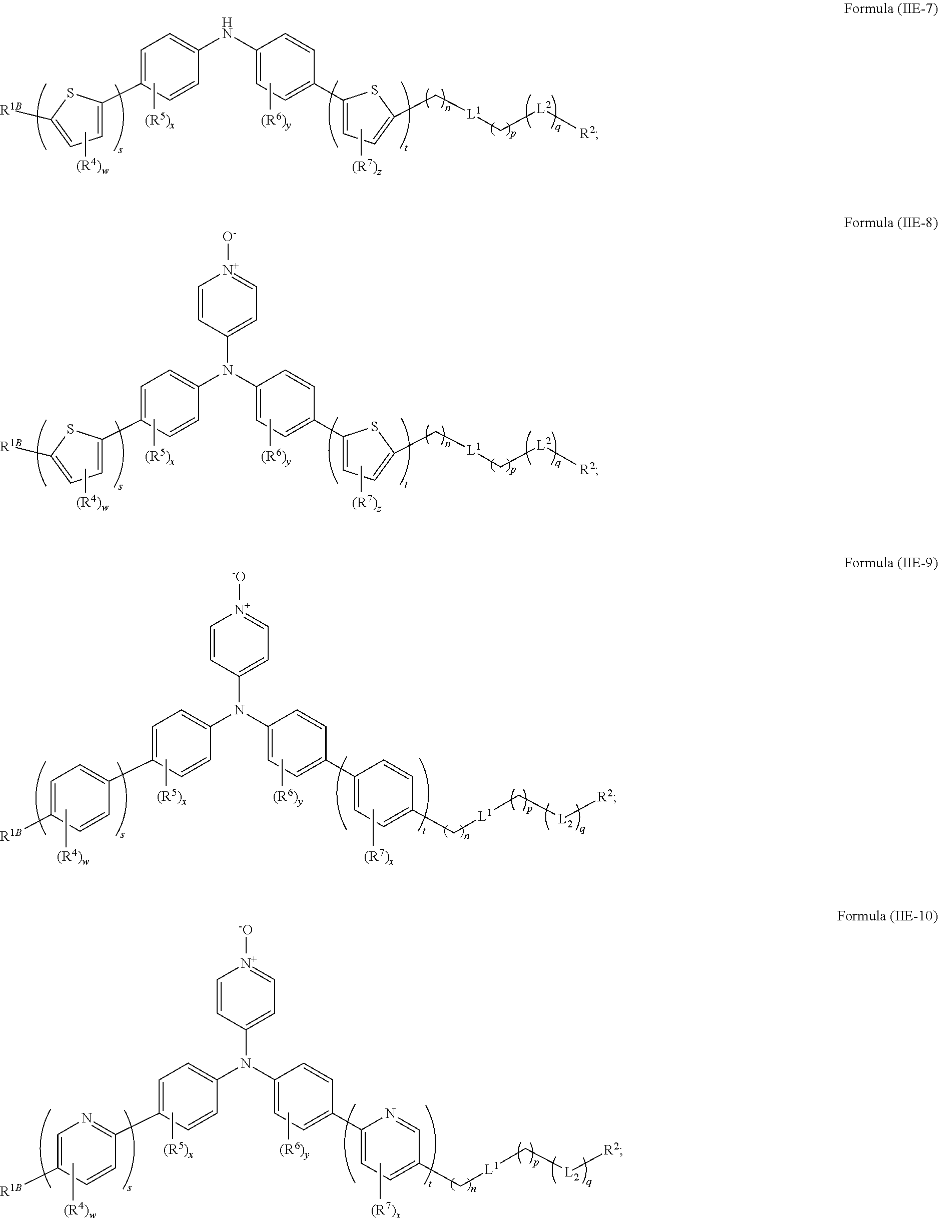

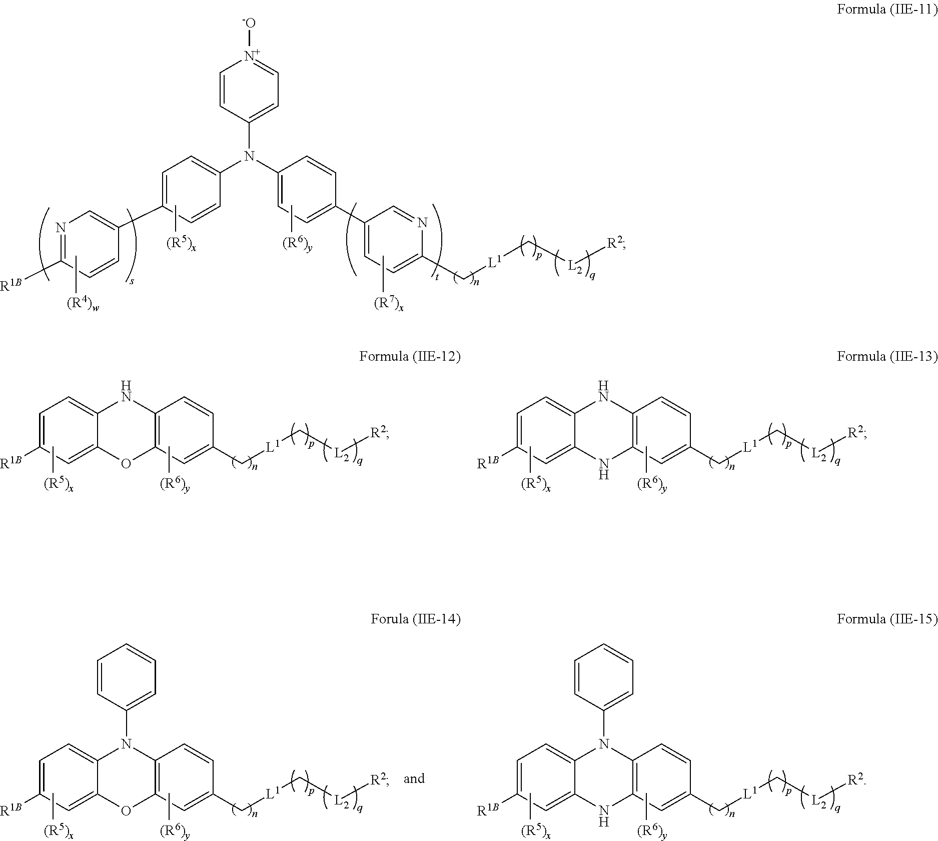

In some embodiments, the compound is selected from:

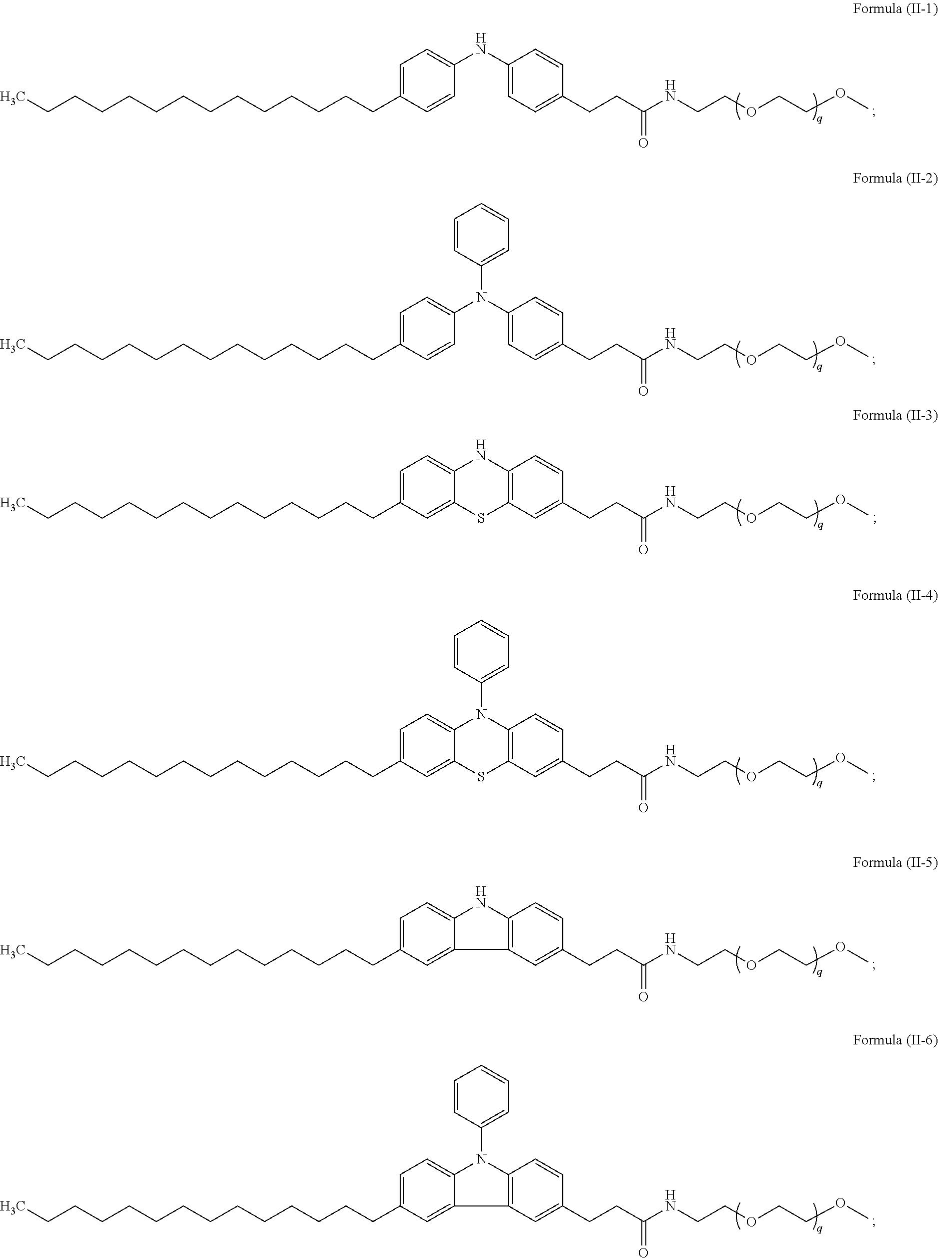

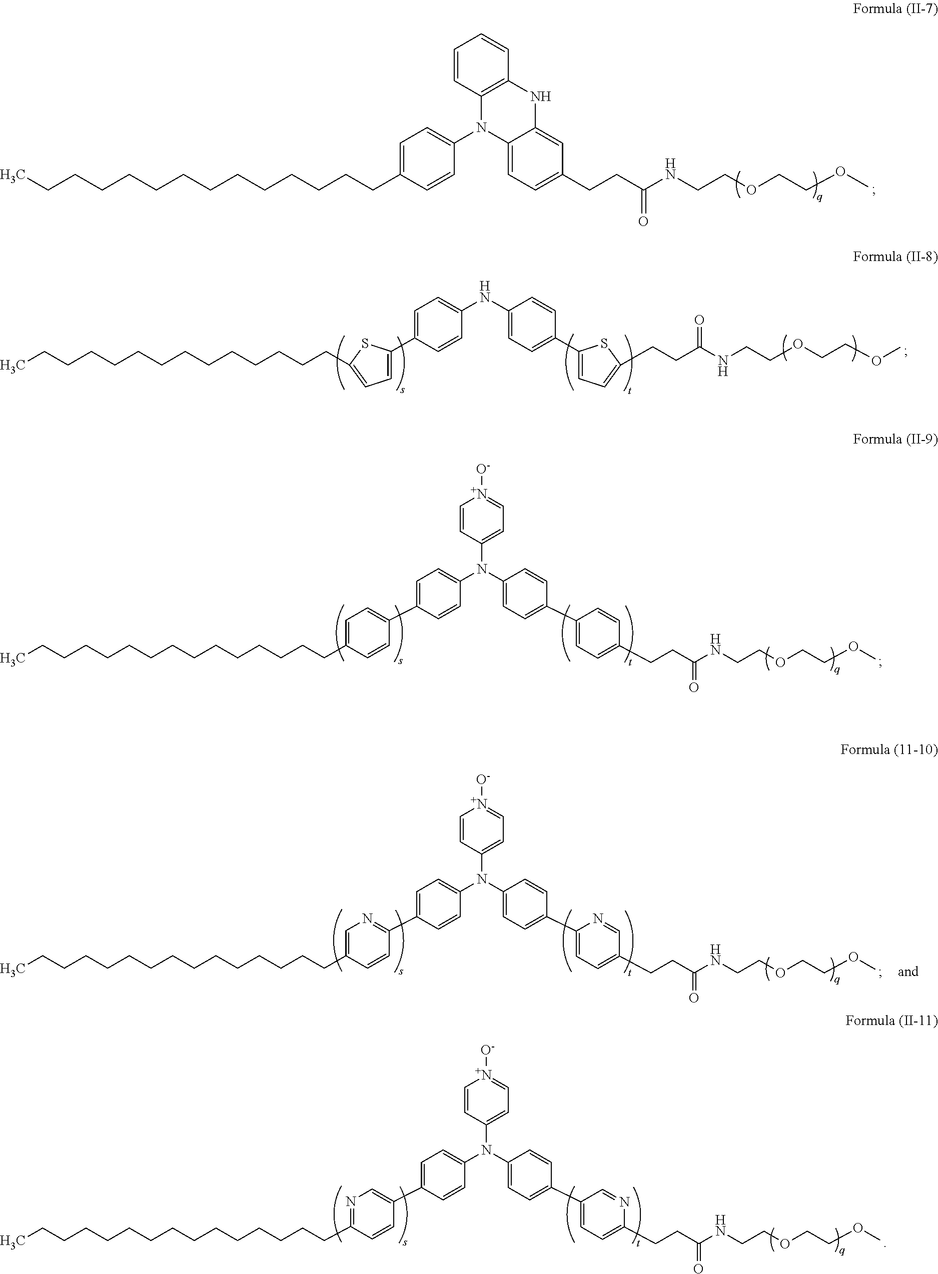

In some embodiments, the compound is a compound of Formula (II); each R^{1B}C₁₋₁₀₀ alkyl; L¹ is —(C═O)N(R^{N})—; L² is

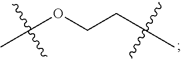 [SMILES: *CCO*]

R² is C₁₋₁₅ alkyl; R³ is selected from H and C₆₋₁₀ aryl; each R^{N} is H; and each R^{O} is C₁₋₁₅ alkyl.

In some embodiments, the compound is a compound of Formula (II) and X is C(R³)₂. In some embodiments, the compound is a compound of Formula (II) and X is NR³. In some embodiments, the compound is a compound of Formula (II) and X is O. In some embodiments, the compound is a compound of Formula (II) and X is S. In some embodiments, the compound is a compound of Formula (II) and X is

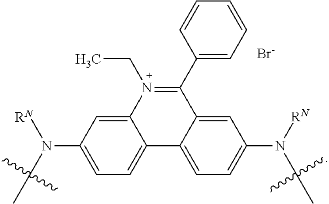

In some embodiments, the compound is a compound of Formula (II) and at least one R^{1B} is C₁₋₁₀₀ alkyl. In some embodiments, the compound is a compound of Formula (II) and at least one R^{1B} is C₁₋₄₀ alkyl. In some embodiments, the compound is a compound of Formula (II) and at least one R^{1B} is C₂₋₄₀alkyl. In some embodiments, the compound is a compound of Formula (II) and at least one R^{1B} is C₁₃₋₂₀ alkyl. In some embodiments, the compound is a compound of Formula (II) and at least one R^{1B} is C₁₄ alkyl. In some embodiments, the compound is a compound of Formula (II) and at least one R^{1B} is C₂₀₋₄₀ alkyl. In some embodiments, the compound is a compound of Formula (II) and at least one R^{1B} is C₁₋₁₀₀ alkenyl. In some embodiments, the compound is a compound of Formula (II) and at least one R^{1B} is C₁₋₁₀₀ alkynyl. In some embodiments, the compound is a compound of Formula (II) and at least one R^{1B} is C₁₋₁₀₀ haloalkyl.

In some embodiments, the compound is a compound of Formula (II) and L¹ is bond. In some embodiments, the compound is a compound of Formula (II) and L¹ is —N(R^{N})—. In some embodiments, the compound is a compound of Formula (II) and L¹ is —O—. In some embodiments, the compound is a compound of Formula (II) and L¹ is —(C═O)—. In some embodiments, the compound is a compound of Formula (II) and L¹ is —(C═O)O—. In some embodiments, the compound is a compound of Formula (II) and L¹ is —(C═O)N(R^{N})—. In some embodiments, the compound is a compound of Formula (II) and L¹ is —(C═O)NH—. In some embodiments, the compound is a compound of Formula (II) and L¹ is —NR^{N}(C═O)—. In some embodiments, the compound is a compound of Formula (II) and L¹ is —O(C═O)—.

In some embodiments, the compound is a compound of Formula (II) and L² is

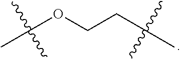 [SMILES: *CCO*]

In some embodiments, the compound is a compound of Formula (II) and L² is

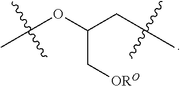

In some embodiments, the compound is a compound of Formula (II) and L² is

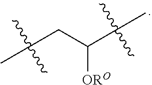

In some embodiments, the compound is a compound of Formula (II) and L² is

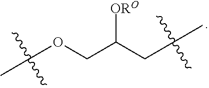

In some embodiments, the compound is a compound of Formula (II) and L² is

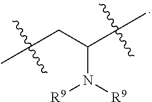

In some embodiments, the compound is a compound of Formula (II) and L² is

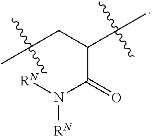

In some embodiments, the compound is a compound of Formula (II) and L² is

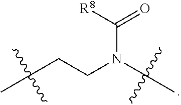

In some embodiments, the compound is a compound of Formula (II) and L² is

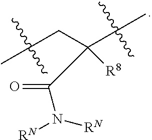

In some embodiments, the compound is a compound of Formula (II) and L² is

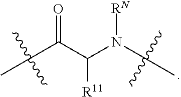

In some embodiments, the compound is a compound of Formula (II) and L² is

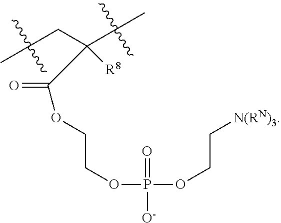

In some embodiments, the compound is a compound of Formula (II) and L² is

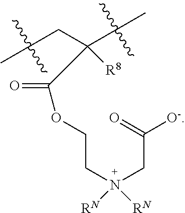

In some embodiments, the compound is a compound of Formula (I) and L In some embodiments, the compound is a compound of Formula (I) and L² is is heparin. In some embodiments, the compound is a compound of Formula (I) and L² is dextran. In some embodiments, the compound is a compound of Formula (I) and L² is chitosan.

In some embodiments, the compound is a compound of Formula (I) and L² is selected from

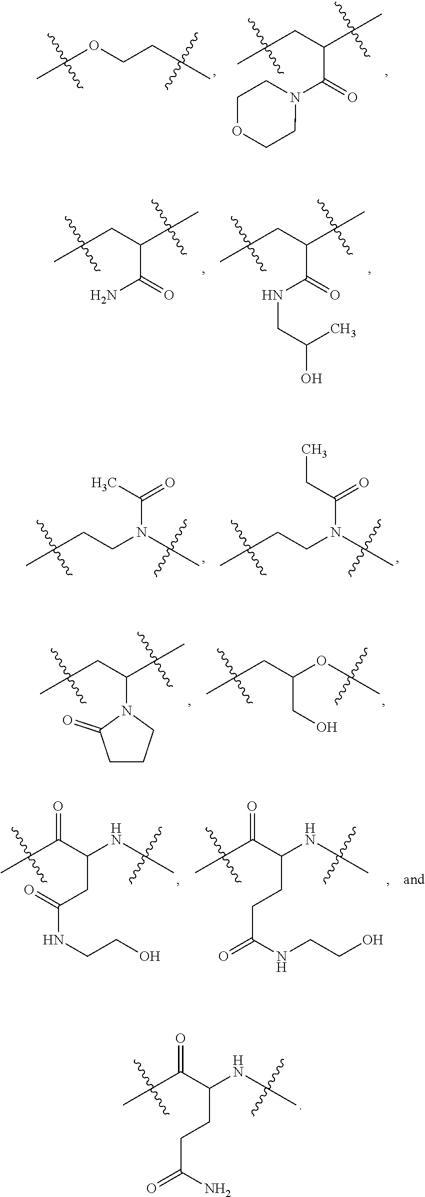

In some embodiments, the compound is a compound of Formula (II) and R² is H. In some embodiments, the compound is a compound of Formula (II) and R² is C₁₋₁₅ alkyl. In some embodiments, the compound is a compound of Formula (II) and R² is C₂₋₁₅ alkenyl. In some embodiments, the compound is a compound of Formula (II) and R² is C₂₋₁₅ alkynyl. In some embodiments, the compound is a compound of Formula (II) and R² is —OR^{O}. In some embodiments, the compound is a compound of Formula (II) and R² is —OCH₃. In some embodiments, the compound is a compound of Formula (II) and R² is —OC(CH₃)₃. In some embodiments, the compound is a compound of Formula (II) and R² is —(C═O)OR^{O}. In some embodiments, the compound is a compound of Formula (II) and R² is —N(R^{N})₂. In some embodiments, the compound is a compound of Formula (II) and R² is —N₃. In some embodiments, the compound is a compound of Formula (II) and R² is

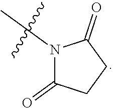 [SMILES: *N1C(=O)CCC1=O]

In some embodiments, the compound is a compound of Formula (II) and R² is

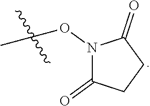 [SMILES: *ON1C(=O)CCC1=O]

In some embodiments, the compound is a compound of Formula (II) and R² is

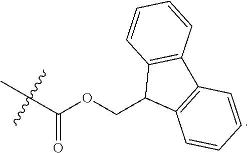 [SMILES: *C(=O)OCC1c2ccccc2-c2ccccc21]

In some embodiments, the compound is a compound of Formula (II) and R² is

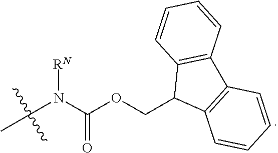

In some embodiments, the compound is a compound of Formula (II) and R² is a targeting ligand.

In some embodiments, the targeting ligand is selected from a protein, a monosaccharide, a polysaccharide, a peptide, an aptamer, a small molecule, and a nucleic acid-based ligand.

In some embodiments, the targeting ligand is a protein and the protein is selected from an antibody, a transferrin, an ankyrin repeat protein, and an affibody.

In some embodiments, the targeting ligand is an antibody, and the antibody is selected from an F(ab′)2 fragment, an F(ab′) fragment, and a single-chain variable fragment.

In some embodiments, the targeting ligand is a monosaccharide and the monosaccharide is selected from glucose, fructose, galactose, xylose, ribose, and N-acetylgalactosamine (GalNAc).

In some embodiments, the targeting ligand is selected from galactose and N-acetylgalactosamine (GalNAc).

In some embodiments, the targeting ligand is galactose.

In some embodiments, the targeting ligand is N-acetylgalactosamine (GalNAc).

In some embodiments, the targeting ligand is a polysaccharide and the polysaccharide is hyaluronic acid.

In some embodiments, the targeting ligand is a peptide and the peptide is selected from RGD, IL4RPep-1, viral envelope peptide, angiopep-2, and Asn-Gly-Arg peptide.

In some embodiments, the targeting ligand is an aptamer and the aptamer is selected from AS-1411, GB1-10, CGNKRTRGC (Lyp-1)(SEQ ID NO:1), F3 peptide, iRGD, KLWVLPKGGGC (SEQ ID NO:2), KLWVLPK (SEQ ID NO:3), and an aptide.

In some embodiments, the targeting ligand is a small molecule and the small molecule is selected from folate, folic acid, anisamide, phenylboronic acid, thiamine pyrophosphate (TPP), ((S)-2-(3-((S)-5-amino-1-carboxypentyl) ureido) pentanedioic acid (ACUPA), and 2-[3-(1, 3-dicarboxy propyl)-ureido] pentanedioic acid (DUPA).

In some embodiments, the targeting ligand is a nucleic acid-based ligand and the nucleic acid-based ligand is selected from A10 aptamer, and A9 CGA aptamer.

In some embodiments, the compound is a compound of Formula (II) and at least one R³ is H. In some embodiments, the compound is a compound of Formula (II) and at least one R³ is C₁₋₁₅ alkyl. In some embodiments, the compound is a compound of Formula (II) and at least one R³ is C₁₋₁₅ alkyl optionally substituted with one or more R¹⁰. In some embodiments, the compound is a compound of Formula (II) and at least one R³ is C₂₋₁₅ alkenyl. In some embodiments, the compound is a compound of Formula (II) and at least one R³ is C₂₋₁₅ alkenyl optionally substituted with one or more R¹⁰. In some embodiments, the compound is a compound of Formula (II) and at least one R³ is C₂₋₁₅ alkynyl. In some embodiments, the compound is a compound of Formula (II) and at least one R³ is C₂₋₁₅ alkynyl optionally substituted with one or more R¹⁰. In some embodiments, the compound is a compound of Formula (II) and at least one R³ is C₃₋₁₀ cycloalkyl. In some embodiments, the compound is a compound of Formula (II) and at least one R³ is C₃₋₁₀ cycloalkyl optionally substituted with one or more R¹⁰. In some embodiments, the compound is a compound of Formula (II) and at least one R³ is C₆₋₁₀ aryl. In some embodiments, the compound is a compound of Formula (II) and at least one R³ is C₆₋₁₀ aryl optionally substituted with one or more R¹⁰. In some embodiments, the compound is a compound of Formula (II) and at least one R³ is 5- to 10-membered heteroaryl. In some embodiments, the compound is a compound of Formula (II) and at least one R³ is 5- to 10-membered heteroaryl optionally substituted with one or more R¹⁰. In some embodiments, the compound is a compound of Formula (II) and at least one R³ is 4- to 10-membered heterocycloalkyl. In some embodiments, the compound is a compound of Formula (II) and at least one R³ is 4- to 10-membered heterocycloalkyl optionally substituted with one or more R¹⁰.

In some embodiments, the compound is a compound of Formula (II) and at least one R³ is selected from H, phenyl, pyridinyl,

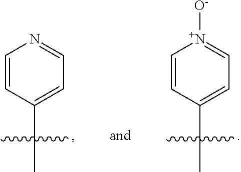 [SMILES: *c1ccncc1 ; *c1cc[n+]([O-])cc1]

In some embodiments, the compound is a compound of Formula (II) and at least one Ring B is C₆₋₁₀ aryl. In some embodiments, the compound is a compound of Formula (II) and at least one Ring B is phenyl. In some embodiments, the compound is a compound of Formula (II) and each Ring B is phenyl. In some embodiments, the compound is a compound of Formula (II) and at least one Ring B is 5- to 10-membered heteroaryl. In some embodiments, the compound is a compound of Formula (II) and at least one Ring B is pyridinyl. In some embodiments, the compound is a compound of Formula (II) and each Ring B is pyridinyl. In some embodiments, the compound is a compound of Formula (II) and at least one Ring B is thiophenyl. In some embodiments, the compound is a compound of Formula (II) and each Ring B is thiophenyl. In some embodiments, the compound is a compound of Formula (II) and at least one Ring B is furanyl. In some embodiments, the compound is a compound of Formula (II) and each Ring B is furanyl. In some embodiments, the compound is a compound of Formula (II) and at least one Ring B is pyrrolyl. In some embodiments, the compound is a compound of Formula (II) and each Ring B is pyrrolyl.

In some embodiments, the compound is a compound of Formula (II) and Ring C is C₆₋₁₀ aryl. In some embodiments, the compound is a compound of Formula (II) and Ring C is phenyl. In some embodiments, the compound is a compound of Formula (II) and Ring C is 5- to 10-membered heteroaryl. In some embodiments, the compound is a compound of Formula (II) and Ring C is pyridinyl. In some embodiments, the compound is a compound of Formula (II) and Ring C is thiophenyl. In some embodiments, the compound is a compound of Formula (II) and Ring C is furanyl. In some embodiments, the compound is a compound of Formula (II) and Ring C is pyrrolyl.

In some embodiments, the compound is a compound of Formula (II) and Ring D is C₆₋₁₀ aryl. In some embodiments, the compound is a compound of Formula (II) and Ring D is phenyl. In some embodiments, the compound is a compound of Formula (II) and Ring D is 5- to 10-membered heteroaryl. In some embodiments, the compound is a compound of Formula (II) and Ring D is pyridinyl. In some embodiments, the compound is a compound of Formula (II) and Ring D is thiophenyl. In some embodiments, the compound is a compound of Formula (II) and Ring D is furanyl. In some embodiments, the compound is a compound of Formula (II) and Ring D is pyrrolyl.

In some embodiments, the compound is a compound of Formula (II) and at least one Ring E is C₆₋₁₀ aryl. In some embodiments, the compound is a compound of Formula (II) and at least one Ring E is phenyl. In some embodiments, the compound is a compound of Formula (II) and each Ring E is phenyl. In some embodiments, the compound is a compound of Formula (II) and at least one Ring E is 5- to 10-membered heteroaryl. In some embodiments, the compound is a compound of Formula (II) and at least one Ring E is pyridinyl. In some embodiments, the compound is a compound of Formula (II) and each Ring E is pyridinyl. In some embodiments, the compound is a compound of Formula (II) and at least one Ring E is thiophenyl. In some embodiments, the compound is a compound of Formula (II) and each Ring E is thiophenyl. In some embodiments, the compound is a compound of Formula (II) and at least one Ring E is furanyl. In some embodiments, the compound is a compound of Formula (II) and each Ring E is furanyl. In some embodiments, the compound is a compound of Formula (II) and at least one Ring E is pyrrolyl. In some embodiments, the compound is a compound of Formula (II) and each Ring E is pyrrolyl.

In some embodiments, the compound is a compound of Formula (II) and at least one R⁴ is C₁₋₁₅ alkyl. In some embodiments, the compound is a compound of Formula (II) and at least one R⁴ is C₂₋₁₅ alkenyl. In some embodiments, the compound is a compound of Formula (II) and at least one R⁴ is C₂₋₁₅ alkynyl. In some embodiments, the compound is a compound of Formula (II) and at least one R⁴ is C₃₋₁₀ cycloalkyl. In some embodiments, the compound is a compound of Formula (II) and at least one R⁴ is C₆₋₁₀ aryl. In some embodiments, the compound is a compound of Formula (II) and at least one R⁴ is 5- to 10-membered heteroaryl. In some embodiments, the compound is a compound of Formula (II) and at least one R⁴ is 4- to 10-membered heterocycloalkyl. In some embodiments, the compound is a compound of Formula (II) and at least one R⁴ is halo. In some embodiments, the compound is a compound of Formula (II) and at least one R⁴ is —CN. In some embodiments, the compound is a compound of Formula (II) and at least one R⁴ is —OR^{O}. In some embodiments, the compound is a compound of Formula (II) and at least one R⁴ is —N(R^{N})₂. In some embodiments, the compound is a compound of Formula (II) and at least one R⁴ is —(C═O)R^{N}. In some embodiments, the compound is a compound of Formula (II) and at least one R⁴ is —(C═O)OR^{O}. In some embodiments, the compound is a compound of Formula (II) and at least one R⁴ is —(C═O)N(R^{N})₂. In some embodiments, the compound is a compound of Formula (II) and at least one R⁴ is —NR^{N}(C═O)R⁸. In some embodiments, the compound is a compound of Formula (II) and at least one R⁴ is —O(C═O)R⁸.

In some embodiments, the compound is a compound of Formula (II) and at least one R⁵ is C₁₋₁₅ alkyl. In some embodiments, the compound is a compound of Formula (II) and at least one R⁵ is C₂₋₁₅ alkenyl. In some embodiments, the compound is a compound of Formula (II) and at least one R⁵ is C₂₋₁₅ alkynyl. In some embodiments, the compound is a compound of Formula (II) and at least one R⁵ is C₃₋₁₀ cycloalkyl. In some embodiments, the compound is a compound of Formula (II) and at least one R⁵ is C₆₋₁₀ aryl. In some embodiments, the compound is a compound of Formula (II) and at least one R⁵ is 5- to 10-membered heteroaryl. In some embodiments, the compound is a compound of Formula (II) and at least one R⁵ is 4- to 10-membered heterocycloalkyl. In some embodiments, the compound is a compound of Formula (II) and at least one R⁵ is halo. In some embodiments, the compound is a compound of Formula (II) and at least one R⁵ is —CN. In some embodiments, the compound is a compound of Formula (II) and at least one R⁵ is —OR^{O}. In some embodiments, the compound is a compound of Formula (II) and at least one R⁵ is —N(R^{N})₂. In some embodiments, the compound is a compound of Formula (II) and at least one R⁵ is —(C═O)R^{N}. In some embodiments, the compound is a compound of Formula (II) and at least one R⁵ is —(C═O)OR^{O}. In some embodiments, the compound is a compound of Formula (II) and at least one R⁵ is —(C═O)N(R^{N})₂. In some embodiments, the compound is a compound of Formula (II) and at least one R⁵ is —NR^{N}(C═O)R⁸. In some embodiments, the compound is a compound of Formula (II) and at least one R⁵ is —O(C═O)R⁸.

In some embodiments, the compound is a compound of Formula (II) and at least one R⁶ is C₁₋₁₅ alkyl. In some embodiments, the compound is a compound of Formula (II) and at least one R⁶ is C₂₋₁₅ alkenyl. In some embodiments, the compound is a compound of Formula (II) and at least one R⁶ is C₂₋₁₅ alkynyl. In some embodiments, the compound is a compound of Formula (II) and at least one R⁶ is C₃₋₁₀ cycloalkyl. In some embodiments, the compound is a compound of Formula (II) and at least one R⁶ is C₆₋₁₀ aryl. In some embodiments, the compound is a compound of Formula (II) and at least one R⁶ is 5- to 10-membered heteroaryl. In some embodiments, the compound is a compound of Formula (II) and at least one R⁶ is 4- to 10-membered heterocycloalkyl. In some embodiments, the compound is a compound of Formula (II) and at least one R⁶ is halo. In some embodiments, the compound is a compound of Formula (II) and at least one R⁶ is —CN. In some embodiments, the compound is a compound of Formula (II) and at least one R⁶ is —OR^{O}. In some embodiments, the compound is a compound of Formula (II) and at least one R⁶ is —N(R^{N})₂. In some embodiments, the compound is a compound of Formula (II) and at least one R⁶ is —(C═O)R^{N}. In some embodiments, the compound is a compound of Formula (II) and at least one R⁶ is —(C═O)OR^{O}. In some embodiments, the compound is a compound of Formula (II) and at least one R⁶ is —(C═O)N(R^{N})₂. In some embodiments, the compound is a compound of Formula (II) and at least one R⁶ is —NR^{N}(C═O)R⁸. In some embodiments, the compound is a compound of Formula (II) and at least one R⁶ is —O(C═O)R⁸.

In some embodiments, the compound is a compound of Formula (II) and an R⁵ and an R⁶, together with the atoms to which they are attached, come together to form C₆₋₁₀ aryl. In some embodiments, the compound is a compound of Formula (II) and an R⁵ and an R⁶, together with the atoms to which they are attached, come together to form C₆₋₁₀ aryl, wherein the C₆₋₁₀ aryl is substituted with one or more R. In some embodiments, the compound is a compound of Formula (II) and an R⁵ and an R⁶, together with the atoms to which they are attached, come together to form 5- to 10-membered heteroaryl. In some embodiments, the compound is a compound of Formula (II) and an R⁵ and an R⁶, together with the atoms to which they are attached, come together to form 5- to 10-membered heteroaryl, wherein the 5- to 10-membered heteroaryl is substituted with one or more R⁸.

In some embodiments, the compound is a compound of Formula (II) and at least one R⁷ is C₁₋₁₅ alkyl. In some embodiments, the compound is a compound of Formula (II) and at least one R⁷ is C₂₋₁₅ alkenyl. In some embodiments, the compound is a compound of Formula (II) and at least one R⁷ is C₂₋₁₅ alkynyl. In some embodiments, the compound is a compound of Formula (II) and at least one R⁷ is C₃₋₁₀ cycloalkyl. In some embodiments, the compound is a compound of Formula (II) and at least one R⁷ is C₆₋₁₀ aryl. In some embodiments, the compound is a compound of Formula (II) and at least one R⁷ is 5- to 10-membered heteroaryl. In some embodiments, the compound is a compound of Formula (II) and at least one R⁷ is 4- to 10-membered heterocycloalkyl. In some embodiments, the compound is a compound of Formula (II) and at least one R⁷ is halo. In some embodiments, the compound is a compound of Formula (II) and at least one R⁷ is —CN. In some embodiments, the compound is a compound of Formula (II) and at least one R⁷ is —OR^{O}. In some embodiments, the compound is a compound of Formula (II) and at least one R⁷ is —N(R^{N})₂. In some embodiments, the compound is a compound of Formula (II) and at least one R⁷ is —(C═O)R^{N}. In some embodiments, the compound is a compound of Formula (II) and at least one R⁷ is —(C═O)OR^{O}. In some embodiments, the compound is a compound of Formula (II) and at least one R⁷ is —(C═O)N(R^{N})₂. In some embodiments, the compound is a compound of Formula (II) and at least one R⁷ is —NR^{N}(C═O)R⁸. In some embodiments, the compound is a compound of Formula (II) and at least one R⁷ is —O(C═O)R⁸.

In some embodiments, the compound is a compound of Formula (II) and at least one R⁸ is H. In some embodiments, the compound is a compound of Formula (II) and at least one R⁸ is C₁₋₁₅ alkyl. In some embodiments, the compound is a compound of Formula (II) and at least one R⁸ is C₂₋₁₅ alkenyl. In some embodiments, the compound is a compound of Formula (II) and at least one R⁸ is C₂₋₁₅ alkynyl.

In some embodiments, the compound is a compound of Formula (II) and at least one R⁹ is H. In some embodiments, the compound is a compound of Formula (II) and at least one R⁹ is C₁₋₁₅ alkyl. In some embodiments, the compound is a compound of Formula (II) and at least one R⁹ is C₂₋₁₅ alkenyl. In some embodiments, the compound is a compound of Formula (II) and at least one R⁹ is C₂₋₁₅ alkynyl. In some embodiments, the compound is a compound of Formula (II) and two R⁹, together with the N atom to which they are attached, come together to form 4- to 10-membered heterocycloalkyl optionally substituted with one or more oxo. In some embodiments, the compound is a compound of Formula (II) and two R⁹, together with the N atom to which they are attached, come together to form 5-membered heterocycloalkyl substituted with one or more oxo.

In some embodiments, the compound is a compound of Formula (II) and at least one R¹⁰ is C₁₋₁₀₀ alkyl. In some embodiments, the compound is a compound of Formula (II) and at least one R¹⁰ is C₂₋₁₀₀ alkenyl. In some embodiments, the compound is a compound of Formula (II) and at least one R¹⁰ is C₂₋₁₀₀ alkynyl. In some embodiments, the compound is a compound of Formula (II) and at least one R¹⁰ is C₁₋₁₀₀ haloalkyl. In some embodiments, the compound is a compound of Formula (II) and at least one R¹⁰ is halo. In some embodiments, the compound is a compound of Formula (II) and at least one R¹⁰ is —CN. In some embodiments, the compound is a compound of Formula (II) and at least one R¹⁰ is —OR^{O}. In some embodiments, the compound is a compound of Formula (II) and at least one R¹⁰ is oxo. In some embodiments, the compound is a compound of Formula (II) and at least one R¹⁰ is —N(R^{N})₂. In some embodiments, the compound is a compound of Formula (II) and at least one R¹⁰ is —(C═O)R^{N}. In some embodiments, the compound is a compound of Formula (II) and at least one R¹⁰ is —(C═O)OR^{O}. In some embodiments, the compound is a compound of Formula (II) and at least one R¹⁰ is —(C═O)N(R^{N})₂. In some embodiments, the compound is a compound of Formula (II) and at least one R¹⁰ is —NR^{N}(C═O)R⁸. In some embodiments, the compound is a compound of Formula (II) and at least one R¹⁰ is —O(C═O)R⁸.

In some embodiments, the compound is a compound of Formula (II) and an R⁵ and an R¹⁰, together with the atoms to which they are attached, come together to form C₆₋₁₀ aryl. In some embodiments, the compound is a compound of Formula (II) and an R⁵ and an R¹⁰, together with the atoms to which they are attached, come together to form C₆₋₁₀ aryl, wherein the C₆₋₁₀ aryl or 5- to 10-membered heteroaryl is optionally substituted with one or more R. In some embodiments, the compound is a compound of Formula (II) and an R⁵ and an R¹⁰, together with the atoms to which they are attached, come together to form 5- to 10-membered heteroaryl. In some embodiments, the compound is a compound of Formula (II) and an R⁵ and an R¹⁰, together with the atoms to which they are attached, come together to form 5- to 10-membered heteroaryl, wherein the C₆₋₁₀ aryl or 5- to 10-membered heteroaryl is optionally substituted with one or more R⁸.

In some embodiments, the compound is a compound of Formula (II) and an R⁶ and an R¹⁰, together with the atoms to which they are attached, come together to form C₆₋₁₀ aryl. In some embodiments, the compound is a compound of Formula (II) and an R⁶ and an R¹⁰, together with the atoms to which they are attached, come together to form C₆₋₁₀ aryl, wherein the C₆₋₁₀ aryl is substituted with one or more R⁸. In some embodiments, the compound is a compound of Formula (II) and an R⁶ and an R¹⁰, together with the atoms to which they are attached, come together to form 5- to 10-membered heteroaryl. In some embodiments, the compound is a compound of Formula (II) and an R⁶ and an R¹⁰, together with the atoms to which they are attached, come together to form 5- to 10-membered heteroaryl, wherein the 5- to 10-membered heteroaryl is substituted with one or more R⁸.

In some embodiments, the compound is a compound of Formula (II) and at least one R¹¹ is C₁₋₁₅ alkyl. In some embodiments, the compound is a compound of Formula (II) and at least one R¹¹ is C₁₋₁₅ alkyl substituted with one or more R¹². In some embodiments, the compound is a compound of Formula (II) and at least one R¹¹ is C₂₋₁₅ alkenyl. In some embodiments, the compound is a compound of Formula (II) and at least one R¹¹ is C₂₋₁₅ alkenyl substituted with one or more R¹². In some embodiments, the compound is a compound of Formula (II) and at least one R¹¹ is C₂₋₁₅ alkynyl. In some embodiments, the compound is a compound of Formula (II) and at least one R¹¹ is C₂₋₁₅ alkynyl substituted with one or more R¹².

In some embodiments, the compound is a compound of Formula (II) and at least one R² is C₃₋₁₀ cycloalkyl. In some embodiments, the compound is a compound of Formula (II) and at least one R¹² is C₃₋₁₀ cycloalkyl optionally substituted with one or more C₁₋₁₅ alkyl, C₂₋₁₅ alkenyl, and C₂₋₁₅ alkynyl, halo, —CN, —OR^{O}, and —N(R^{N})₂. In some embodiments, the compound is a compound of Formula (II) and at least one R¹² is C₆₋₁₀ aryl. In some embodiments, the compound is a compound of Formula (II) and at least one R¹² is C₆₋₁₀ aryl optionally substituted with one or more C₁₋₁₅ alkyl, C₂₋₁₅ alkenyl, and C₂₋₁₅ alkynyl, halo, —CN, —OR^{O}, and —N(R^{N})₂. In some embodiments, the compound is a compound of Formula (II) and at least one R¹² is 5- to 10-membered heteroaryl. In some embodiments, the compound is a compound of Formula (II) and at least one R¹² is 5- to 10-membered heteroaryl optionally substituted with one or more C₁₋₁₅ alkyl, C₂₋₁₅ alkenyl, and C₂₋₁₅ alkynyl, halo, —CN, —OR^{O}, and —N(R^{N})₂. In some embodiments, the compound is a compound of Formula (II) and at least one R¹² is 4- to 10-membered heterocycloalkyl. In some embodiments, the compound is a compound of Formula (II) and at least one R¹² is 4- to 10-membered heterocycloalkyl optionally substituted with one or more C₁₋₁₅ alkyl, C₂₋₁₅ alkenyl, and C₂₋₁₅ alkynyl, halo, —CN, —OR^{O}, and —N(R^{N})₂. In some embodiments, the compound is a compound of Formula (II) and at least one R¹² is halo. In some embodiments, the compound is a compound of Formula (II) and at least one R¹² is —CN. In some embodiments, the compound is a compound of Formula (II) and at least one R¹² is —OR^{O}. In some embodiments, the compound is a compound of Formula (II) and at least one R¹² is —N(R^{N})₂. In some embodiments, the compound is a compound of Formula (II) and at least one R¹² is —(C═O)R^{N}. In some embodiments, the compound is a compound of Formula (II) and at least one R¹² is —(C═O)OR^{O}. In some embodiments, the compound is a compound of Formula (II) and at least one R¹² is —(C═O)N(R^{N})₂. In some embodiments, the compound is a compound of Formula (II) and at least one R¹² is —NR^{N}(C═O)R⁸. In some embodiments, the compound is a compound of Formula (II) and at least one R¹² is —NR^{N}(C═NR^{N})R^{N}. In some embodiments, the compound is a compound of Formula (II) and at least one R¹² is —O(C═O)R⁸. In some embodiments, the compound is a compound of Formula (II) and at least one R¹² is —SR⁸.

In some embodiments, the compound is a compound of Formula (II) and at least one R^{N} is H. In some embodiments, the compound is a compound of Formula (II) and at least one R^{N} is C₁₋₁₅ alkyl. In some embodiments, the compound is a compound of Formula (II) and at least one R^{N} is C₁₋₁₅ alkyl optionally substituted with one or more substituents independently selected from the group consisting of halo, —CN, and —OR^{O}. In some embodiments, the compound is a compound of Formula (II) and at least one R^{N} is C₂₋₁₅ alkenyl. In some embodiments, the compound is a compound of Formula (II) and at least one R^{N} is C₂₋₁₅ alkenyl optionally substituted with one or more substituents independently selected from the group consisting of halo, —CN, and —OR^{O}. In some embodiments, the compound is a compound of Formula (II) and at least one R^{N} is C₂₋₁₅ alkynyl. In some embodiments, the compound is a compound of Formula (II) and at least one R^{N} is C₂₋₁₅ alkynyl optionally substituted with one or more substituents independently selected from the group consisting of halo, —CN, and —OR^{O}.

In some embodiments, the compound is a compound of Formula (II) and at least one R^{O} is C₁₋₁₅ alkyl optionally substituted with one or more substituents independently selected from the group consisting of halo and —CN. In some embodiments, the compound is a compound of Formula (II) and at least one R^{O} is C₂₋₁₅ alkenyl. In some embodiments, the compound is a compound of Formula (II) and at least one R^{O} is C₂₋₁₅ alkenyl optionally substituted with one or more substituents independently selected from the group consisting of halo and —CN. In some embodiments, the compound is a compound of Formula (II) and at least one R^{O} is C₂₋₁₅ alkynyl. In some embodiments, the compound is a compound of Formula (II) and at least one R^{O} is C₂₋₁₅ alkynyl optionally substituted with one or more substituents independently selected from the group consisting of halo and —CN.

In some embodiments, the compound is a compound of Formula (II) and m is 1. In some embodiments, the compound is a compound of Formula (II) and m is 2. In some embodiments, the compound is a compound of Formula (II) and m is 3. In some embodiments, the compound is a compound of Formula (II) and m is 4. In some embodiments, the compound is a compound of Formula (II) and m is 1. In some embodiments, the compound is a compound of Formula (II) and m is 5.

In some embodiments, the compound is a compound of Formula (II) and n is 0. In some embodiments, the compound is a compound of Formula (II) and n is 1. In some embodiments, the compound is a compound of Formula (II) and n is 2. In some embodiments, the compound is a compound of Formula (II) and n is 3. In some embodiments, the compound is a compound of Formula (II) and n is 4.

In some embodiments, the compound is a compound of Formula (II) and p is 0. In some embodiments, the compound is a compound of Formula (II) and p is 1. In some embodiments, the compound is a compound of Formula (II) and p is 2. In some embodiments, the compound is a compound of Formula (II) and p is 3. In some embodiments, the compound is a compound of Formula (II) and p is 4.

In some embodiments, the compound is a compound of Formula (II) and q is an integer selected from 1 to 2500. In some embodiments, the compound is a compound of Formula (II) and q is an integer selected from 1 to 2000. In some embodiments, the compound is a compound of Formula (II) and q is an integer selected from 1 to 1500. In some embodiments, the compound is a compound of Formula (II) and q is an integer selected from 1 to 1000. In some embodiments, the compound is a compound of Formula (II) and q is an integer selected from 1 to 500. In some embodiments, the compound is a compound of Formula (II) and q is an integer selected from 500 to 2500. In some embodiments, the compound is a compound of Formula (II) and q is an integer selected from 1000 to 2500. In some embodiments, the compound is a compound of Formula (II) and q is an integer selected from 1500 to 2500. In some embodiments, the compound is a compound of Formula (II) and q is an integer selected from 2000 to 2500. In some embodiments, the compound is a compound of Formula (II) and q is an integer selected from 500 to 2000. In some embodiments, the compound is a compound of Formula (II) and q is an integer selected from 1000 to 1500.

In some embodiments, the compound is a compound of Formula (II) and s is 0. In some embodiments, the compound is a compound of Formula (II) and s is 1. In some embodiments, the compound is a compound of Formula (II) and s is 2. In some embodiments, the compound is a compound of Formula (II) and s is 3. In some embodiments, the compound is a compound of Formula (II) and s is 4. In some embodiments, the compound is a compound of Formula (II) and s is 5.

In some embodiments, the compound is a compound of Formula (II) and t is 0. In some embodiments, the compound is a compound of Formula (II) and t is 1. In some embodiments, the compound is a compound of Formula (II) and t is 2. In some embodiments, the compound is a compound of Formula (II) and t is 3. In some embodiments, the compound is a compound of Formula (II) and t is 4. In some embodiments, the compound is a compound of Formula (II) and s is 5.

In some embodiments, the compound is a compound of Formula (II) and w is 0. In some embodiments, the compound is a compound of Formula (II) and w is 1. In some embodiments, the compound is a compound of Formula (II) and w is 2. In some embodiments, the compound is a compound of Formula (II) and w is 3. In some embodiments, the compound is a compound of Formula (II) and w is 4.

In some embodiments, the compound is a compound of Formula (II) and x is 0. In some embodiments, the compound is a compound of Formula (II) and x is 1. In some embodiments, the compound is a compound of Formula (II) and x is 2. In some embodiments, the compound is a compound of Formula (II) and x is 3. In some embodiments, the compound is a compound of Formula (II) and x is 4.

In some embodiments, the compound is a compound of Formula (II) and y is 0. In some embodiments, the compound is a compound of Formula (II) and y is 1. In some embodiments, the compound is a compound of Formula (II) and y is 2. In some embodiments, the compound is a compound of Formula (II) and y is 3. In some embodiments, the compound is a compound of Formula (II) and y is 4.

In some embodiments, the compound is a compound of Formula (II) and z is 0. In some embodiments, the compound is a compound of Formula (II) and z is 1. In some embodiments, the compound is a compound of Formula (II) and z is 2. In some embodiments, the compound is a compound of Formula (II) and z is 3. In some embodiments, the compound is a compound of Formula (II) and z is 4.

At various places in the present specification, certain features of the compounds are disclosed in groups or in ranges. It is specifically intended that such a disclosure include each and every individual subcombination of the members of such groups and ranges. For example, the term “C₁₋₆ alkyl” is specifically intended to individually disclose (without limitation) methyl, ethyl, C₃ alkyl, C₄ alkyl, C₅ alkyl and C₆ alkyl.

The term “n-membered,” where n is an integer, typically describes the number of ring-forming atoms in a moiety where the number of ring-forming atoms is n. For example, piperidinyl is an example of a 6-membered heterocycloalkyl ring, pyrazolyl is an example of a 5-membered heteroaryl ring, pyridyl is an example of a 6-membered heteroaryl ring and 1,2,3,4-tetrahydro-naphthalene is an example of a 10-membered cycloalkyl group.

The term “substituted” means that an atom or group of atoms formally replaces hydrogen as a “substituent” attached to another group. The term “substituted”, unless otherwise indicated, refers to any level of substitution, e.g., mono-, di-, tri-, tetra- or penta-substitution, where such substitution is permitted. The substituents are independently selected, and substitution may be at any chemically accessible position. It is to be understood that substitution at a given atom is limited by valency. It is to be understood that substitution at a given atom results in a chemically stable molecule. The phrase “optionally substituted” means unsubstituted or substituted. The term “substituted” means that a hydrogen atom is removed and replaced by a substituent. A single divalent substituent, e.g., oxo, can replace two hydrogen atoms.

The term “Cₙ₋ₘ” indicates a range which includes the endpoints, wherein n and m are integers and indicate the number of carbons. Examples include C₁₋₄, C₁₋₆ and the like.

The term “alkyl” employed alone or in combination with other terms, refers to a saturated hydrocarbon group that may be straight-chained or branched. The term “Cₙ₋ₘ alkyl”, refers to an alkyl group having n to m carbon atoms. An alkyl group formally corresponds to an alkane with one C—H bond replaced by the point of attachment of the alkyl group to the remainder of the compound. Examples of alkyl moieties include, but are not limited to, chemical groups such as methyl, ethyl, n-propyl, isopropyl, n-butyl, tert-butyl, isobutyl, sec-butyl; higher homologs such as 2-methyl-1-butyl, n-pentyl, 3-pentyl, n-hexyl, 1,2,2-trimethylpropyl and the like.

The term “alkenyl” employed alone or in combination with other terms, refers to a straight-chain or branched hydrocarbon group corresponding to an alkyl group having one or more double carbon-carbon bonds. An alkenyl group formally corresponds to an alkene with one C—H bond replaced by the point of attachment of the alkenyl group to the remainder of the compound. The term “Cₙ₋ₘ alkenyl” refers to an alkenyl group having n to m carbons. Example alkenyl groups include, but are not limited to, ethenyl, n-propenyl, isopropenyl, n-butenyl, sec-butenyl and the like.

The term “alkynyl” employed alone or in combination with other terms, refers to a straight-chain or branched hydrocarbon group corresponding to an alkyl group having one or more triple carbon-carbon bonds. An alkynyl group formally corresponds to an alkyne with one C—H bond replaced by the point of attachment of the alkyl group to the remainder of the compound. The term “Cₙ₋ₘ alkynyl” refers to an alkynyl group having n to m carbons. Example alkynyl groups include, but are not limited to, ethynyl, propyn-1-yl, propyn-2-yl and the like.

The term “haloalkyl” as used herein refers to an alkyl group in which one or more of the hydrogen atoms has been replaced by a halogen atom. The term “Cₙ₋ₘ haloalkyl” refers to a Cₙ₋ₘ alkyl group having n to m carbon atoms and from at least one up to {2(n to m)+1}halogen atoms, which may either be the same or different. In some embodiments, the halogen atoms are fluoro atoms. Example haloalkyl groups include CF₃, C₂F₅, CHF₂, CCl₃, CHCl₂, C₂Cl₅ and the like. In some embodiments, the haloalkyl group is a fluoroalkyl group. In some embodiments, the haloalkyl group is a chloroalkyl group. In some embodiments, the haloalkyl group is a bromoalkyl group. In some embodiments, the haloalkyl group is a iodoalkyl group.

The term “cyano” or “nitrile” refers to a group of formula —C≡N, which also may be written as —CN.

The terms “halo” or “halogen”, used alone or in combination with other terms, refers to fluoro, chloro, bromo and iodo. In some embodiments, “halo” refers to a halogen atom selected from F, Cl, or Br.

The term “oxo” refers to an oxygen atom as a divalent substituent, forming a carbonyl group when attached to carbon, or attached to a heteroatom forming a sulfoxide or sulfone group, or an N-oxide group. In some embodiments, heterocyclic groups may be optionally substituted by 1 or 2 oxo (═O) substituents.

The term “aryl,” employed alone or in combination with other terms, refers to an aromatic hydrocarbon group, which may be monocyclic or polycyclic (e.g., having 2 fused rings). The term “Cₙ₋ₘ aryl” refers to an aryl group having from n to m ring carbon atoms. Aryl groups include, e.g., phenyl, naphthyl, indanyl, indenyl and the like. In some embodiments, aryl groups have from 6 to about 10 carbon atoms. In some embodiments aryl groups have 6 carbon atoms. In some embodiments aryl groups have 10 carbon atoms. In some embodiments, the aryl group is phenyl. In some embodiments, the aryl group is naphthyl.

The term “heteroatom” used herein is meant to include boron, phosphorus, sulfur, oxygen and nitrogen.

The term “heteroaryl” or “heteroaromatic,” employed alone or in combination with other terms, refers to a monocyclic or polycyclic aromatic heterocycle having at least one heteroatom ring member selected from boron, phosphorus, sulfur, oxygen and nitrogen. In some embodiments, the heteroaryl ring has 1, 2, 3 or 4 heteroatom ring members independently selected from nitrogen, sulfur and oxygen. In some embodiments, any ring-forming N in a heteroaryl moiety can be an N-oxide. In some embodiments, the heteroaryl has 5-14 ring atoms including carbon atoms and 1, 2, 3 or 4 heteroatom ring members independently selected from nitrogen, sulfur and oxygen. In some embodiments, the heteroaryl has 5-14, or 5-10 ring atoms including carbon atoms and 1, 2, 3 or 4 heteroatom ring members independently selected from nitrogen, sulfur and oxygen. In some embodiments, the heteroaryl has 5-6 ring atoms and 1 or 2 heteroatom ring members independently selected from nitrogen, sulfur and oxygen. In some embodiments, the heteroaryl is a five-membered or six-membered heteroaryl ring. In other embodiments, the heteroaryl is an eight-membered, nine-membered or ten-membered fused bicyclic heteroaryl ring. Example heteroaryl groups include, but are not limited to, pyridinyl (pyridyl), pyrimidinyl, pyrazinyl, pyridazinyl, pyrrolyl, pyrazolyl, azolyl, oxazolyl, thiazolyl, imidazolyl, furanyl, thiophenyl, quinolinyl, isoquinolinyl, naphthyridinyl (including 1,2-, 1,3-, 1,4-, 1,5-, 1,6-, 1,7-, 1,8-, 2,3- and 2,6-naphthyridine), indolyl, benzothiophenyl, benzofuranyl, benzisoxazolyl, imidazo[1,2-b]thiazolyl, purinyl, and the like.

A five-membered heteroaryl ring is a heteroaryl group having five ring atoms wherein one or more (e.g., 1, 2 or 3) ring atoms are independently selected from N, O and S. Exemplary five-membered ring heteroaryls include thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl and 1,3,4-oxadiazolyl.

A six-membered heteroaryl ring is a heteroaryl group having six ring atoms wherein one or more (e.g., 1, 2 or 3) ring atoms are independently selected from N, O and S. Exemplary six-membered ring heteroaryls are pyridyl, pyrazinyl, pyrimidinyl, triazinyl and pyridazinyl.

The term “cycloalkyl,” employed alone or in combination with other terms, refers to a non-aromatic hydrocarbon ring system (monocyclic, bicyclic or polycyclic), including cyclized alkyl and alkenyl groups. The term “Cₙ₋ₘ cycloalkyl” refers to a cycloalkyl that has n to m ring member carbon atoms. Cycloalkyl groups can include mono- or polycyclic (e.g., having 2, 3 or 4 fused rings) groups and spirocycles. Cycloalkyl groups can have 3, 4, 5, 6, 7, 8, 9, 10, 11, 12, 13, or 14 ring-forming carbons (C₃₋₁₄). In some embodiments, the cycloalkyl group has 3 to 14 members, 3 to 10 members, 3 to 6 ring members, 3 to 5 ring members, or 3 to 4 ring members. In some embodiments, the cycloalkyl group is monocyclic. In some embodiments, the cycloalkyl group is monocyclic or bicyclic. In some embodiments, the cycloalkyl group is a C₃₋₆ monocyclic cycloalkyl group. Ring-forming carbon atoms of a cycloalkyl group can be optionally oxidized to form an oxo or sulfido group. Cycloalkyl groups also include cycloalkylidenes. In some embodiments, cycloalkyl is cyclopropyl, cyclobutyl, cyclopentyl or cyclohexyl. Also included in the definition of cycloalkyl are moieties that have one or more aromatic rings fused (i.e., having a bond in common with) to the cycloalkyl ring, e.g., benzo or thienyl derivatives of cyclopentane, cyclohexane and the like. A cycloalkyl group containing a fused aromatic ring can be attached through any ring-forming atom including a ring-forming atom of the fused aromatic ring. Examples of cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, bicyclo[1.1.1]pentanyl, bicyclo[2.1.1]hexanyl, and the like. In some embodiments, the cycloalkyl group is cyclopropyl, cyclobutyl, cyclopentyl, or cyclohexyl.

The term “heterocycloalkyl,” employed alone or in combination with other terms, refers to a non-aromatic ring or ring system, which may optionally contain one or more alkenylene groups as part of the ring structure, which has at least one heteroatom ring member independently selected from boron, nitrogen, sulfur oxygen and phosphorus, and which has 4-14 ring members, 4-10 ring members, 4-7 ring members, or 4-6 ring members. Included within the term “heterocycloalkyl” are monocyclic 4-, 5-, 6- and 7-membered heterocycloalkyl groups. Heterocycloalkyl groups can include mono- or bicyclic or polycyclic (e.g., having two or three fused or bridged rings) ring systems or spirorcycles. In some embodiments, the heterocycloalkyl group is a monocyclic group having 1, 2 or 3 heteroatoms independently selected from nitrogen, sulfur and oxygen. Ring-forming carbon atoms and heteroatoms of a heterocycloalkyl group can be optionally oxidized to form an oxo or sulfido group or other oxidized linkage (e.g., C(O), S(O), C(S) or S(O)₂, N-oxide etc.) or a nitrogen atom can be quaternized. The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds. Also included in the definition of heterocycloalkyl are moieties that have one or more aromatic rings fused (i.e., having a bond in common with) to the heterocycloalkyl ring, e.g., benzo or thienyl derivatives of piperidine, morpholine, azepine, etc. A heterocycloalkyl group containing a fused aromatic ring can be attached through any ring-forming atom including a ring-forming atom of the fused aromatic ring. Examples of heterocycloalkyl groups include azetidinyl, azepanyl, dihydrobenzofuranyl, dihydrofuranyl, dihydropyranyl, morpholino, 3-oxa-9-azaspiro[5.5]undecanyl, 1-oxa-8-azaspiro[4.5]decanyl, piperidinyl, piperazinyl, oxopiperazinyl, pyranyl, pyrrolidinyl, quinuclidinyl, tetrahydrofuranyl, tetrahydropyranyl, 1,2,3,4-tetrahydroquinolinyl, tropanyl, 4,5,6,7-tetrahydrothiazolo[5,4-c]pyridinyl, and thiomorpholino.

At certain places, the definitions or embodiments refer to specific rings (e.g., an azetidine ring, a pyridine ring, etc.). Unless otherwise indicated, these rings can be attached to any ring member provided that the valency of the atom is not exceeded. For example, an azetidine ring may be attached at any position of the ring, whereas an azetidin-3-yl ring is attached at the 3-position.

The compounds described herein can be asymmetric (e.g., having one or more stereocenters). All stereoisomers, such as enantiomers and diastereomers, are intended unless otherwise indicated. Compounds of the present invention that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Many geometric isomers of olefins, C═N double bonds and the like can also be present in the compounds described herein, and all such stable isomers are contemplated in the present invention. Cis and trans geometric isomers of the compounds of the present invention are described and may be isolated as a mixture of isomers or as separated isomeric forms.

Resolution of racemic mixtures of compounds can be carried out by any of numerous methods known in the art. One method includes fractional recrystallization using a chiral resolving acid which is an optically active, salt-forming organic acid. Suitable resolving agents for fractional recrystallization methods are, e.g., optically active acids, such as the D and L forms of tartaric acid, diacetyltartaric acid, dibenzoyltartaric acid, mandelic acid, malic acid, lactic acid or the various optically active camphorsulfonic acids such as 3-camphorsulfonic acid. Other resolving agents suitable for fractional crystallization methods include stereoisomerically pure forms of α-methylbenzylamine (e.g., S and R forms, or diastereomerically pure forms), 2-phenylglycinol, norephedrine, ephedrine, N-methylephedrine, cyclohexylethylamine, 1,2-diaminocyclohexane and the like.

Resolution of racemic mixtures can also be carried out by elution on a column packed with an optically active resolving agent (e.g., dinitrobenzoylphenylglycine). Suitable elution solvent composition can be determined by one skilled in the art.

In some embodiments, the compounds of the invention have the (R)-configuration. In other embodiments, the compounds have the (S)-configuration. In compounds with more than one chiral centers, each of the chiral centers in the compound may be independently (R) or (S), unless otherwise indicated.

Compounds of the invention also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, enamine-imine pairs, and annular forms where a proton can occupy two or more positions of a heterocyclic system, e.g., 1H- and 3H-imidazole, 1H-, 2H- and 4H-1,2,4-triazole, 1H- and 2H-isoindole and 1H- and 2H-pyrazole. Tautomeric forms can be in equilibrium or sterically locked into one form by appropriate substitution.

Compounds of the invention can also include all isotopes of atoms occurring in the intermediates or final compounds. Isotopes include those atoms having the same atomic number but different mass numbers. For example, isotopes of hydrogen include tritium and deuterium. One or more constituent atoms of the compounds of the invention can be replaced or substituted with isotopes of the atoms in natural or non-natural abundance. In some embodiments, the compound includes at least one deuterium atom. For example, one or more hydrogen atoms in a compound of the present disclosure can be replaced or substituted by deuterium. In some embodiments, the compound includes two or more deuterium atoms. In some embodiments, the compound includes 1, 2, 3, 4, 5, 6, 7, 8, 9, 10, 11 or 12 deuterium atoms. Synthetic methods for including isotopes into organic compounds are known in the art.

The term, “compound,” as used herein is meant to include all stereoisomers, geometric isomers, tautomers and isotopes of the structures depicted. The term is also meant to refer to compounds of the inventions, regardless of how they are prepared, e.g., synthetically, through biological process (e.g., metabolism or enzyme conversion), or a combination thereof.

Also provided herein are compositions comprising the compounds. In some embodiments, the compositions are particles, e.g., nanoparticles, microspheres, liposomes, or other particles, preferably wherein the compounds are incorporated into the surface of the particles (e.g., in a membrane, e.g., monolayer, bilayer, or trilayer), surrounding the particles).

Particles may be nanoparticles. Nanoparticles are preferred for intertissue application, penetration of cells, and certain routes of administration. The nanoparticles may have any desired size for the intended use. The nanoparticles may have any diameter from 10 nm to 1,000 nm. The nanoparticle can have a diameter from 10 nm to 900 nm, from 10 nm to 800 nm, from 10 nm to 700 nm, from 10 nm to 600 nm, from 10 nm to 500 nm, from 20 nm from 500 nm, from 30 nm to 500 nm, from 40 nm to 500 nm, from 50 nm to 500 nm, from 50 nm to 400 nm, from 50 nm to 350 nm, from 50 nm to 300 nm, or from 50 nm to 200 nm. In preferred embodiments the nanoparticles can have a diameter less than 400 nm, less than 300 nm, or less than 200 nm. The preferred range is between 10 nm and 300 nm.

The particles can be polymeric particles, non-polymeric particles (e.g., a metal particle, quantum dot, ceramic, inorganic material, bone, etc.), liposomes, micelles, polymeric micelles, viral particles, hybrids thereof, and/or combinations thereof. In some embodiments, the particles are, but not limited to, one or a plurality of lipid-based nanoparticles, polymeric nanoparticles, metallic nanoparticles, surfactant-based emulsions, dendrimers, buckyballs, nanowires, virus-like particles, peptide or protein-based particles (such as albumin nanoparticles) and/or nanoparticles that are developed using a combination of nanomaterials such as lipid-polymer nanoparticles. In some embodiments, nanoparticles can comprise one or more polymers.

Nanoparticles may be a variety of different shapes, including but not limited to spheroidal, cubic, pyramidal, oblong, cylindrical, toroidal, and the like. Nanoparticles can comprise one or more surfaces. Exemplary nanoparticles that can be adapted for use include (1) the biodegradable nanoparticles disclosed in U.S. Pat. No. 5,543,158 to Gref et al., (2) the polymeric nanoparticles of Published US Patent Application 20060002852 to Saltzman et al., or (4) the lithographically constructed nanoparticles of Published US Patent Application 20090028910 to DeSimone et al.

In some embodiments the nanoparticles are configured with a core and envelope structure, wherein the envelope comprises a surface membrane or layer surrounding the core, e.g., a monolayer or bilayer, preferably wherein the lipid-PEG compositions are incorporated into the surface layer.

In some embodiments, the particles comprise at least 0.1%, 1%, 5%, 10%, 15%, 20%, 25%, 30%, 35%, 40%, 50%, 60%, 70%, 80%, 90%, 95%, or 100% by weight of the lipid-PEG compounds as described herein. In some embodiments, the particles comprise at least 0.1% and up to 1%, 5%, 10%, 15%, 20%, 25%, 30%, 35%, 40%, 50%, 60%, 70%, 80%, 90%, 95%, or 100% by weight of the lipid-PEG compounds as described herein, or any range with end points of 0.1%, 1%, 5%, 10%, 15%, 20%, 25%, 30%, 35%, 40%, 50%, 60%, 70%, 80%, 90%, 95%, or 100% by weight.

In some embodiments, the compositions (e.g., particles) further comprise one or more lipids, e.g., ionizable lipids such as G0-Cm (e.g., G0-C8) DLin-MC3-DMA ((6Z,9Z,28Z,31Z)-heptatriacont-6,9,28,31-tetraene-19-yl 4-(dimethylamino)butanoate (also referred to herein as MC3)), SM-102, ALC-0315, multi-tailed ionizable phospholipids (iPhos)³¹, or others (see Chem. Rev. 2021, 121, 20, 12181-12277)¹⁰; phospholipids such as DOPE or DSPC; and cholesterol or its analogues (e.g., any other cholestanoid, i.e., any steroid based on a cholestane skeleton and its derivatives, e.g., C27 bile acids). Additional lipids can also be included such as dioleoyl-3-trimethylammonium propane (DOTAP), 1,2-di-O-octadecenyl-3-trimethylammonium propane (DOTMA), DDAB, DODAP, EPC, 18BMP, and others²³.

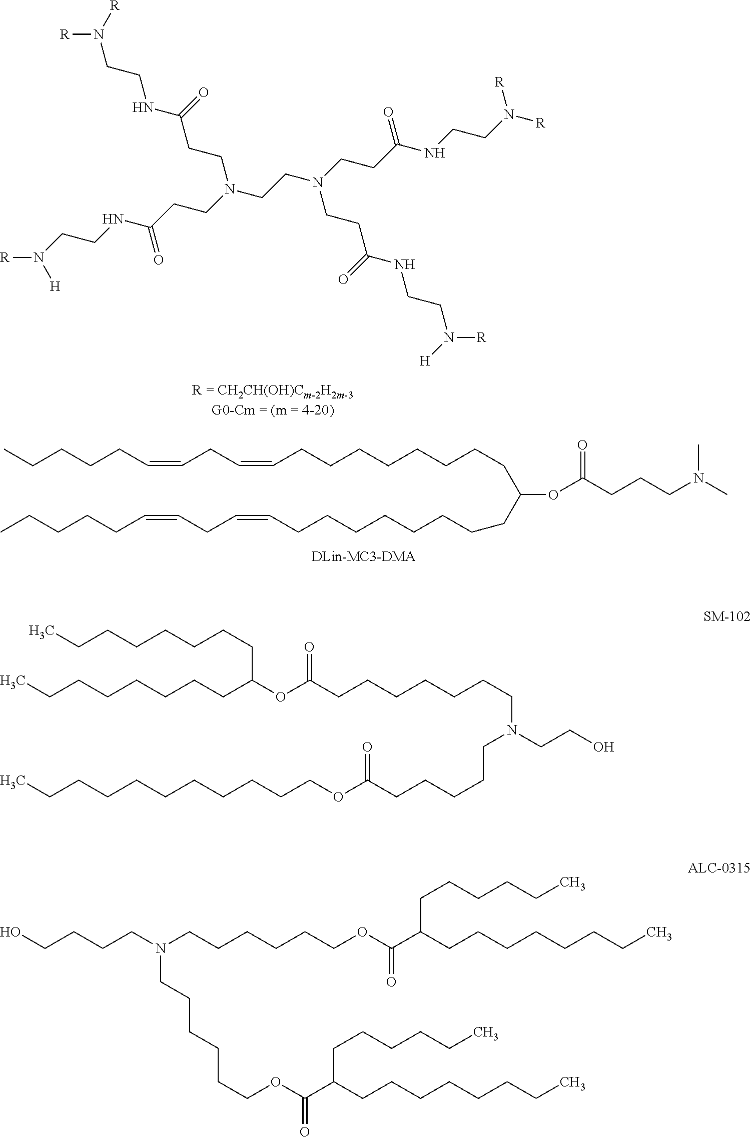

1. Lipid-Based Particles

In some embodiments, the nanoparticles can optionally further comprise one or more lipids. In some embodiments, a nanoparticle may comprise a liposome. In some embodiments, a nanoparticle may comprise a lipid bilayer. In some embodiments, a nanoparticle may comprise a lipid monolayer. In some embodiments, a nanoparticle may comprise a micelle. In some embodiments, a nanoparticle may comprise a core comprising a polymeric matrix surrounded by a lipid layer (e.g., lipid bilayer, lipid monolayer, etc.) comprising the lipid-PEG compounds as described herein. In some embodiments, a nanoparticle may comprise a non-polymeric core (e.g., metal particle, quantum dot, ceramic particle, bone particle, viral particle, etc.) surrounded by a lipid layer (e.g., lipid bilayer, lipid monolayer, etc.) comprising the lipid-PEG compounds as described herein.

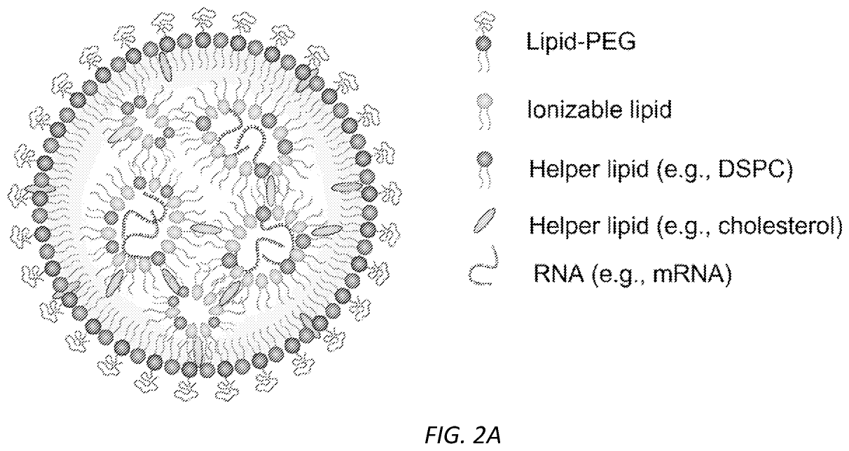

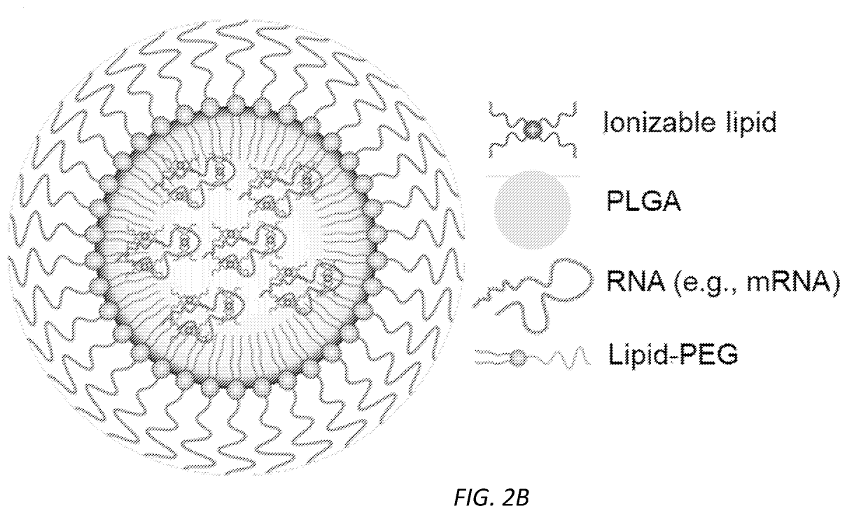

In some embodiments, the particles can comprise: i) lipid-PEG, ionizable lipid, phospholipid (e.g., DOPE or DSPC), and cholesterol (as shown in FIG. 2A), or ii) lipid-PEG, phospholipid, and cholesterol, or iii) lipid-PEG and cholesterol, or iv) lipid-PEG and phospholipid, or v) lipid-PEG only. The lipid-PEG is a Cn-X-PEG as described herein such as C14-TPA-PEG. The lipid-PEG surface layer can also contain other known lipid-PEG molecules such as DMG-PEG (C14-PEG) and DSPE-PEG. The ionizable lipid can be, e.g., DLin-MC3-DMA (MC3) lipid, G0-C8, SM-102, ALC-0315, multi-tailed ionizable phospholipids (iPhos)³¹, or others (see Chem. Rev. 2021, 121, 20, 12181-12277)¹⁰. In some embodiments, the particles can encapsulate the payloads (e.g., RNA) inside the lipid-based NPs. In some embodiments, the payloads (e.g., RNA) can be on the surface of the lipid-based NPs by either absorption or chemical conjugation.

The percent of lipid in nanoparticles can range from 0% to 99% by weight, from 10% to 99% by weight, from 25% to 99% by weight, from 50% to 99% by weight, or from 75% to 99% by weight. In some embodiments, the percent of lipid in nanoparticles can range from 0% to 75% by weight, from 0% to 50% by weight, from 0% to 25% by weight, or from 0% to 10% by weight. In some embodiments, the percent of lipid in nanoparticles can be approximately 1% by weight, approximately 2% by weight, approximately 3% by weight, approximately 4% by weight, approximately 5% by weight, approximately 10% by weight, approximately 15% by weight, approximately 20% by weight, approximately 25% by weight, or approximately 30% by weight, or any range there between having these endpoints.

In some embodiments, lipids are oils. In general, any oil known in the art can be included in nanoparticles. In some embodiments, oil may comprise one or more fatty acid groups or salts thereof. In some embodiments, a fatty acid group may comprise digestible, long chain (e.g., C8-C50), substituted or unsubstituted hydrocarbons. In some embodiments, a fatty acid group may be a C10-C20 fatty acid or salt thereof. In some embodiments, a fatty acid group may be a C15-C20 fatty acid or salt thereof. In some embodiments, a fatty acid group may be a C15-C25 fatty acid or salt thereof. In some embodiments, a fatty acid group may be unsaturated. In some embodiments, a fatty acid group may be monounsaturated. In some embodiments, a fatty acid group may be polyunsaturated. In some embodiments, a double bond of an unsaturated fatty acid group may be in the cis conformation. In some embodiments, a double bond of an unsaturated fatty acid may be in the trans conformation.

In some embodiments, a fatty acid group may be one or more of butyric, caproic, caprylic, capric, lauric, myristic, palmitic, stearic, arachidic, behenic, or lignoceric acid. In some embodiments, a fatty acid group may be one or more of palmitoleic, oleic, vaccenic, linoleic, alpha-linolenic, gamma-linoleic, arachidonic, gadoleic, arachidonic, eicosapentaenoic, docosahexaenoic, or erucic acid. In some embodiments, the oil is a liquid triglyceride.

Suitable oils for use include plant oils and butyl stearate, caprylic triglyceride, capric triglyceride, cyclomethicone, diethyl sebacate, dimethicone 360, isopropyl myristate, mineral oil, octyldodecanol, oleyl alcohol, silicone oil, and combinations thereof.

In some embodiments, a lipid is a hormone (e.g. estrogen, testosterone), steroid (e.g., cholesterol, bile acid), vitamin (e.g. vitamin E), phospholipid (e.g. phosphatidyl choline), sphingolipid (e.g. ceramides), or lipoprotein (e.g. apolipoprotein).

In certain embodiments, a lipid to be used in liposomes can be, but is not limited to, one or a plurality of the following: phosphatidylcholine, lipid A, cholesterol, dolichol, sphingosine, sphingomyelin, ceramide, glycosylceramide, cerebroside, sulfatide, phytosphingosine, phosphatidyl-ethanolamine, phosphatidylglycerol, phosphatidylinositol, phosphatidylserine, cardiolipin, phosphatidic acid, and lyso-phophatides. Naturally occurring phospholipids can include the following: phosphatidylethanolamine, phosphatidylcholine, and phosphatidylserine. Synthetic phospholipids used in the liposomes can include dioleoylphosphatidylcholine (DOPC), distearoylphosphatidylcholine (DSPC), and dioleoylphosphatidylethanolamine (DOPE).

In certain embodiments, a targeting moiety can be conjugated to the surface of a liposome.

In some embodiments, nanoparticle-stabilized liposomes are used to deliver the disclosed nucleic acid content. By allowing small charged nanoparticles (1 nm-30 nm) to adsorb on liposome surface, liposome-nanoparticle complexes have not only the merits of bare liposomes, but also tunable membrane rigidity and controllable liposome stability. When small charged nanoparticles approach the surface of liposomes carrying either opposite charge or no net charge, electrostatic or charge-dipole interaction between nanoparticles and membrane attracts the nanoparticles to stay on the membrane surface, being partially wrapped by lipid membrane. This induces local membrane bending and globule surface tension of liposomes, both of which enable tuning of membrane rigidity. Moreover, adsorbed nanoparticles form a charged shell that protects liposomes against fusion, thereby enhancing liposome stability. In certain embodiments, small nanoparticles are mixed with liposomes under gentle vortex, and the nanoparticles stick to liposome surface spontaneously. In specific embodiments, small nanoparticles can be, but are not limited to, polymeric nanoparticles, metallic nanoparticles, inorganic or organic nanoparticles, hybrids thereof, and/or combinations thereof.

2. Lipid-Polymer Particles

In some embodiments, the nanoparticles can further comprise one or more polymers associated covalently, or non-covalently with one or more lipids. In some embodiments, nanoparticles comprise one or more phospholipids.

In some embodiments, a polymeric matrix can be surrounded by a coating layer (e.g., liposome, lipid monolayer, micelle, etc.) comprising a lipid-PEG compound as described herein. In some embodiments, the lipid monolayer shell comprises an amphiphilic compound. In some embodiments, the amphiphilic compound is lecithin. In some embodiments, the lipid monolayer is stabilized.

Specific examples of amphiphilic compounds include, but are not limited to, phospholipids, such as 1,2 distearoyl-sn-glycero-3-phosphoethanolamine (DSPE), dipalmitoylphosphatidylcholine (DPPC), distearoylphosphatidylcholine (DSPC), diarachidoylphosphatidylcholine (DAPC), dibehenoylphosphatidylcholine (DBPC), ditricosanoylphosphatidylcholine (DTPC), and dilignoceroylphatidylcholine (DLPC), incorporated at a ratio of between 0.01-60 (weight lipid/w polymer), most preferably between 0.1-30 (weight lipid/w polymer). Phospholipids that may be used include, but are not limited to, phosphatidic acids, phosphatidyl cholines with both saturated and unsaturated lipids, phosphatidyl ethanolamines, phosphatidylglycerols, phosphatidylserines, phosphatidylinositols, lysophosphatidyl derivatives, cardiolipin, and β-acyl-y-alkyl phospholipids. Examples of phospholipids include, but are not limited to, phosphatidylcholines such as dioleoylphosphatidylcholine, dimyristoylphosphatidylcholine, dipentadecanoylphosphatidylcholine dilauroylphosphatidylcholine, dipalmitoylphosphatidylcholine (DPPC), distearoylphosphatidylcholine (DSPC), diarachidoylphosphatidylcholine (DAPC), dibehenoylphosphatidylcho-line (DBPC), ditricosanoylphosphatidylcholine (DTPC), dilignoceroylphatidylcholine (DLPC); and phosphatidylethanolamines such as dioleoylphosphatidylethanolamine or 1-hexadecyl-2-palmitoylglycerophos-phoethanolamine. Synthetic phospholipids with asymmetric acyl chains (e.g., with one acyl chain of 6 carbons and another acyl chain of 12 carbons) may also be used.

In some embodiments, an amphiphilic component that can be used to form an amphiphilic layer is lecithin, and, in particular, phosphatidylcholine. Lecithin is an amphiphilic lipid and, as such, forms a phospholipid bilayer having the hydrophilic (polar) heads facing their surroundings, which are oftentimes aqueous, and the hydrophobic tails facing each other. Lecithin has an advantage of being a natural lipid that is available from, e.g., soybean, and already has FDA approval for use in other delivery devices.

In certain embodiments, the amphiphilic layer of the nanoparticle, e.g., the layer of lecithin, is a monolayer, meaning the layer is not a phospholipid bilayer, but exists as a single continuous or discontinuous layer around, or within, the nanoparticle. A monolayer has the advantage of allowing the nanoparticles to be smaller in size, which makes them easier to prepare. The amphiphilic layer is “associated with” the nanoparticle, meaning it is positioned in some proximity to the polymeric matrix, such as surrounding the outside of the polymeric matrix (e.g., PLGA), or dispersed within the polymers that make up the nanoparticle.

By covering the polymeric nanoparticles with a thin film of small molecule amphiphilic compounds, the nanoparticles have merits of both polymer- and lipid-based nanoparticles, while excluding some of their limitations. The amphiphilic compounds form a tightly assembled monolayer around the polymeric core. This monolayer effectively prevents the carried agents from freely diffusing out of the nanoparticle, thereby enhancing the encapsulation yield and slowing drug release. Moreover, the amphiphilic monolayer also reduces water penetration rate into the nanoparticle, which slows hydrolysis rate of the biodegradable polymers, thereby increasing particle stability and lifetime.

In further embodiments, targeting ligands can be conjugated to the lipid-PEG compounds as described herein prior to incorporating them into the nanoparticle. Alternatively, targeting ligands can be conjugated to the polymeric component of the nanoparticles.

a. Lipid-Conjugated Polymers

In some embodiments, the nanoparticles can further comprise a polymeric matrix, wherein the polymeric matrix comprises a lipid-terminated polymer such as polyalkylene glycol and/or a polyester. In some embodiments, the nanoparticle comprises an amphiphilic lipid-terminated polymer, where a cationic and/or an amniotic lipid is conjugated to a hydrophobic polymer. In one embodiment, the polymeric matrix comprises lipid-terminated PEG.

In some embodiments, the polymeric matrix comprises lipid-terminated copolymer. In another embodiment, the polymeric matrix comprises lipid-terminated PEG and PLGA.

In one embodiment, the lipid is 1,2 distearoyl-sn-glycero-3-phosphoethanolamine (DSPE), and salts thereof. In a preferred embodiment, the polymeric matrix comprises DSPE-terminated PEG. The lipid-PEG can then, for example, be mixed with PLGA to form a nanoparticle.

3. Hydrophobic Polymers

In some embodiments, the nanoparticles can further include an hydrophobic polymers, e.g., as a core in the nanoparticle. For these hydrophobic polymers, their NPs are prepared by using the mixture of the hydrophobic polymer and Cn-X-PEG or its combination with other amphiphilic compound (which can include, but is not limited to, one or a plurality of naturally derived lipids, lipid-like materials, surfactants, or synthesized amphiphilic compounds).

Polymers and copolymers that can be used to make the nanoparticles disclosed herein include, but are not limited to, polymers including glycolic acid units, referred to herein as “PGA”, and lactic acid units, such as poly-L-Iactic acid, poly-D-Iactic acid, poly-D,L-Iactic acid, poly-L-Iactide, poly-D-Iactide, and poly-D,L-Iactide, collectively referred to herein as “PLA”, and caprolactone units, such as poly(8-caprolactone), collectively referred to herein as “PCL”; and copolymers including lactic acid and glycolic acid units, such as various forms of poly(lactic acid-co-glycolic acid) and poly(lactide-co-glycolide) characterized by the ratio of lactic acid:glycolic acid, collectively referred to herein as “PLGA”; polyacrylates, polyanhydrides, poly (ester anhydrides), poly-4-hydroxybutyrate (P4HB) combinations and derivatives thereof.

The polymer is preferably a biocompatible polymer. One simple test to determine biocompatibility is to expose a polymer to cells in vitro; biocompatible polymers are polymers that typically will not result in significant cell death at moderate concentrations, e.g., at concentrations of 50 micrograms/10⁶ cells. For instance, a biocompatible polymer may cause less than about 20% cell death when exposed to cells such as fibroblasts or epithelial cells, even if phagocytosed or otherwise uptaken by such cells.

The biocompatible polymer is preferably biodegradable, i.e., the polymer is able to degrade, chemically and/or biologically, within a physiological environment, such as within the body.

In some embodiments, the nanoparticles comprise amphiphile-polymer particles, e.g., comprising a water-insoluble polymeric core and a payload and at least one amphiphile within the core, as described in WO2016/065306, which is incorporated herein by reference in its entirety.

In preferred embodiments, the nanoparticles comprise a core of mRNA complexed with ionizable G0-Cm (or other ionizable lipids) and poly(lactic-co-glycolic acid) (PLGA) polymer, coated with a Cn-X-PEG shell or its mixture with traditional lipid-PEG shell (e.g., DSPE-PEG (1,2-distearoyl-sn-glycero-3-phosphoethanolamine-N-[methoxy{polyethylene glycol}]) or ceramide-PEG (N-palmitoyl-sphingosine-1-(succinyl{methoxy[polyethylene glycol]}) with PEG molecular weight (MW) 2000-5000)⁴⁵. G0-Cm or other ionizable lipids can be used for mRNA complexation, and PLGA, a widely clinically used biodegradable and biocompatible polymer, provides a stable NP core. Other polymers can also be included, e.g., stimuli-responsive polymers, pH Dependent Polymers, Temperature Dependent Polymers, Polymers with Dual Stimuli-Responsiveness, Polymers with Binding or Biological Responsiveness, Light-sensitive polymers, Electric field-sensitive polymers, or Hydrogel-Forming Polymers e.g., as described in WO 2018/089688, which is incorporated herein by reference in its entirety.

4. Moieties Attached to Particles

The compositions, e.g., particles, can include binding moieties or targeting moieties that specifically bind to a target cell or tissue, optionally linked to the PEG of the lipid-PEG compounds as described herein. Representative targeting moieties include, but are not limited to, antibodies and antigen binding fragments thereof, aptamers, peptides, and small molecules. The binding moiety can be conjugated to a polymer that forms the nanoparticle. Typically the binding moiety is displayed on the outer shell of the nanoparticle. The outer shell serves as a shield to prevent the nanoparticles from being recognized by a subject's immune system thereby increasing the half-life of the nanoparticles in the subject. The nanoparticles also contain a hydrophobic core. In preferred embodiments, the hydrophobic core is made of a biodegradable polymeric material. The inner core carries therapeutic payloads and releases the therapeutic payloads at a sustained rate after systemic, intraperitoneal, oral, pulmonary, or topical administration. The nanoparticles also optionally include a detectable label, for example a fluorophore or NMR contrast agent that allows visualization of nanoparticles within plaques.

The targeting moiety of the nanoparticle can be an antibody or antigen binding fragment thereof. The targeting moieties should have an affinity for a cell-surface receptor or cell-surface antigen on the target cells. The targeting moieties may result in internalization of the particle within the target cell.

The targeting moiety can specifically recognize and bind to a target molecule specific for a cell type, a tissue type, or an organ. The target molecule can be a cell surface polypeptide, lipid, or glycolipid. The target molecule can be a receptor that is selectively expressed on a specific cell surface, a tissue or an organ. Cell specific markers can be for specific types of cells including, but not limited to stem cells, skin cells, blood cells, immune cells, muscle cells, nerve cells, cancer cells, virally infected cells, and organ specific cells. The cell markers can be specific for endothelial, ectodermal, or mesenchymal cells. Representative cell specific markers include, but are not limited to cancer specific markers.

Exemplary targets include PSMA; GAH; HER2; Tf receptor; EpCAM; gC1qR (p32); Nucleolin; αvβ3/5; Collagen IV; Fibronectin; FA receptor; and Mitochondria. Exemplary methods and moieties for targeting cancer cells, including proteins, peptides, nucleic acid-based ligands, sugars, and small molecules, are described below and in Bertrand et al., Adv Drug Deliv Rev. 2014 February; 66: 2-25 (see esp. table 2 and section 3.4, “Targeting Ligands”).

a. Peptide Targeting Moieties

In a preferred embodiment, the targeting moiety is a peptide. Specifically, the plaque targeted peptide can be, but is not limited to, one or more of the following: RGD, iRGD(CRGDK/RGPD/EC), LyP-1, P3(CKGGRAKDC), or their combinations at various molar ratios. The targeting peptides can be covalently associated with the polymer and the covalent association can be mediated by a linker. The peptides target to actively growing (angiogenic) vascular endothelial cells. Those angiogenic endothelial cells frequently appear in metabolic tissues such as adipose tissues.

b. Antibody Targeting Moieties

The targeting moiety can be an antibody or an antigen-binding fragment thereof. The antibody can be any type of immunoglobulin that is known in the art. For instance, the antibody can be of any isotype, e.g., IgA, IgD, IgE, IgG, IgM, etc. The antibody can be monoclonal or polyclonal. The antibody can be a naturally-occurring antibody, e.g., an antibody isolated and/or purified from a mammal, e.g., mouse, rabbit, goat, horse, chicken, hamster, human, etc. Alternatively, the antibody can be a genetically-engineered antibody, e.g., a humanized antibody or a chimeric antibody. The antibody can be in monomeric or polymeric form. The antigen binding portion of the antibody can be any portion that has at least one antigen binding site, such as Fab, F(ab′)₂, dsFv, sFv, diabodies, and triabodies. In certain embodiments, the antibody is a single chain antibody.

c. Aptamer Targeting Moieties

Aptamers are oligonucleotide or peptide sequences with the capacity to recognize virtually any class of target molecules with high affinity and specificity. Aptamers bind to targets such as small organics, peptides, proteins, cells, and tissues. Unlike antibodies, some aptamers exhibit stereoselectivity. The aptamers can be designed to bind to specific targets expressed on cells, tissues or organs.

d. Additional Moieties

The nanoparticles can contain one or more Cn-X-PEG conjugates containing end-to-end linkages between the PEG and a moiety. The moiety can be a targeting moiety, a detectable label, or a therapeutic, prophylactic, or diagnostic agent. For example, a conjugate can be a Cn-X-PEG-phosphonate or Cn-X-PEG-galactose or Cn-X-PEG-GalNAc or Cn-X-PEG-mannose. The additional targeting elements may refer to elements that bind to or otherwise localize the nanoparticles to a specific locale. The locale may be a tissue, a particular cell type, or a subcellular compartment. The targeting element of the nanoparticle can be an antibody or antigen binding fragment thereof, an aptamer, or a small molecule (less than 500 Daltons). The additional targeting elements may have an affinity for a cell-surface receptor or cell-surface antigen on a target cell and result in internalization of the particle within the target cell.

### Cargo

In some embodiments, the nanoparticles further comprise a therapeutically or diagnostically active agent. These agents can include, for example, nucleic acids, e.g., mRNA, antisense oligonucleotides, small double-stranded RNAs (dsRNAs) such as small activating RNAs (saRNAs), small interfering RNAs (siRNAs), and microRNAs (miRNAs), or circular RNA, e.g., engineered circular RNA (oRNA), or DNA (e.g., cDNA); proteins such as antibodies, gene editing reagents (e.g., CRISPR/Cas nucleases, base editors, and so on), or other therapeutic proteins or small molecules. These nucleic acids can be chemically modified or unmodified.

In preferred embodiments, the cargo is or comprises mRNA. A mature mRNA is generally comprised of five distinct portions (see FIG. 1a of Islam et al., Biomater Sci. 2015 December; 3(12):1519-33): (i) a cap structure, (ii) a 5′ untranslated region (5′ UTR), (iii) an open reading frame (ORF), (iv) a 3′ untranslated region (3′ UTR) and (v) a poly(A) tail (a tail of 100-250 adenosine residues). Typically, the mRNA will be in vitro transcribed using methods known in the art. The mRNA will typically be modified, e.g., to extend half-life or to reduce immunogenicity. For example, the mRNA can be capped with an anti-reverse cap analog (ARCA), in which OCH₃ is used to replace or remove natural 3′ OH cap groups to avoid inappropriate cap orientation. Tetraphosphate ARCAs or phosphorothioate ARCAs can also be used (Islam et al. 2015). The mRNA is preferably enzymatically polyadenylated (addition of a poly adenine (A) tail to the 3′ end of mRNA), e.g., to comprise a poly-A tail of at least 100 or 150 As. Typically poly(A) polymerase is used; E. coli poly(A) polymerase (E-PAP) I has been optimized to add a poly(A) tail of at least 150 adenines to the 3′ terminal of in vitro transcribed mRNA. Preferably, any adenylate-uridylate rice elements (AREs) are removed or replaced with 3′ UTR of a stable mRNA species such as β-globin mRNA. Iron responsive elements (IREs) can be added in the 5′ or 3′ UTR. In some embodiments, the mRNAs include full or partial (e.g., at least 50%, 60%, 70%, 80%, or 90%) substitution of cytidine triphosphate and uridine triphosphate with naturally occurring 5-methylcytidine and pseudouridine (ψ) triphosphate. See Islam et al., 2015, and references cited therein.

### Pharmaceutical Compositions and Methods of Administration

The methods described herein include the use of pharmaceutical compositions, e.g., nanoparticles, comprising a lipid-PEG compound as described herein, wherein the composition further comprises a cargo comprising an active ingredient, e.g., therapeutically or diagnostically active agent as described herein.

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions, e.g., an immunotherapy agent as described herein.

Pharmaceutical compositions are typically formulated to be compatible with its intended route of administration. Examples of routes of administration include parenteral, e.g., intravenous, intradermal, subcutaneous, oral (e.g., inhalation), transdermal (topical), transmucosal, and rectal administration.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, NY). For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, NJ) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, and sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds can be delivered in the form of an aerosol spray from a pressured container or dispenser that contains a suitable propellant, e.g., a gas such as carbon dioxide, or a nebulizer. Such methods include those described in U.S. Pat. No. 6,468,798.

Systemic administration of a therapeutic compound as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The pharmaceutical compositions can also be prepared in the form of suppositories (e.g., with conventional suppository bases such as cocoa butter and other glycerides) or retention enemas for rectal delivery.

The pharmaceutical compositions can be included in a container, pack, or dispenser together with instructions for administration.

### Methods of Use

Further, provided herein are methods of using the compositions described herein. The methods can be for sustained delivery of a cargo as described herein, e.g., a therapeutic cargo for treatment purposes. For example, the compositions can be used for long-term sustained release of protein or mRNA cargo to provide therapeutic proteins. The methods include administration of therapeutically, prophylactically, or diagnostically effective amounts of a composition as described herein, e.g., via a suitable route of administration, such as parenteral, e.g., intravenous, intradermal, subcutaneous, direct injection (e.g., into a tumor or other target tissue), oral, nasal (e.g., inhalation), transdermal (topical), transmucosal, and rectal administration.

Table 2 provides a list of large protein- or RNA-based therapies for different diseases. The methods and compositions described herein can be used to induce durable expression of endogenous or exogenous proteins in vivo for at least 30 days after a single dose, e.g., administered by intravenous or subcutaneous injection. Consequently, this technology enables a new type of therapeutic modality for patients with chronic disease, such as hemophilia patients, methods that require much less frequent injections as compared to current cumbersome standard-of-care treatments. The long-lasting therapy can be used as both prophylaxis and demand treatment for severe hemophilia A and B patients of all ages. With subcutaneous administration, this mRNA treatment could use a less invasive administration, which will be particularly meaningful for infants and toddlers and others with difficult venous access. Moreover, it is also possible that the mRNA therapy could be used for immune tolerance induction (ITI) for patients who have developed alloantibodies “inhibitors” to exogenous factor concentrate.

For example, in addition to hemophilia A and B, the present methods and compositions can be used for other genetic disorders resulting from protein deficiency or dysfunction, as well as other indications such as cancer, infectious diseases, and neurodegenerative diseases. More specifically, present methods and compositions can be employed in the treatment of many genetic disorders that are characterized by protein deficiencies or malfunctions, such as for example thrombotic thrombocytopenic purpura, methylmalonic academia, hereditary tyrosinemia type 1, Fabry disease, acute intermittent porphyria, alpha-1 antitrypsin deficiency, glycogen storage disease type 1, cystic fibrosis, and others. One of skill in the art will understand that this technology could also be applied to other indications such as cancer, pain, infectious diseases, and neurodegenerative diseases.

TABLE 2
Protein or RNA-based protein restoration for genetic disorders

| Disease | Target gene |
|---|---|
| Hemophilia A | F8 (Factor VIII) |
| Hemophilia B | F9 (Factor IX) |
| Propionic acidemia | PCCA/PCCB |
| Methylmalonic acidemia | MMUT, MMAA, MMAB, |
| | MMADHC, or MCEE |
| Phenylketonuria | PAH |
| Glycogen Storage Disease | G6PC or SLC37A4 |
| Ornithine transcarbamylase | OTC |
| deficiency | |
| Thrombotic | ADAMTS13 |
| thrombocytopenic purpura | |
| Tyrosinemia | FAH, TAT, or HPD |
| Fabry disease | GLA |
| Acute intermittent porphyria | PBGD |
| Alpha-1 antitrypsin | AAT |
| deficiency | |
| Cystic fibrosis | CFTR |
| Crigler-Najjar syndrome | UGT1A1 |
| Arginase deficiency | ARG1 |

The compositions can also be used to deliver protein, DNA, or RNA to trigger an immune response, e.g., for use in vaccines, e.g., RNA encoding a protein or part of a protein from a pathogen (e.g., a viral, bacterial, or fungal pathogen); or RNA encoding a tumor-associated protein to trigger an anti-tumor response. Table 3 provides a list of targets for us in RNA vaccines.

TABLE 3
RNA Vaccines

| | |
|---|---|
| Infectious diseases | Flu, COVID-19, Zika, HIV, HBV, HCV, HPV, |
| | RSV, CMV, EBV, NiV |
| | hMPV, and other viral infection diseases |
| | MRSA, Cholera, Tuberculosis, Anthrax, |
| | Tetanus, and other bacterial infection diseases |
| Cancers | prostate cancer, lung cancer, breast cancer, |
| | pancreatic cancer, liver cancer, melanoma, |
| | kidney cancer, GBM, and others |
| Autoimmune diseases | rheumatoid arthritis (RA), multiple sclerosis |
| | (MS), type 1 diabetes (T1D), allergy, and others |

The compositions can be used to deliver protein, DNA, or RNA encoding proteins or peptides that alter the immune response, e.g., pro-inflammatory cytokines or costimulatory ligands or receptors to induce or enhance an anti-tumor immune response, e.g., as listed in Table 4.

TABLE 4
RNA for immuno-oncology

| | |
|---|---|
| Cytokines | IL-2, IL-6, IL-7, IL-12, IL-15, IL-21, IL-23, |
| | IL-36γ, IFNα, IFNγ, GM-CSF, and others |
| Costimulatory | CD137L/CD137, OX40L/OX40, |
| ligand/receptors | GITRL/GITR, ICOS-L/ICOS, CD70/CD27, |
| | and others |

Tumor Suppressor mRNA

The present methods include delivering mRNA encoding a tumor suppressor to a cell (e.g., a tumor cell) lacking that tumor suppressor, e.g., to treat or reduce the risk of cancer in a subject. As used herein, a tumor suppressor is a protein that acts to reduce the potential for cancer and tumor formation by modulating cell growth, by negative regulation of the cell cycle or by promoting apoptosis. Thus, loss of a tumor suppressor (e.g., through mutation or dysregulation) can lead to unregulated cell growth and tumor development. Mutations and other alterations that are associated with cancer for each of the above are known in the art.

A number of Tumor Suppressors are known in the art. See, e.g., Table 5.

TABLE 5
RNA-based tumor suppressor restoration

| | Genetic | Associated | GenBank Acc No. | |
|---|---|---|---|---|
| GENE | Alteration(s) | Cancer(s) | mRNA | Protein |
| PTEN | Point | Prostate, breast, | AF067844.1 | AAD13528.1 |
| | mutation, | glioblastoma, | | |
| | deletion | melanoma, | | |
| | | pancreatic cancer, | | |
| | | colorectal cancer, | | |
| | | leukemia | | |
| APC | Point | Adenomatous | M74088.1 | AAA03586.1 |
| | mutation, | polyposis and | | |
| | deletion | sporadic colorectal | | |
| | | tumors | | |
| ARF | Deletion | Breast carcinomas, | AF208864.1 | AAF64278.1 |
| | | colorectal | | |
| | | adenoma, | | |
| | | glioblastoma | | |
| BMPR | Point | Gastrointestinal | NM_009758.4 | NP_033888.2 |
| | mutation | cancer | | |
| BRCA1 | Point | Ductal breast | U14680.1 | AAA73985.1 |
| | mutation | cancers, | | |
| | | Epithelial ovarian | | |
| | | cancers | | |
| E-cadherin | Point | Loss of function | Z13009.1 | CAA78353.1 |
| | mutation | leads to metastasis | | |
| EXT1, 2 | Point | Hereditary multiple | S79639.1, | AAB62283.1 |
| | mutation, | exostoses, also | U62740.1 | AAB07008.1 |
| | deletion, | known as | | |
| | insertion | diaphyseal aclasis | | |
| FBXW7 | Point | Breast cancer | AF411971.1 | AAL06290.1 |
| | mutation, | | | |
| | deletion | | | |
| FH | Point | Hereditary | BC003108.1 | AAH03108.1 |
| | mutation | leiomyomatosis | | |
| | | and renal-cell | | |
| | | cancer | | |
| GPC3 | Deletions, | Lung carcinoma | L47125.1 | AAA98132.1 |
| | point | | | |
| | mutation | | | |
| HIPK2 | Point | Metastatic bladder | AF208291 | AAG41236.1 |
| | mutation | cancer | | |
| HRPT2 | Point | Hereditary | DQ366291 | |
| | mutation | hyperparathyroidis | | |
| | | m-jaw tumor | | |
| | | syndrome, | | |
| | | Malignancy in | | |
| | | sporadic | | |
| | | parathyroid tumors | | |
| INPP4B | Deletion, loss | Epithelial | U96922.1 | AAB72153.1 |
| | of | carcinomas and | | |
| | heterozygo- | some human basal- | | |
| | sity, reduced | like breast | | |
| | expression | carcinomas | | |
| LKB1 | Point | Human Lung | U63333.1 | AAB05809.1 |
| | mutation, | Cancer (especially | | |
| | deletion | NSCLC), cervical | | |
| | | carcinomas | | |
| | | Inherited cancer | | |
| | | disorder Peutz- | | |
| | | Jeghers Syndrome | | |
| MEN1 | Point | Pituitary tumors | U93236.1 | AAC51228.1 |
| | mutation | | | |
| MMR | Point | Hereditary non- | | |
| genes | mutation, | polyposis colon | | |
| | reduced | cancer | | |
| | expression | | | |
| MUTYH | Point | Lung and ovarian | U63329.1 | AAC50618.1 |
| | mutation, | tumors, and | | |
| | deletion | lymphomas | | |
| NF1 | Point | Juvenile | NM_000267.3 | NP_000258.1 |
| | mutation, | myelomonocytic | | |
| | deletion | leukemia, Watson | | |
| | | syndrome and | | |
| | | breast cancer | | |
| NF2 | Point | Meningioma | L11353.1 | AAA36212.1 |
| | mutation, | Thyroid cancer, | | |
| | deletion | mesothelioma, and | | |
| | | melanoma | | |
| p15, p16 | Point | Colorectal cancer, | AB060808.1 | BAB91133.1 |
| | mutation | leukemia | L27211.1 | AAA92554.1 |
| p53 | Point | Lung cancer, | AF307851.1 | AAG28785.1 |
| | mutation, | prostate cancer, | | |
| | deletion | liver cancer, breast | | |
| | | cancer, melanoma, | | |
| | | GBM, kidney | | |
| | | cancer, and others | | |
| p57 | Point | Beckwith- | D64137.1 | BAA11014.1 |
| | mutation | Wiedemann | | |
| | | syndrome | | |
| Ptch | Point | Cell carcinomas of | AI494442.1 | Q13635 |
| | mutation | the skin, ovarian | NM_000264.4 | NP_000255.2 |
| | | fibromas, and | | |
| | | medulloblastomas | | |
| Rb | Point | Prostate cancer | M15400.1 | AAA69807.1 |
| | mutation, | Pituitary | | |
| | deletion | melanotroph | | |
| | | tumors | | |
| RECQL4 | Point | Osteosarcoma | AB006532.1 | BAA74453.1 |
| | mutation | | | |
| SDH | Point | Paraganglioma, | U17248.1 | AAA81167.1 |
| | mutation, | renal cell | | |
| | deletion | carcinoma | | |
| Smad2/3 | Point | Breast cancer | U65019.1 | AAB17054.1 |
| | mutation, | | BC050743.1 | AAH50743.1 |
| | deletions | | | |
| Smad4 | Point | Pancreatic | U44378.1 | AAA91041.1 |
| | mutation | Gastric Carcinoma | | |
| Su(Fu) | Point | Brain tumor | Not available | Not available |
| | mutation, | | | |
| | deletion | | | |
| TGFβR | Point | Head and neck | Not available | 5E92_A |
| | mutation | cancers, cervical | | |
| | | and ovarian | | |
| | | carcinomas | | |
| TSC1/ | Point | Tuberous sclerosis | AF013168.1 | AAC51674.1″ |
| TSC2 | mutation | complex | AB014460.1 | BAA32694.1″ |
| VHL | Point | Renal carcinomas | L15409.1 | AAB64200 |
| | mutation, | | | |
| | deletion, | | | |
| | hyper- | | | |
| | methylation | | | |
| WT1 | Point | Haematological | NM_000378.4 | NP_000369.3 |
| | mutation, | malignancies | | |
| | deletion | Pediatric | | |
| | | nephroblastoma | | |
| | | Wilms tumor | | |
| XPA, | Point | Bladder cancer | D14533.1 | BAA03403.1 |
| C, D | mutation | | | |
| α-catenin | Point | Basal-like breast | HUMACA | BAA02979 |
| | mutation | cancer | | |
| RASSFIA | Hyper- | Lung, Cervical | NM_007182 | NP_009113 |
| | methylation, | Cancer | | |
| | point | | | |
| | mutation | | | |
| SDHB | Point | Kidney | KR710096 | |
| | mutation | Paragangliomas | | |
| SIN3B | Point | Prostate cancer | | AAI10822 |
| | mutation | | | |
| RGS12 | Point | Prostate cancer | AF035152 | AAC39835 |
| | mutation | | | |
| Kai1 | Deletion, | Prostate cancer | HSU20770 | CAG47051 |
| metastasis | mutation and | | | |
| suppressor | loss of | | | |
| | expression | | | |
| ING1B | Point | Prostate cancer, | AJ310392 | NP_937861 |
| | mutation | Brain tumors | | |
| Atg7 | Deletion | Prostate cancer | BC000091 | ATG7_HUMAN |
| JARID1D | Point | Prostate cancer | Not available | AAI46768 |
| | mutation | | | |
| PALB2 | Point | Breast cancer | NM_024675 | AAH44254 |
| | mutation | | | |
| 53BP1 | Point | Breast cancer | NM_024675 | AAH44254 |
| | mutation | | | |
| RAD51 | Point | Breast cancer | HSU09477 | 1GZH_D |
| | mutation | | | |
| XRCC4 | Point | Breast cancer | HUMRAD51 | CAG38796 |
| | mutation | | | |
| KEAP1 | Point | Liver cancer | AB017445 | BAB20668 |
| | mutation | | | |
| ARIAD1A | Point | Liver cancer | NM_012289 | AAH15945 |
| | mutation | | | |
| Ariad2 | Point | Liver cancer | Not available | Not available |
| | mutation | | | |
| Rps6ka3 | Point | Liver cancer | Not available | Not available |
| | mutation | | | |
| RARβ | Point | Lung cancer | BC096303 | BAC81131 |
| | mutation | | | |
| FHIT | Point | Lung cancer | NM_001290276 | BAH02279 |
| | mutation | | | |
| PTCH1 | Point | Lung cancer | HSU46922 | AAH32336 |
| | mutation | | | |
| DCC | Point | Colorectal cancer | KY652975 | AAH43542 |
| | mutation | | | |
| Bax | Point | Colorectal cancer | NM_005215 | NP_005206 |
| | mutation | | | |
| AML1 | Point | Acute myeloid | HUMBAXA | NP_620116 |
| | mutation | leukemia | | |
| CDKN2A | Point | Bladder | X90981 | BAA14022 |
| | mutation | | | |
| Cdkn1b | Point | Prostate cancer | JQ694044 | AFN61600 |
| | mutation | | | |
| NKX3.1 | Point | Prostate cancer | NM_004064 | CAG33680 |
| | mutation | | | |
| P14 | Point | Melanoma | NM_006167 | AAB38747 |
| | mutation | | | |
| CDK4 | Point | Melanoma | NM_001098783 | NP_008973 |
| | mutation | | | |
| CDK6 | Point | Melanoma | NM_000075 | CAG47043 |
| | mutation | | | |

In addition, the present methods and compositions can be used to deliver proteins, DNA, or RNA to provide therapeutic proteins; examples are listed in Table 6.

TABLE 6
RNA-based therapeutic protein secretion locally or systemically

| Application | Example target |
|---|---|
| Ischemia | VEGF-A |
| Bone regeneration | BMP-2 and VEGF-A |
| Systemic secretion of | Antibody |
| therapeutic proteins | Relaxin |
| | GLP-1 agonist |

The sequences provided herein are exemplary, as some of the above genes may have multiple transcript variants; generally speaking, the methods can include using an mRNA sequence for the variant that is predominantly expressed in a normal, non-cancerous cell of the same type as the tumor. The methods can include using a nucleotide sequence coding for an mRNA that is at least 80% identical to a reference sequence in Table 2. In some embodiments, the nucleotide sequences are at least 85%, 90%, 95%, 99% or 100% identical.

To determine the percent identity of two sequences, the sequences are aligned for optimal comparison purposes (gaps are introduced in one or both of a first and a second amino acid or nucleic acid sequence as required for optimal alignment, and non-homologous sequences can be disregarded for comparison purposes). The length of a reference sequence aligned for comparison purposes is at least 80% (in some embodiments, about 85%, 90%, 95%, or 100%) of the length of the reference sequence. The nucleotides or residues at corresponding positions are then compared. When a position in the first sequence is occupied by the same nucleotide or residue as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. For example, the percent identity between two amino acid sequences can be determined using the Needleman and Wunsch ((1970) J. Mol. Biol. 48:444-453) algorithm which has been incorporated into the GAP program in the GCG software package, using a Blossum 62 scoring matrix with a gap penalty of 12, a gap extend penalty of 4, and a frameshift gap penalty of 5.

As noted above, the delivery vehicle (e.g., nanoparticle) can be complexed with one, two or more mRNAs (e.g., a plurality of mRNAs) that encode a single tumor suppressor, or encoding multiple tumor suppressors.

In some embodiments, e.g., wherein the cancer is prostate cancer, the mRNA is PTEN. In some embodiments, the mRNA is p53. In some embodiments, the mRNA is RB. In some embodiments, the mRNAs are PTEN and p53. In some embodiments, the mRNAs are PTEN and RB. In some embodiments, the mRNAs are RB and p53. In some embodiments, the mRNAs are PTEN, p53 and RB.

In preferred embodiments, the mRNA encodes the human PTEN tumor suppressor, and in some embodiments, the cancer is breast cancer, prostate cancer, or glioblastoma. In some embodiments, the methods include determining that a subject has a cancer that is associated with loss of a tumor suppressor, and then delivering an mRNA encoding that tumor suppressor to the subject, e.g., to the tumor in the subject. Determining that a subject has a cancer that is associated with loss of a tumor suppressor can be done using any method known in the art, e.g., obtaining a sample comprising tumor cells, and detecting the presence of a mutation or loss of a tumor suppressor in the cells, e.g., by sequencing DNA of the tumor cells and detecting a mutation that is known to be associated with oncogenesis, or by detecting a decreased level or activity of the tumor suppressor protein as compared to a reference, e.g., a reference that represents a level or activity of the protein in a normal, non-cancerous cell of the same type as the tumor cell (i.e., a cell from the same kind of tissue, a non-cancerous part of the same tissues in the same individual or in an individual who doesn't have cancer).

### Hemophilia

Hemophilia is caused by a deficiency of blood clotting factor VIII (FVIII, hemophilia A) or factor IX (FIX, hemophilia B), with a global market projected to grow from $11.8b in 2019 to $17.97b in 2027. According to the Centers for Disease Control and Prevention (CDC), there are approximately 30,000-33,000 males with hemophilia living in the United States today¹. More than half of hemophilia A patients and around 40% of hemophilia B patients have severe disease. The current most-used treatment is factor concentrate replacement, which however has formidable limitations such as burdensome frequent IV infusion (twice or more per week for hemophilia A; once per 1-2 weeks for hemophilia B)^{2,3}. Therefore, new therapeutic strategies that require infrequent dosing (e.g., monthly or less) will be highly meaningful for hemophilia patients to prevent debilitating and life-threatening bleeding.

The prophylactic (preventative) use of factor concentrates has dramatically improved health outcomes for hemophilia patients; however, access to factor, burden of frequent intravenous infusions, and cost of therapy, remain significant barriers to optimal care both in the U.S and globally. A new prophylactic option for Hemophilia A patients, emicizumab, is a subcutaneously administered bispecific antibody, that seems to provide good day-to-day prophylaxis but interferes with clinical coagulation assays, thus preventing the ability to monitor/quantify hemostatic effect. Estimating how well protected an individual is with physical activities that may increase bleed risk is not possible. This provides a particular challenge in the setting of trauma or need for surgery.

Disclosed herein is are methods and compositions for long-lasting mRNA therapy of FVIII and FIX deficiency. Due to significantly shorter half-life of human factor proteins (e.g., FVIII) in mice as compared to humans⁴, the dosing frequency of the particles described herein could be further reduced in clinical treatment. this long-lasting technology could also be exploited for other bleeding disorders secondary to clotting factor deficiencies and other diseases that require restoration of protein functions.

### Immunotherapy

In some embodiments, the methods also include co-administering an immunotherapy agent to a subject who is treated with a method or composition described herein, e.g., for the treatment of a cancer. Immunotherapy agents include those therapies that target tumor-induced immune suppression; see, e.g., Stewart and Smyth, Cancer Metastasis Rev. 2011 March; 30(1):125-40.

Examples of immunotherapies include, but are not limited to, adoptive T cell therapies or cancer vaccine preparations designed to induce T lymphocytes to recognize cancer cells, as well as checkpoint inhibitors such as anti-CD137 (BMS-663513), anti-PD1 (e.g., Nivolumab, pembrolizumab/MK-3475, Pidilizumab (CT-011)), anti-PDL1 (e.g., BMS-936559, MPDL3280A), or anti-CTLA-4 (e.g., ipilumimab; see, e.g., Kruger et al., Histol Histopathol. 2007 June; 22(6):687-96; Eggermont et al., Semin Oncol. 2010 October; 37(5):455-9; Klinke D J., Mol Cancer. 2010 Sep. 15; 9:242; Alexandrescu et al., J Immunother. 2010 July-August; 33(6):570-90; Moschella et al., Ann N Y Acad Sci. 2010 April; 1194:169-78; Ganesan and Bakhshi, Natl Med J India. 2010 January-February; 23(1):21-7; Golovina and Vonderheide, Cancer J. 2010 July-August; 16(4):342-7.

Exemplary anti-PD-1 antibodies that can be used in the methods described herein include those that bind to human PD-1; an exemplary PD-1 protein sequence is provided at NCBI Accession No. NP_005009.2. Exemplary antibodies are described in U.S. Pat. Nos. 8,008,449; 9,073,994; and US20110271358, including PF-06801591, AMP-224, BGB-A317, BI 754091, JS001, MEDI0680, PDR001, REGN2810, SHR-1210, TSR-042, pembrolizumab, nivolumab, avelumab, pidilizumab, and atezolizumab.

Exemplary anti-CD40 antibodies that can be used in the methods described herein include those that bind to human CD40; exemplary CD40 protein precursor sequences are provided at NCBI Accession No. NP_001241.1, NP_690593.1, NP_001309351.1, NP_001309350.1 and NP_001289682.1. Exemplary antibodies include those described in WO2002/088186; WO2007/124299; WO2011/123489; WO2012/149356; WO2012/111762; WO2014/070934; US20130011405; US20070148163; US20040120948; US20030165499; U.S. Pat. No. 8,591,900; including dacetuzumab, lucatumumab, bleselumab, teneliximab, ADC-1013, CP-870,893, Chi Lob 7/4, HCD122, SGN-4, SEA-CD40, BMS-986004, and APX005M. In some embodiments, the anti-CD40 antibody is a CD40 agonist, and not a CD40 antagonist.

Exemplary anti-PD-L1 antibodies that can be used in the methods described herein include those that bind to human PD-L1; exemplary PD-L1 protein sequences are provided at NCBI Accession No. NP_001254635.1, NP_001300958.1, and NP_054862.1. Exemplary antibodies are described in US20170058033; WO2016/061142A1; WO2016/007235A1; WO2014/195852A1; and WO2013/079174A1, including BMS-936559 (MDX-1105), FAZ053, KN035, Atezolizumab (Tecentriq, MPDL3280A), Avelumab (Bavencio), and Durvalumab (Imfinzi, MEDI-4736).

In some embodiments, these immunotherapies may primarily target immunoregulatory cell types such as regulatory T cells (Tregs) or M2 polarized macrophages, e.g., by reducing number, altering function, or preventing tumor localization of the immunoregulatory cell types. For example, Treg-targeted therapy includes anti-GITR monoclonal antibody (TRX518), cyclophosphamide (e.g., metronomic doses), arsenic trioxide, paclitaxel, sunitinib, oxaliplatin, PLX4720, anthracycline-based chemotherapy, Daclizumab (anti-CD25); Immunotoxin eg. Ontak (denileukin diftitox); lymphoablation (e.g., chemical or radiation lymphoablation) and agents that selectively target the VEGF-VEGFR signaling axis, such as VEGF blocking antibodies (e.g., bevacizumab), or inhibitors of VEGFR tyrosine kinase activity (e.g., lenvatinib) or ATP hydrolysis (e.g., using ectonucleotidase inhibitors, e.g., ARL67156 (6-N,N-Diethyl-D-β,γ-dibromomethyleneATP trisodium salt), 8-(4-chlorophenylthio) cAMP (pCPT-cAMP) and a related cyclic nucleotide analog (8-[4-chlorophenylthio] cGMP; pCPT-cGMP) and those described in WO 2007135195, as well as mAbs against CD73 or CD39). Docetaxel also has effects on M2 macrophages. See, e.g., Zitvogel et al., Immunity 39:74-88 (2013).

In another example, M2 macrophage targeted therapy includes clodronate-liposomes (Zeisberger, et al., Br J Cancer. 95:272-281 (2006)), DNA based vaccines (Luo, et al., J Clin Invest. 116(8): 2132-2141 (2006)), and M2 macrophage targeted pro-apoptotic peptides (Cieslewicz, et al., PNAS. 110(40): 15919-15924 (2013)). Some useful immunotherapies target the metabolic processes of immunity, and include adenosine receptor antagonists and small molecule inhibitors, e.g., istradefylline (KW-6002) and SCH-58261; indoleamine 2,3-dioxygenase (IDO) inhibitors, e.g., Small molecule inhibitors (e.g., 1-methyl-tryptophan (1MT), 1-methyl-d-tryptophan (D1MT), and Toho-1) or IDO-specific siRNAs, or natural products (e.g., Brassinin or exiguamine) (see, e.g., Munn, Front Biosci (Elite Ed). 2012 Jan. 1; 4: 734-45) or monoclonal antibodies that neutralize the metabolites of IDO, e.g., mAbs against N-formyl-kynurenine.

In some embodiments, the immunotherapies may antagonize the action of cytokines and chemokines such as IL-10, TGF-beta, IL-6, CCL2 and others that are associated with immunosuppression in cancer. For example, TGF-beta neutralizing therapies include anti-TGF-beta antibodies (e.g. fresolimumab, Infliximab, Lerdelimumab, GC-1008), antisense oligodeoxynucleotides (e.g., Trabedersen), and small molecule inhibitors of TGF-beta (e.g. LY2157299), (Wojtowicz-Praga, Invest New Drugs. 21(1): 21-32 (2003)). Another example of therapies that antagonize immunosuppression cytokines can include anti-IL-6 antibodies (e.g. siltuximab) (Guo, et al., Cancer Treat Rev. 38(7):904-910 (2012). mAbs against IL-10 or its receptor can also be used, e.g., humanized versions of those described in Llorente et al., Arthritis & Rheumatism, 43(8): 1790-1800, 2000 (anti-IL-10 mAb), or Newton et al., Clin Exp Immunol. 2014 July; 177(1):261-8 (Anti-interleukin-10R1 monoclonal antibody). mAbs against CCL2 or its receptors can also be used. In some embodiments, the cytokine immunotherapy is combined with a commonly used chemotherapeutic agent (e.g., gemcitabine, docetaxel, cisplatin, tamoxifen) as described in U.S. Pat. No. 8,476,246.

In some embodiments, immunotherapies can include agents that are believed to elicit “danger” signals, e.g., “PAMPs” (pathogen-associated molecular patterns) or “DAMPs” (damage-associated molecular patterns) that stimulate an immune response against the cancer. See, e.g., Pradeu and Cooper, Front Immunol. 2012, 3:287; Escamilla-Tilch et al., Immunol Cell Biol. 2013 November-December; 91(10):601-10. In some embodiments, immunotherapies can agonize toll-like receptors (TLRs) to stimulate an immune response. For example, TLR agonists include vaccine adjuvants (e.g., 3M-052) and small molecules (e.g., Imiquimod, muramyl dipeptide, CpG, and mifamurtide (muramyl tripeptide)) as well as polysaccharide krestin and endotoxin. See, Galluzi et al., Oncoimmunol. 1(5): 699-716 (2012), Lu et al., Clin Cancer Res. Jan. 1, 2011; 17(1): 67-76, U.S. Pat. Nos. 8,795,678 and 8,790,655. In some embodiments, immunotherapies can involve administration of cytokines that elicit an anti-cancer immune response, see Lee & Margolin, Cancers. 3: 3856-3893(2011). For example, the cytokine IL-12 can be administered (Portielje, et al., Cancer Immunol Immunother. 52: 133-144 (2003)) or as gene therapy (Melero, et al., Trends Immunol. 22(3): 113-115 (2001)). In another example, interferons (IFNs), e.g., IFNgamma, can be administered as adjuvant therapy (Dunn et al., Nat Rev Immunol. 6: 836-848 (2006)).

In some embodiments, immunotherapies can antagonize cell surface receptors to enhance the anti-cancer immune response. For example, antagonistic monoclonal antibodies that boost the anti-cancer immune response can include antibodies that target CTLA-4 (ipilimumab, see Tarhini and Iqbal, Onco Targets Ther. 3:15-25 (2010) and U.S. Pat. No. 7,741,345 or Tremelimumab) or antibodies that target PD-1 (nivolumab, see Topalian, et al., NEJM. 366(26): 2443-2454 (2012) and WO2013/173223A1, pembrolizumab/MK-3475, Pidilizumab (CT-011)).

Some immunotherapies enhance T cell recruitment to the tumor site (such as Endothelin receptor-A/B (ETRA/B) blockade, e.g., with macitentan or the combination of the ETRA and ETRB antagonists BQ123 and BQ788, see Coffman et al., Cancer Biol Ther. 2013 February; 14(2):184-92), or enhance CD8 T-cell memory cell formation (e.g., using rapamycin and metformin, see, e.g., Pearce et al., Nature. 2009 Jul. 2; 460(7251):103-7; Mineharu et al., Mol Cancer Ther. 2014 Sep. 25. pii: molcanther.0400.2014; and Berezhnoy et al., Oncoimmunology. 2014 May 14; 3: e28811). Immunotherapies can also include administering one or more of: adoptive cell transfer (ACT) involving transfer of ex vivo expanded autologous or allogeneic tumor-reactive lymphocytes, e.g., dendritic cells or peptides with adjuvant; cancer vaccines such as DNA-based vaccines, cytokines (e.g., IL-2), cyclophosphamide, anti-interleukin-2R immunotoxins, and/or Prostaglandin E2 Inhibitors (e.g., using SC-50). In some embodiments, the methods include administering a composition comprising tumor-pulsed dendritic cells, e.g., as described in WO2009/114547 and references cited therein. See also Shiao et al., Genes & Dev. 2011. 25: 2559-2572.

### EXAMPLES

The invention is further described in the following examples, which do not limit the scope of the invention described in the claims.

Materials and Methods

The following materials and methods were used for the Examples below.

### Synthetic Scheme of C14-TPA-PEG (N-(methoxy polyethylene glycol)-3-(4-(phenyl(4-tetradecylphenyl)amino)phenyl) propanamide)

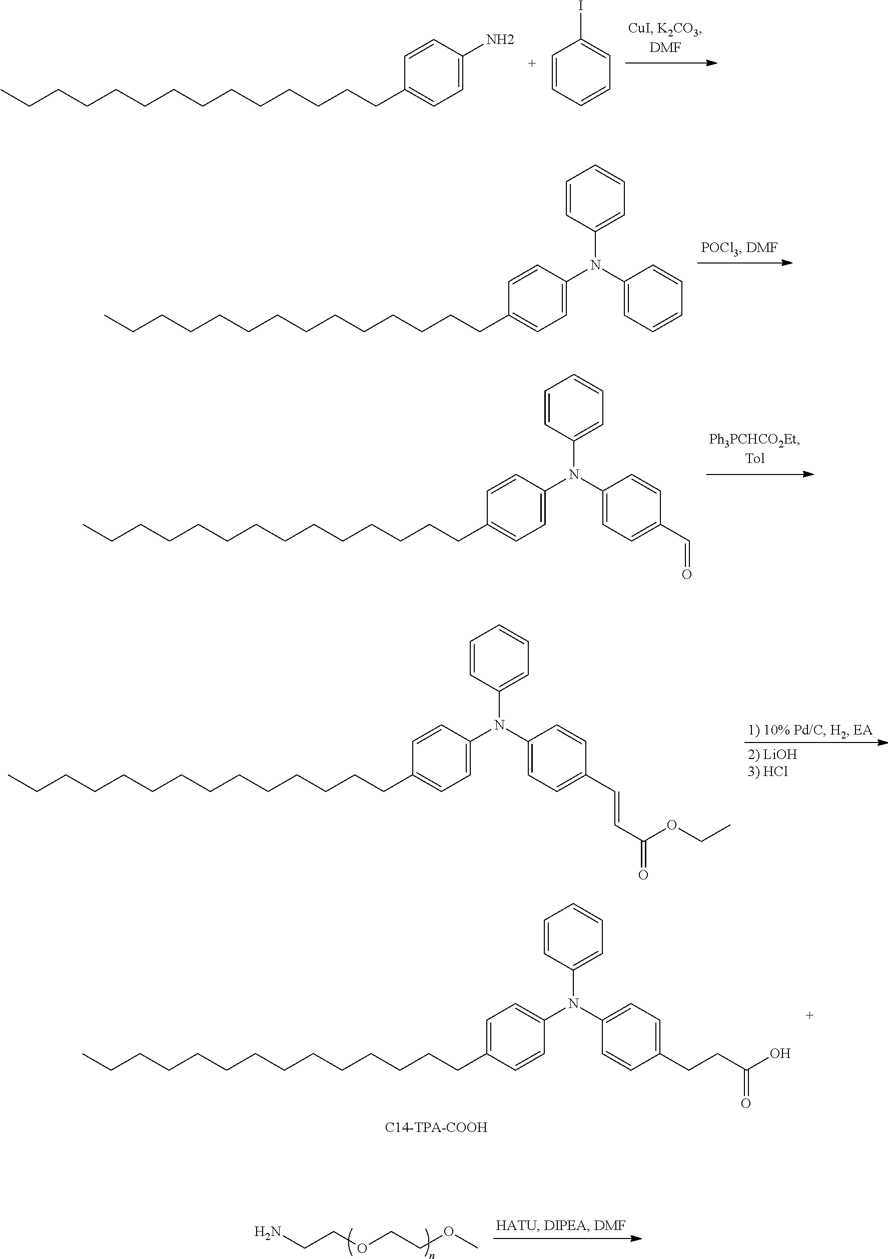

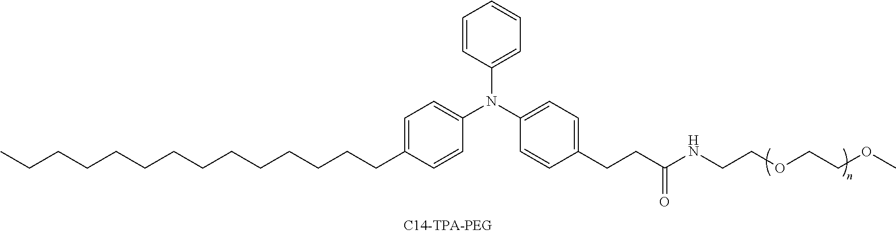

### Synthesis Steps

### Synthesis of one representative Cn-X-PEG: C14-TPA-PEG (see Scheme 1)

Step 1. Synthesis of N,N-diphenyl-4-tetradecylaniline. A mixture of 4-tetradecylaniline (1 equivalent), iodobenzene (2.5 equivalent), Cuprous iodide (CuI, 0.1 equivalent), and potassium carbonate (K₂CO₃, 3 equivalent) in N,N-dimethylformamide (DMF) was stirred under reflux in nitrogen (N₂) atmosphere. The reaction was monitored by thin layer chromatography analysis. When the reaction was completed, the reaction mixture was cooled down to room temperature. Then water was added into the reaction mixture and extracted three times of ethyl acetate. The combined organic layer was dried over anhydrous sodium sulfate (Na₂SO₄) then the solvent was removed using a rotary evaporator. The crude product was purified by flash column chromatography on silica gel to obtain the desired product N,N-diphenyl-4-tetradecylaniline.

Step 2. Synthesis of 4-(phenyl(4-tetradecylphenyl)amino)benzaldehyde. Phosphorus oxychloride (POCl₃, 10 equivalent) was added dropwise to DMF at 0° C., and the mixture was stirred for 2 h at this temperature. Then the solution of N,N-diphenyl-4-tetradecylaniline (1 equivalent) in DMF was added dropwise into the reaction mixture. The reaction mixture was warmed and stirred at room temperature. When the thin layer chromatography analysis indicated that the reaction was finished, the mixture was poured into ice water. Then the reaction mixture was extracted with ethyl acetate. The combined organic layer was dried with anhydrous Na₂SO₄ and concentrated by using a rotary evaporator. The crude product was purified by flash column chromatography on silica gel afforded the desired product 4-(phenyl(4-tetradecylphenyl)amino)benzaldehyde.

Step 3. Synthesis of ethyl (E)-3-(4-(phenyl(4-tetradecylphenyl)amino)phenyl)acrylate. Ethyl (triphenylphosphoranylidene)acetate (1.2 equivalent) was added to a solution of 4-(phenyl(4-tetradecylphenyl)amino)benzaldehyde (1 equivalent) in anhydrous toluene under a nitrogen atmosphere (N₂). The solution was stirred at room temperature and monitored by thin layer chromatography analysis. When the reaction was completed, the reaction mixture was concentrated using a rotary evaporator and the residue was purified by flash column chromatography on silica gel to give the desired product ethyl (E)-3-(4-(phenyl(4-tetradecylphenyl)amino)phenyl)acrylate.

Step 4. Synthesis of 3-(4-(phenyl(4-tetradecylphenyl)amino)phenyl)propanoic acid (C14-TPA-COOH). A mixture of ethyl (E)-3-(4-(phenyl(4-tetradecylphenyl)amino)phenyl)acrylate (1 equivalent) and 10% Pd/C (0.05 equivalent) in ethyl acetate was evacuated and back-filled with H₂ at standard atmospheric pressure (1 atm). The reaction mixture was stirred at room temperature and was monitored by thin layer chromatography analysis. When the reaction was completed, the mixture was filtered over a pad of Celite (ethyl acetate eluent) and the solvent was removed using a rotary evaporator. The crude product as the intermediate compound was used for the next step without further purification. To a solution of the intermediate compound (1 equivalent) in tetrahydrofuran (THF) was chilled to 0° C. in an ice bath. A solution of LiOH (1.1 equivalent) in H₂O was added and the reaction mixture was stirred at 0° C. for 1 hour and then warmed to room temperature. The reaction was monitored by thin layer chromatography analysis. When the reaction was completed, the reaction mixture was acidified to pH 3 with 1 M hydrochloric acid solution, extracted with ethyl acetate. The combined organic layers were dried over anhydrous Na₂SO₄ and concentrated by rotary evaporator. The crude product was purified by flash column chromatography on silica gel to give the desired product C14-TPA-COOH.

Step 5. Synthesis of C14-TPA-PEG. To a solution of compound C14-TPA-COOH (1 equivalent) and mPEG-NH₂ (1.2 equivalent, MW: 3,400 g/mol) in anhydrous DMF was added to HATU (1.5 equivalent) and DIPEA (3 equivalent) at room temperature under an argon atmosphere. The reaction mixture was stirred at room temperature. The reaction was monitored by thin layer chromatography analysis. When the reaction was completed, the crude product was dissolved in water and purified by high performance liquid chromatography (HPLC). Lyophilization of the purified material gave the desired product C14-TPA-PEG.

Preparation of mRNA lipid nanoparticles (LNPs). The mRNA lipid nanoparticles (LNPs, FIG. 2a) were prepared according to the reported ethanol dilution method with slight modification¹⁶. Briefly, mRNA was diluted in citric acid/sodium citrate buffer (10 mM, pH 3.5). The lipid mixture containing DLin-MC3-DMA (MC3), DOPE, cholesterol, and lipid-PEG (DMG-PEG2000 or Cn-X-PEGs) was prepared in ethanol. The two solutions were rapidly mixed by pipette at a 3:1 aqueous:ethanol volumetric ratio. The resulting LNPs were collected and washed three times with DNA/RNase-free pure water using an Amicon Ultra-15 centrifugal filter (molecular weight cutoff of 100 kDa, Millipore) to remove the organic solvent and free compounds. The NPs were finally dispersed in 1 mL of fresh PBS and stored at −80° C. for later use in both in vitro and in vivo studies. Parameters that could affect the encapsulation, morphology, and transfection efficiency of LNPs, such as the molar ratio among different lipid components and the weight ratio between mRNA and total lipids, were optimized.

Preparation of the polymer-lipid hybrid mRNA NPs (HNPs). A robust self-assembly technique was employed to prepare mRNA-encapsulated polymer-lipid hybrid NPs (HNPs, FIG. 2b) based on the previous report¹⁷. Briefly, G0-C8 (PAMAM generation 0 modified with alkyl chain of eight carbon length) and PLGA were dissolved separately in anhydrous DMF to form a homogeneous solution at concentrations of 2.5 mg/ml and 5 mg/ml, respectively. Lipid-PEG (e.g., DSPE-PEG or Cn-X-PEG) was dissolved in nuclease-free HyPure water (GE Healthcare Life Sciences, catalog no. SH30538) at the concentration of 1 mg/mL. All of the reagents listed above were sonicated for 5 min in a water-bath sonicator before use. Citrate buffer with pH 3.0-3.5 was first added to 80 μg of G0-C8 (in 32 μl of DMF), then 16 μg of p53 mRNA (in 16 μl of citrate buffer) was added, mixed gently (at a G0-C8/mRNA weight ratio of 5), and allowed to stay at room temperature for 15 min to ensure the sufficient electrostatic complexation. Afterwards, 250 μg of PLGA polymers (in 50 μl of DMF) was added to the mixture and gently mixed. The final mixture was added dropwise to 10 ml of DNase/RNase-free HyPure water consisting of 1 mg hybrid lipid-PEGs under uniform magnetic stirring (1000 rpm) for 30 min. An ultrafiltration device (EMD Millipore, MWCO 100 kDa) was used to remove the organic solvent and free compounds from the NP dispersion via centrifugation at 4° C. After washing 3 times with DNase/RNase-free HyPure water, the mRNA NPs were collected and finally concentrated in pH 7.4 PBS buffer. The NPs were used fresh or stored at −80° C. for further use.

Physicochemical characterization and stability of mRNA LNPs in serum condition. The hydrodynamic diameter, zeta potential and morphology of the mRNA NPs were measured to assess their physicochemical properties. Sizes and zeta potentials of the NPs were measured by dynamic light scattering (DLS, Brookhaven Instruments Corporation). Diameters are reported as the intensity mean peak average. To prepare NPs for Transmission Electron Microscopy (TEM) to characterize their morphology and shape, NPs were negatively stained with 2% uranyl acetate and then imaged with a Tecnai G2 Spirit BioTWIN microscope (FEI Company). To verify the in vitro stability of the synthesized NPs in an environment mimicking the physiological milieu, NPs were incubated in 10% serum containing PBS solution at 37° C. in triplicate for 96 hr with constant stirring at 100 rpm. At each time point, an aliquot of NP solution was withdrawn for particle size measurement using DLS and analyzed at various time intervals to evaluate any change in size distribution. The encapsulation efficiency of mRNA in NPs was analyzed with RiboGreen assay. Briefly, the mRNA NPs were firstly treated with 2% Triton X-100 to release mRNA. Then, both Triton X-100 treated and untreated mRNA NPs were incubated with RiboGreen reagent (Thermo Fisher Scientific, Cat No. R11491). The fluorescence intensity was recorded by a microplate reader to reflect the amount of total mRNA and free mRNA. The encapsulation efficiency is calculated according to the following formula: Encapsulation efficiency (%)=[(total mRNA−free mRNA)/total mRNA]×100%.

Cell culture. Pten-null prostate cancer cells (PTEN-Cap8), human liver epithelial cell THLE-3, human hepatocellular cell line Hep3B, and macrophage RAW264.7 were used for various in vitro and in vivo studies. All cells were purchased from American Type Culture Collection (ATCC). Cells were maintained in F-12K (ATCC), Dulbecco's Modified Eagle's Medium (DMEM; ATCC), Eagle's Minimum Essential Medium (EMEM; ATCC), Roswell Park Memorial Institute (RPMI) 1640 (ATCC), or Leibovitz's L-15 (ATCC) cell-culture medium, according to the culture method for each cell type per the instructions from ATCC, supplemented with high-glucose, 10% fetal bovine serum (FBS; Gibco®) and 1% penicillin/streptomycin antibiotic (Thermo-Fisher Scientific). Cell culture and all biological experiments were performed at 37° C. in 5% CO₂ conditions in a cell-culture incubator. All cells were authenticated and checked for Mycoplasma contamination before in vitro cell experiments and in vivo xenograft tumor model preparation. mRNA complexation ability and its stability in LNPs. To assess the mRNA complexation ability and its stability in LNPs and HNPs and in organic solvent (DMF), free EGFP mRNA in PBS, free EGFP mRNA in DMF or EGFP mRNA encapsulated in LNPs or HNPs were incubated at room temperature for 30 min. EGFP mRNA complexed with lipofectamine 2000 (L2K) was used as the positive control. The volumes of samples were then adjusted with loading dye (Invitrogen) and run into an E-Gel 2% agarose (Invitrogen) gel for 30 min at 50 V. The Ambion Millennium markers-Formamide (Thermo Fisher Scientific) was used as a ladder. Finally, the gel was imaged under ultraviolet and the bands were analyzed.

In vitro luciferase or EGFP expression duration study. To evaluate the transfection efficiency and duration of luciferase mRNA (Luc mRNA) or EGFP mRNA (EGFP mRNA) in THLE-3, Hep3B cells or RAW264.7 cells by different LNPs: traditional MC3 LNPs (with C14-PEG); the MC3 LNPs (with C14-TPA-PEG); and traditional LNPs with the ionizable lipid G0-C8 and C14-PEG, THLE-3 or Hep3B cells were plated in 96-well plates at a density of 5×103 cells per well. The next day, cells were treated with the various Luc mRNA LNPs or EGFP mRNA LNPs at the mRNA concentration of 0.250 μg ml⁻¹, at different time points, the cell viability were first measured by the AlamarBlue cell viability kit and the Luc transfection efficiency were measured using Steady-Glo® Luciferase Assay System (Promega) according to manufacturer's instructions. The fluorescence and luminescence were quantified using Tecan Infinite M200 Pro plate reader (Tecan). For testing the EGFP mRNA transfection and duration, at different time points, the cell viabilities were first measured by the AlamarBlue cell viability kit and the EGFP transfection were imaged using an Olympus microscope (FV1200, Olympus).

Cell growth inhibition assay with PTEN mRNA LNPs and PTEN mRNA HNPs. Cell growth inhibition was determined by Alamar Blue assay according to the manufacturer's protocol and a microplate reader (TECAN, Infinite M200 Pro). First, PTEN-Cap8 cells were plated in 96-well plates at a density of 5×10³ cells per well. The next day, cells were treated with DSPE-PLGA PTEN mRNA HNPs, C14-TPA-PEG-PLGA PTEN mRNA HNPs, MC3 PTEN mRNA LNPs or C14-TPA-PEG PTEN mRNA LNPs at different PTEN mRNA concentrations (0.250, 0.500 and 0.750 μg ml⁻¹). After 24 h of incubation, the cells were washed with 1×PBS buffer (pH 7.4) and further incubated in fresh medium for different time points. AlamarBlue cell viability was used to verify the in vitro cell growth inhibition efficacy of the PTEN mRNA NPs.

Cellular uptake. To monitor the cellular uptake of the LNPs, Cy5-Lucifease mRNA LNPs were prepared. PTEN-cap8 cells were first seeded in 35 mm confocal dishes (MatTek) at a density of 5×10⁴ cells per well and incubated at 37° C. in 5% CO₂ for 24 hours. The cells were then incubated with medium (DMEM) containing Cy5-Luc-mRNA-NPs at different mRNA concentrations (0.250, 0.500 and 0.750 μg ml⁻¹). The cells were then washed with PBS, counterstained with Hoechst 33342 (Thermofisher), and analyzed using an Olympus microscope (FV1200, Olympus).

Animals. Six-week-old C57BL/6 mice were used for pharmacokinetics (PK), in vivo luciferase expression duration study, and in vivo hEPO expression duration study. Six-week-old BALB/c white mice were used for in vivo biosafety studies. All the mice were obtained from Charles River Laboratories. All animal studies were performed under strict regulations and pathogen-free conditions in the animal facility of Brigham and Women's Hospital and in accordance with National Institutes of Health animal care guidelines. The animals had free access to sterile food pellets and water and were kept in the laboratory animal facility with temperature and relative humidity maintained at 23±2° C. and 50±20%, respectively, under a 12-h light/dark cycle. Mice were kept for at least one week to acclimatize them to the food and environment of the animal facility. The animal protocol was approved by the Institutional Animal Care and Use Committees at Harvard Medical School.

Pharmacokinetic (PK) study. Healthy C57Bl/6 mice (n=3 per group) were injected intravenously (IV) with free Cy5-EGFP-mRNA, Cy5-EGFP-mRNA DSPE-PEG LNPs, or Cy5-EGFP-mRNA C14-TPA-PEG LNPs through the tail vein at the mRNA dose of 350 μg per kg of animal weight. Blood was collected retroorbitally at different time points (5 min, 30 min, 1 hr, 2 hr, 6 hr, 12 hr, 24 hr, 36 hr, 48 hr, 96 hr and 120 hr) and the fluorescence intensity of Cy5-EGFP-mRNA was measured using a microplate reader (TECAN, Infinite M200 Pro). Pharmacokinetics was evaluated by calculating the percentage of Cy5-EGFP-mRNA in blood at various time points.

In vivo luciferase expression duration study. Healthy C57Bl/6 mice (n=3 per group) were injected IV with a single dose of the luciferase mRNA LNPs at 0.25 mg Luc-mRNA/kg body weight. At 5 hours, and day 1, 2, 3, 5, 7, 9, 11, 12, 14 and 15 after the injection of the mRNA LNPs, mice were injected intraperitoneally with 0.2 mL d-Luciferin (10 mg ml⁻¹ in PBS). The mice were anesthetized in a ventilated anesthesia chamber with 1.5% isofluorane in oxygen and imaged 8 min after the injection with an in vivo bioluminescence imaging system (Bruker Xtreme Scanner). Luminescence was quantified using the Living Image software (Bruker).

In vivo hEPO expression duration study. Healthy C57Bl/6 mice (n=3 per group) were injected IV with a single dose of the hEPO mRNA Gal-LNPs (mRNA dose: 0.25 mg/kg). At various predetermined time points (from day 0 up to day 22), ˜50 μL blood of the mice were withdrawn and EPO concentration was measured using an ELISA kit (R&D Systems).

In vivo toxicity evaluation. The in vivo toxicity of the Luc-mRNA LNPs formulated with different lipid-PEGs were evaluated by a single i.v injection of the LNPs at various mRNA dosages. 72 hours after the injection, blood was drawn, and serum was isolated at the end of the in vivo efficacy experiment. Various parameters such as ALT, AST, BUN, Albumin, Creatinine, Calcium, Phosphorus were tested to evaluate toxicity.

### Example 1. Preparation and Characterization of mRNA LNPs with Cn-X-PEG

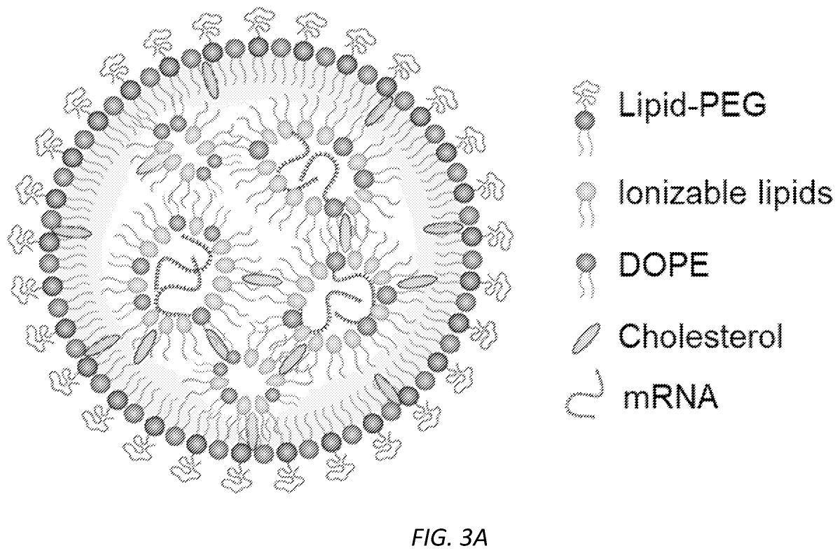

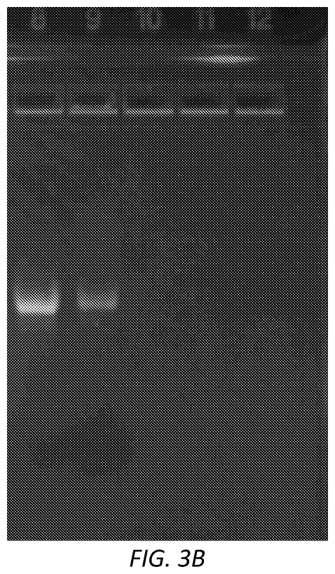

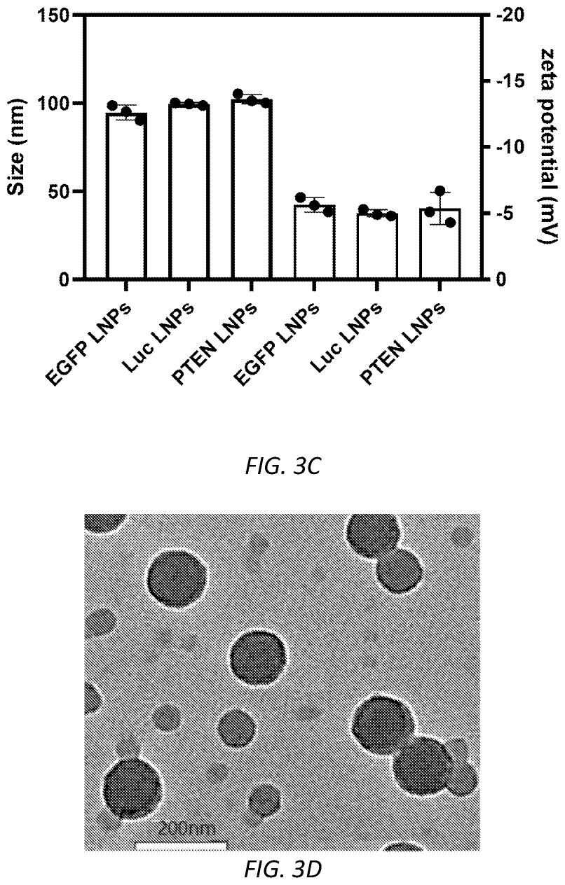

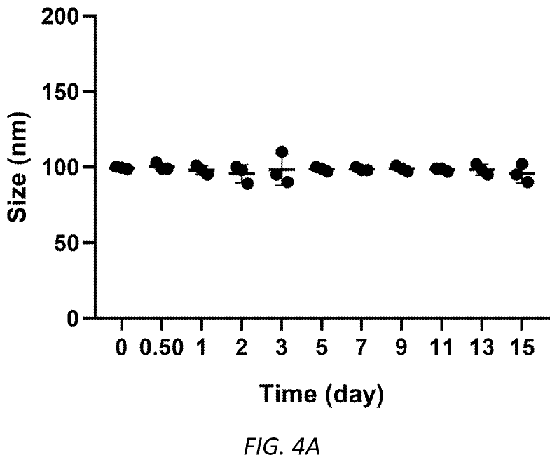

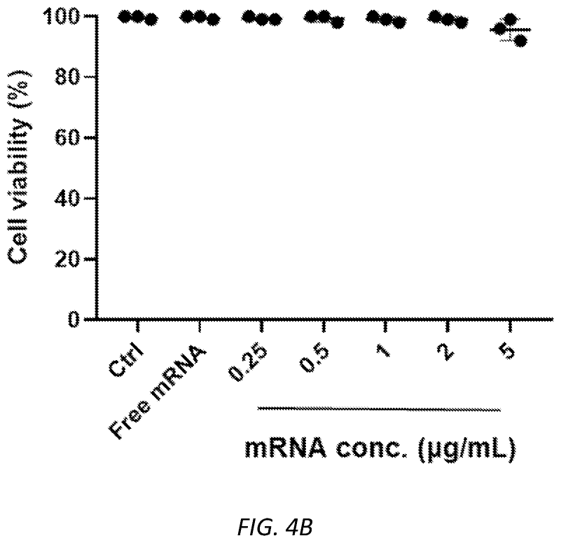

Three kinds of mRNA LNPs were prepared: the EGFP mRNA LNPs (EGFP LNPs), the Luciferase mRNA LNPs (Luc LNPs) and PTEN mRNA NPs (PTEN LNPs). LNPs were synthesized by mixing an aqueous phase containing the mRNA with an ethanol phase containing the lipids, which consists of an ionizable lipid (MC3), a phospholipid (DOPE), cholesterol, and a polyethylene glycol (PEG) containing lipid (Cn-X-PEG, FIG. 1). The ionizable lipid is positively charged at low pH to allow complexation with the negatively charged mRNA and may also help with cellular uptake and endosomal escape. The phospholipid and cholesterol are both important for the stability of the LNPs and may also help with endosomal escape. The PEGylated lipid hinders LNP aggregation, aids in vivo circulation and biodistribution, and reduces nonspecific interactions. The schematic representation of the structure of the LNPs were shown in FIG. 3a. FIG. 3b also showed that the LNP could effectively condense the EGFP mRNA with no free mRNA band shown by electrophoresis, suggesting that the mRNA was successfully encapsulated in the LNPs. The organic solvent DMF (dimethylformamide) had no effect on the integrity or stability of EGFP mRNA. The EGFP LNPs, Luc LNPs and PTEN LNPs were ˜100 nm in size and spherical, as characterized by dynamic light scattering (DLS) and transmission electron microscopy (TEM), respectively with a slightly negative surface charge around −5 mV (FIGS. 3c and 3d). Moreover, we detected no obvious changes in the size of EGFP LNPs over a period of 15 days in the presence of 10% serum, suggesting the in vitro stability of the mRNA LNPs (FIG. 4a). A cytotoxicity assay was further performed to evaluate the in vitro cytotoxicity of EGFP mRNA LNPs in PTEN-Cap8 cells at different mRNA concentrations (0.25, 0.5, 1, 2 or 5 μg/mL) (FIG. 4b). The near-100% cell viability at all tested concentrations indicated the in vitro safety of the mRNA LNPs.

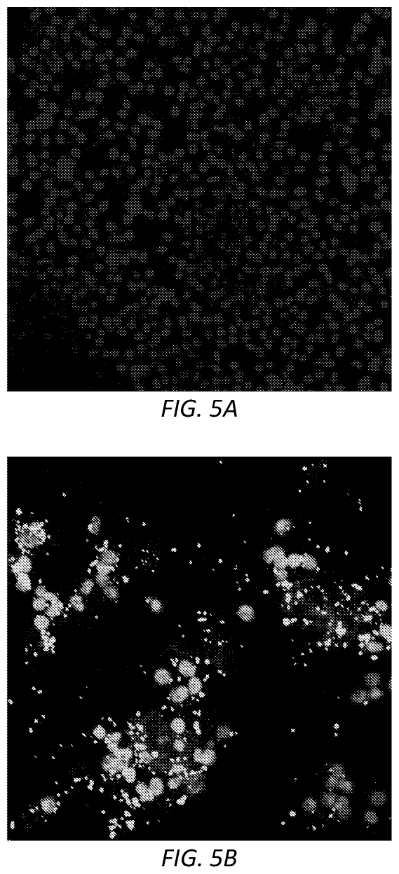

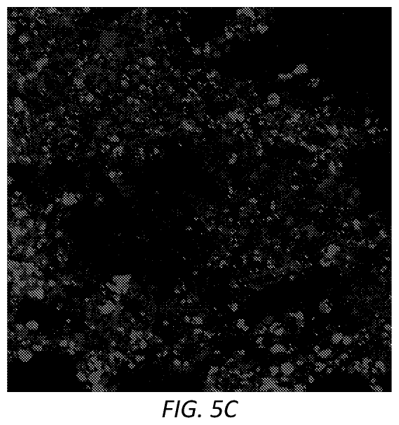

Next, intracellular uptake of the Cy5-Luc-mRNA LNPs was examined in PTEN-Cap8 cells at different mRNA concentrations by confocal fluorescence microscopy after incubating the NPs with cells for 4 hrs. The intensity of red fluorescence from Cy5-Luc mRNA in the cells increased along with the increase of mRNA concentrations (FIG. 5), suggesting the successful intracellular delivery of the mRNA LNPs.

### Example 2. Preparation and Characterization of Galactose-Modified mRNA LNPs

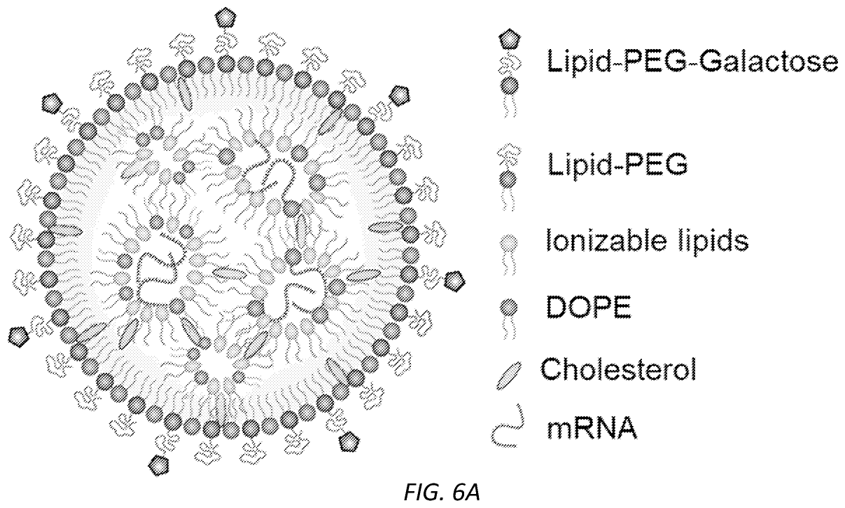

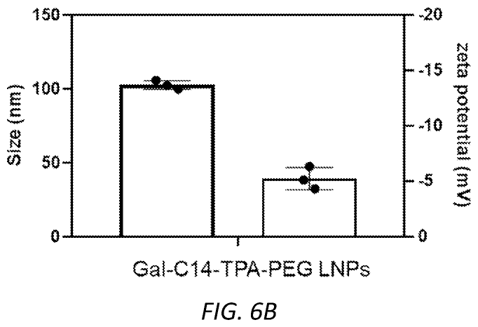

The galactose-modified mRNA LNPs (Gal-C14-TPA-PEG LNPs) were synthesized by mixing an aqueous phase containing the mRNA with an ethanol phase containing the lipids, which consists of an ionizable lipid (MC3), a phospholipid (DOPE), cholesterol, and two polyethylene glycol (PEG) containing lipid (10% Gal-C14-TPA-PEG and 90% C14-TPA-PEG) (FIG. 6a). As shown in FIG. 6b, the average particle size and zeta potential of the Gal-C14-TPA-PEG LNPs were ˜102 nm in the average particle size as measured by DLS and ˜—4.8 mV in zeta potential.

### Example 3. Preparation and Characterization of the Polymer-Lipid Hybrid mRNA NPs (HNPs) Coated with Cn-X-PEG

The HNPs (FIG. 2b) were prepared basing on a robust self-assembly strategy for formulating lipid-polymer hybrid NPs for mRNA delivery^{17,18}, which composed of the ionizable lipid-like compound G0-C8 for mRNA complexation, a biocompatible poly(lactic-co-glycolic acid) (PLGA) polymer for forming a stable NP core to carry the G0-C8/mRNA complexes, and the C14-TPA-PEG layer for stability. The HNPs showed ˜108 nm in size and a slightly negative surface charge (zeta potential of −8.3 mV).

### Example 4. Luciferase Expression in THLE-3 and Hep3B Cells by Different mRNA LNPs

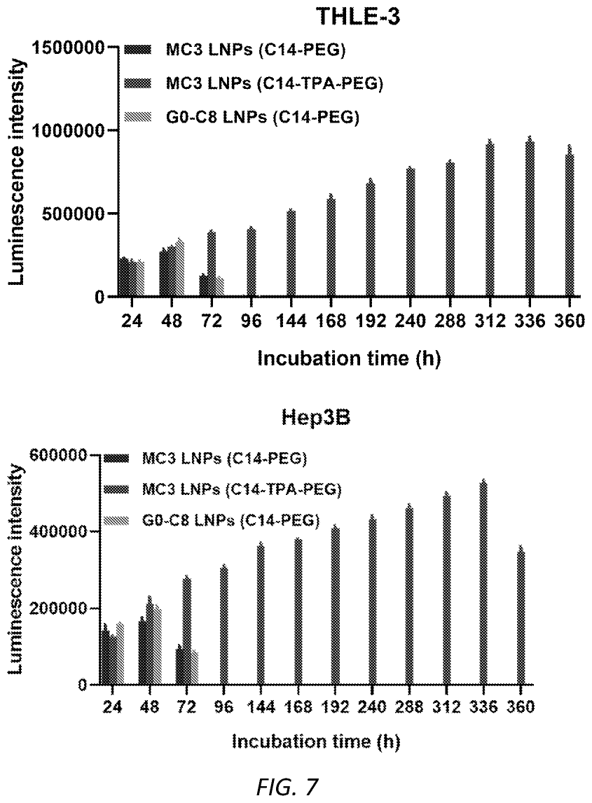

Transfection efficiency and duration of luciferase-mRNA in THLE-3 and Hep3B cells by traditional MC3 LNPs with C14-PEG (i.e., DMG-PEG); the MC3 LNPs with C14-TPA-PEG; and traditional LNPs with the ionizable lipid G0-C8 and C14-PEG were tested at the mRNA concentration of 0.25 μg/mL. As shown in FIG. 7, compared to the transient duration of the luciferase expression of the traditional MC3 LNPs with C14-PEG (˜3 days) and traditional LNPs with the ionizable lipid G0-C8 and C14-PEG (˜3 days), the LNPs formulated with C14-TPA-PEG could dramatically increase the duration of the luciferase expression for over 2 weeks.

### Example 5. GFP Expression in THLE-3 Cells Treated with the mRNA LNPs

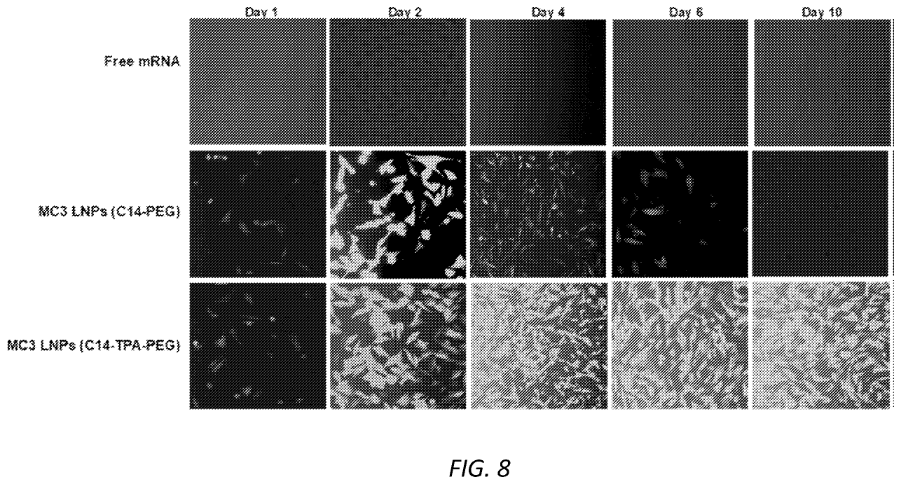

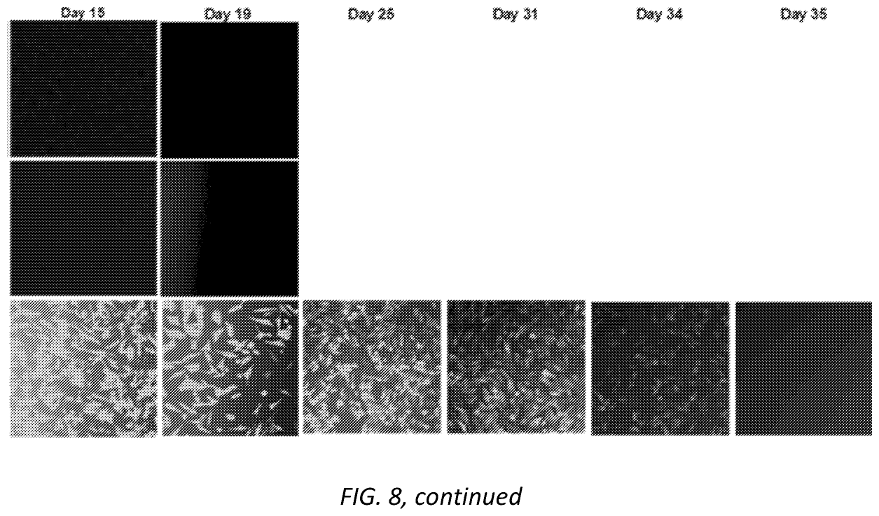

The ability of the mRNA LNPs for prolonging the duration of the protein expression was further evaluated by testing the GFP protein expression in THLE-3 cells (EGFP mRNA concentration: 0.25 μg/mL). LNPs were incubated for 1 day and then washed and replaced with fresh medium. Cells were trypsinized every 3-4 days; 6,000 cells were then re-cultured in 96-well plate. As can be seen in FIG. 8, the C14-TPA-PEG-coated LNPs can induce durable GFP expression for ˜34 days. In comparison, traditional C14-PEG-coated LNPs can only induce GFP expression for ˜6 days. Free mRNA doesn't induce protein expression. This study suggests that the mRNA LNPs can induce long-term durable protein expression in cells, which will be highly impactful for many biomedical applications and could significantly reduce the dosing frequency of mRNA therapy.

### Example 6. GFP Expression in RAW264.7 Macrophage Treated with the mRNA LNPs

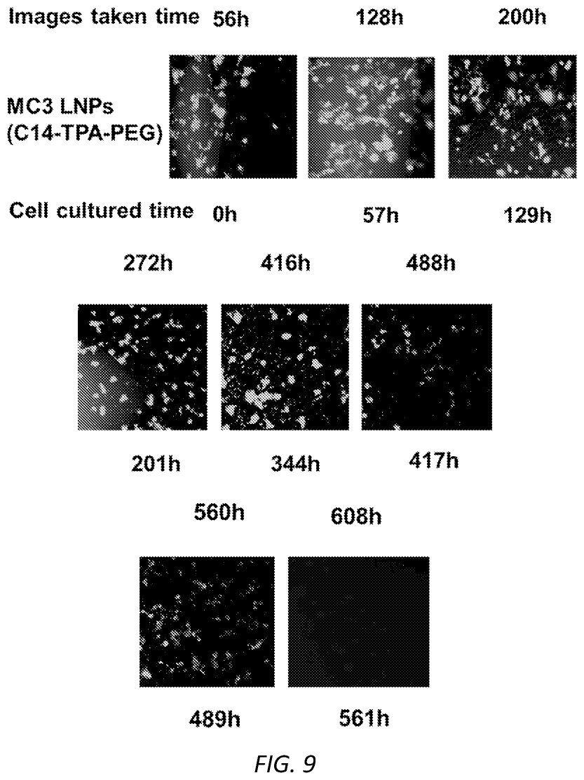

GFP expression in RAW264.7 macrophage cells after treatment with the LNPs (mRNA concentration: 0.25 μg/mL) was also tested by the microscope. As can be seen in FIG. 9, the C14-TPA-PEG-coated LNPs can induce durable GFP expression in macrophage cells for >23 days. This study indicates that the mRNA LNPs could be used for long-term engineering of therapeutic cells including immune cells, as well as for vaccine development.

### Example 7. Luciferase Expression In Vivo after a Single IV Injection with the mRNA LNPs

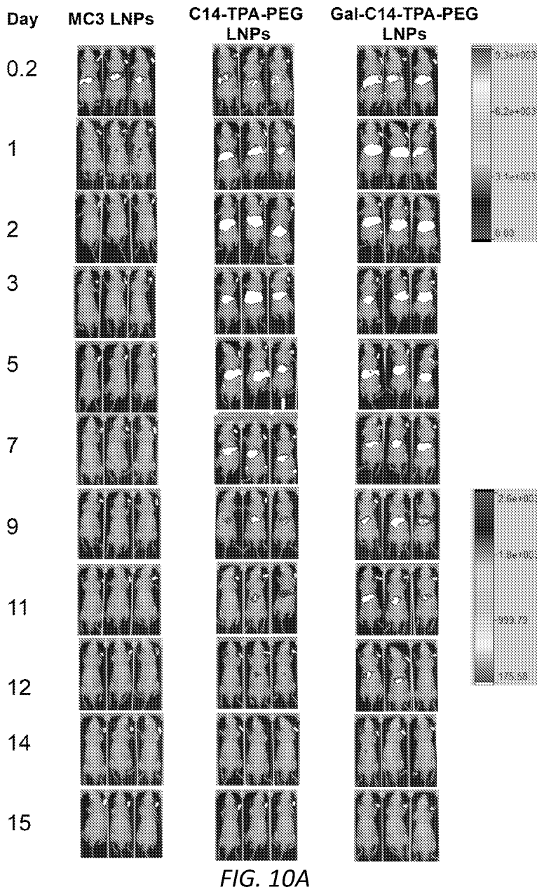

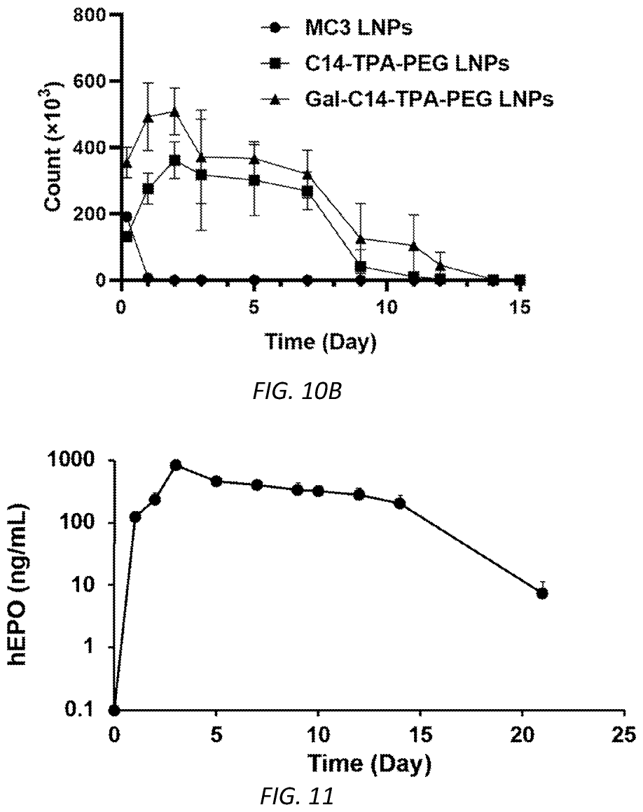

Luciferase expression in C57BL/6 mice after a single IV injection of the luciferase mRNA LNPs at different time points were performed by bioluminescence imaging. Three different kinds of LNPs were used in this study: MC3 LNPs coated with traditional DMG-PEG, C14-TPA-PEG LNPs coated with the C14-TPA-PEG, and Gal-C14-TPA-PEG LNPs coated with 10% galactose-conjugated C14-TPA-PEG and 90% C14-TPA-PEG. Both of the LNPs use the MC3 ionizable lipid for head-to-head comparison. The imaging data (FIG. 10a) and the quantification of luminescence intensity (FIG. 10b) show that the mRNA LNPs induced a strong and stable luciferase expression in vivo for 7 days and that the expression lasted ˜12-14 days. In comparison, the traditional MC3 LNPs-mediated luciferase expression in the liver only lasted for ˜2 days. This study indicates that the mRNA LNPs could be used for long-term in vivo protein replacement or generation of therapeutic proteins.

### Example 8. Human Erythropoietin (hEPO) Expression after a Single IV Injection with the mRNA LNPs

To further assess the ability of the mRNA LNPs on prolonging protein expression in vivo, hEPO expression in the blood of C57BL/6 mice were tested after a single IV treatment with the Gal-LNPs (mRNA dose: 0.25 mg/kg). The result in FIG. 11 shows a durable presence of hEPO in the blood for 14 days above 100 ng/mL and for 21 days above 7 ng/mL. This study suggests that the mRNA LNPs could be used for long-term durable protein replacement for genetic diseases (such as hemophilia, ornithine transcarbamylase deficiency, thrombotic thrombocytopenic purpura, methylmalonic academia, hereditary tyrosinemia type 1, Fabry disease, acute intermittent porphyria, alpha-1 antitrypsin deficiency, glycogen storage disease type 1, cystic fibrosis, and others) as well as other disease types (such as cancer, pain, infection diseases, neurodegenerative diseases, and others).

### Example 9. Prolonged Pharmacokinetics (PK) of the mRNA LNPs

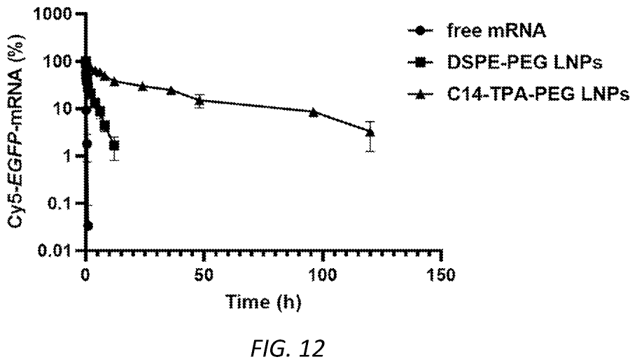

Circulation profile of free Cy5-EGFP mRNA and two different mRNA LNP formulations with DSPE-PEG (termed as DSPE-PEG LNPs) or the C14-TPA-PEG (termed as C14-TPA-PEG LNPs) were tested in normal BALB/C mice after a single IV injection of the LNPs through tail vein. As shown in FIG. 12, free mRNA was rapidly cleared, with a dramatic decrease to ˜5.6% after 15 min, and most Cy5-EGFP DSPE-PEG LNPs were eliminated after 12 hours. In contrast, LNPs formulated with the C14-TPA-PEG markedly prolonged the in vivo circulation time of the Cy5-EGFP mRNA, with ˜7% of the NPs still detectable after 120 hours (5 days). This ultra-long blood circulation may be beneficial for more effective mRNA delivery to diseased tissues such as tumor, improving therapeutic efficacy and reducing dosing frequency.

### Example 10. p⁵³ mRNA Delivery by the LNPs Reduced HCC Cell Viability In Vitro

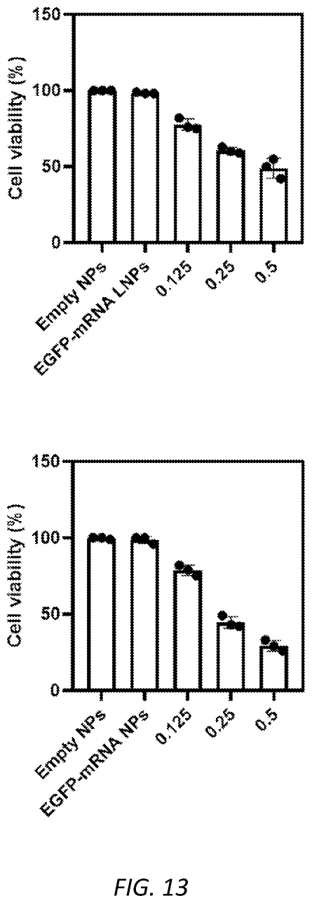

Hepatocellular carcinoma (HCC) cell viability after treatment with control EGFP-mRNA and p53-mRNA using the LNPs coated with C14-TPA-PEG were tested on two p53-null cells: murine RIL-175 cells and human Hep3B cells. The cells were incubated with empty LNPs, control EGFP-mRNA LNPs, or p53-mRNA LNPs at three different concentrations (0.125, 0.25, and 0.5 μg/mL) for 1 day, washed and further incubated with fresh medium for another day. As can be seen in FIG. 13, p53-mRNA LNPs could reduce the cell viabilities of both cell lines in a dose-dependent manner. This study indicates the possibility of using the mRNA LNPs for cancer treatment by themselves or in further combination with other therapies (such as chemotherapy, immunotherapy like immune checkpoint blockade, phototherapy, radiotherapy, and others).

### Example 11. PTEN mRNA Delivery by the Hybrid NPs or LNPs Induced Sustained Cell Growth Inhibition In Vitro

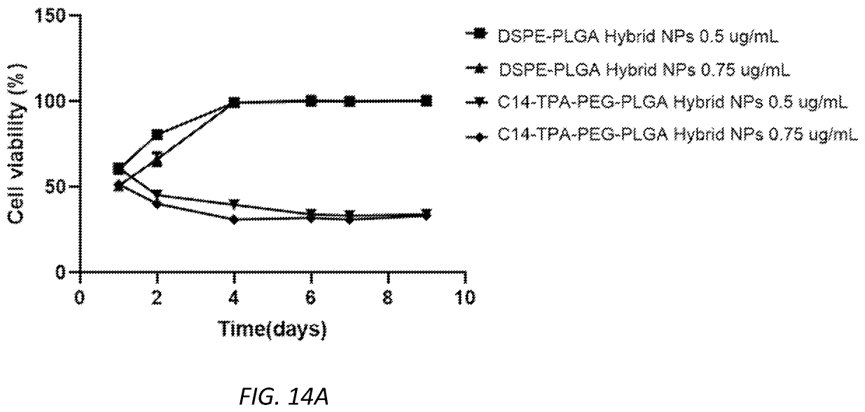

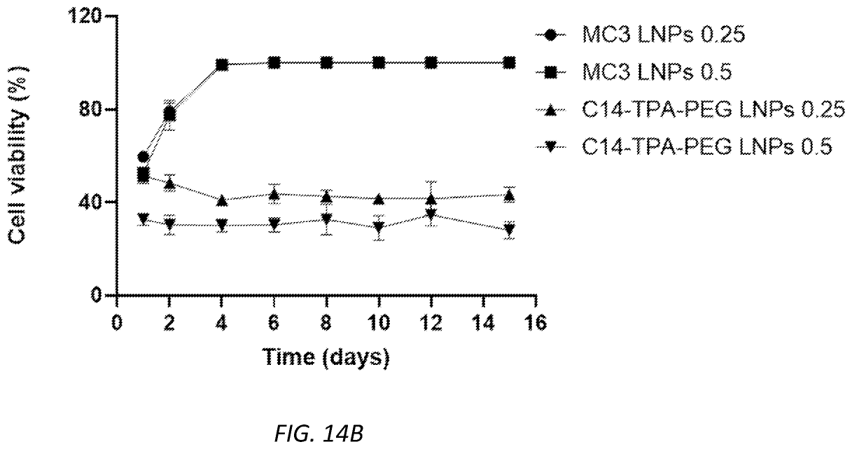

We also tested cell growth and cell viability with mRNA coding tumor suppressor PTEN. PTEN-Cap8 cells were treated with the hybrid PTEN mRNA NPs (HNPs) and the LNPs at different mRNA concentrations. As shown in FIGS. 14A-B, after one day treatment followed by cell incubation with fresh medium, both HNPs and LNPs formulated with C14-TPA-PEG showed a sustained cell growth inhibition towards PTEN-Cap8 cells while the HNPs and LNPs formulated with traditional DSPE-PEG showed transient activities.

### Example 12. Effect of Lipid-PEG Density on GFP Transfection Efficiency by EGFP-mRNA LNPs Coated with C14-TPA-PEG in THLE-3 Cells

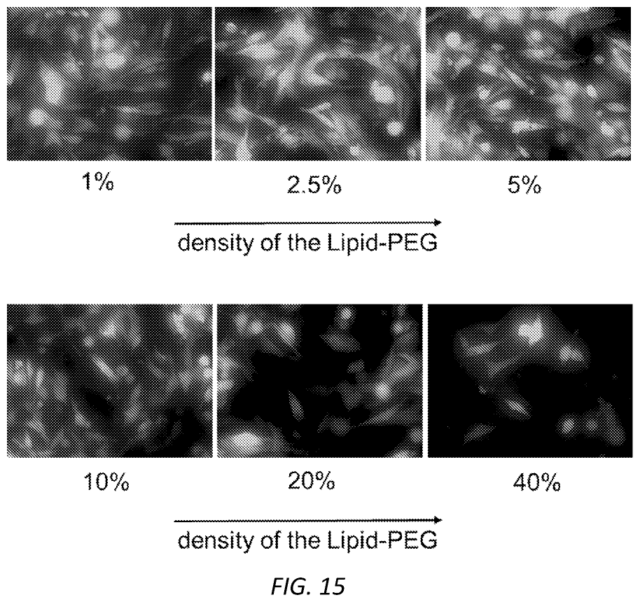

The effect of the lipid-PEG density of the EGFP-mRNA LNPs coated with C14-TPA-PEG on GFP transfection efficiency is shown in FIG. 15. There was a tendency of increasing on the EGFP transfection efficiency of the LNPs from 1% to 5%, however, after 5%, the EGFP transfection efficiency reduced as the density increases, likely due to less cellular uptake with higher PEG density.

### Example 13. Effect of Different C14-X-PEGs on Luciferase mRNA Transfection Efficiency by Luc-mRNA LNPs in THLE-3 Cells

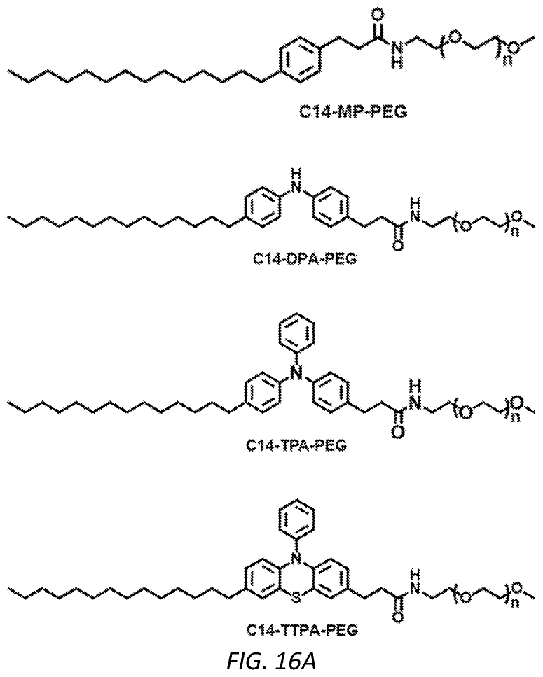

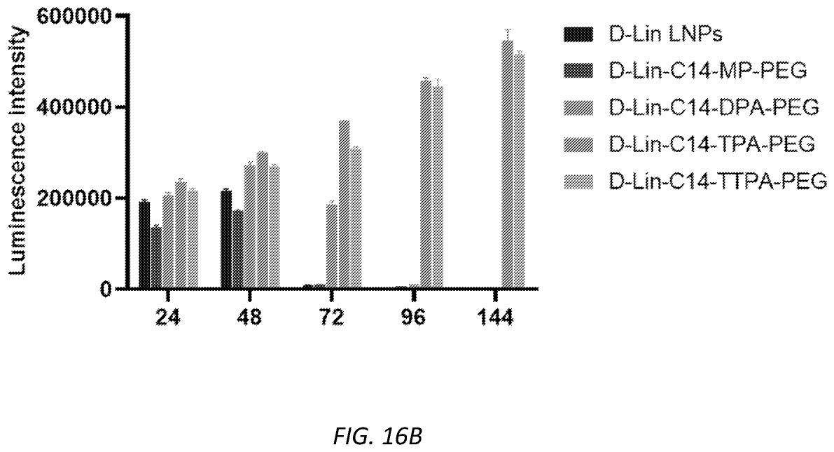

To investigate the effect of the X groups in C14-X-PEGs on the mRNA transfection efficiency, four different kinds of C14-X-PEG were synthesized and their transfection efficiency on Luciferase mRNA were compared. As shown in FIGS. 16A-B, C14-X-PEGs with more benzyl groups tended to lead stronger and longer luciferase expression.

### Example 14. Effect of Different Cn-TPA-PEGs on GFP Protein Expression Duration in TILE-3 Cells

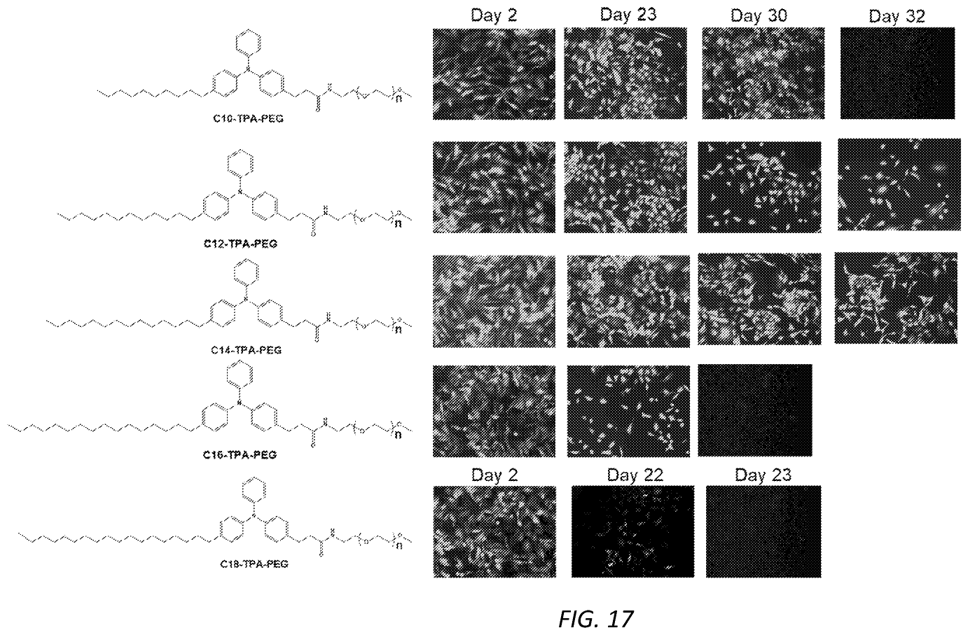

To investigate whether the length of alkyl chain plays a role in protein expression duration, a series of Cn-TPA-PEGs were synthesized and the GFP protein expression duration by EGFP-mRNA LNPs were tested in THLE-3 cells. The results showed in FIG. 17 suggested that the hydrocarbon chain length does affect the durability of protein expression. All LNPs formulated with these Cn-TPA-PEG showed durable long-term EGFP expression, with C10, C12, and C14 better than C16 and C18.

### Example 15. In Vivo Toxicity of the mRNA LNPs: Hematologic Examination

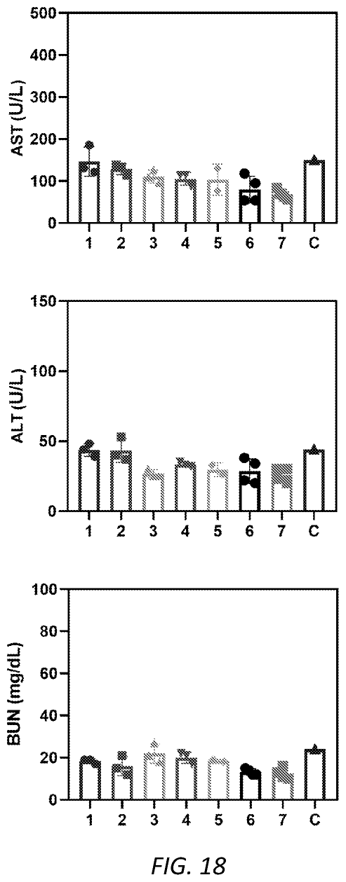

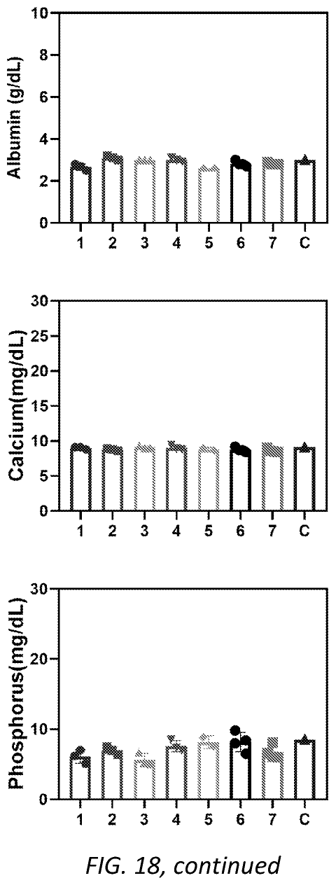

To evaluate the potential in vivo side effects of mRNA NPs, hematological analysis was performed by checking parameters including aspartate aminotransferase (AST) & alanine aminotransferase (ALT) to assess liver function, creatinine and blood urea nitrogen (BUN) to evaluate kidney activity along with other parameters including calcium and phosphorus using appropriate assay kits. We found no obvious changes in these parameters in serum from mice after treatment with the mRNA LNPs formulated with Cn-TPA-PEGs, further indicating negligible side effects (FIG. 18).

### Example 16. Preparation and Characterization of C14-TPA-PEG/EGFP mRNA Mixture

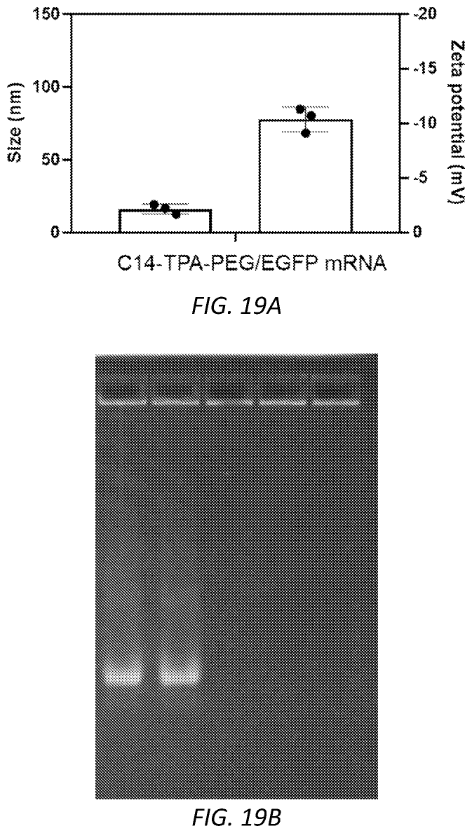

To investigate whether a simple mixture of C14-TPA-PEG with EGFP mRNA is also able to deliver mRNA and prolong the duration of the protein expression, C14-TPA-PEG/EGFP mRNA mixtures were prepared and characterized. As shown in FIGS. 19A-B, the mixtures showed ˜12 nm in size and ˜−10 mV of the zeta potential.

### Example 17. Durable EGFP Protein Expression in TILE-3 Cells by EGFP-mRNA/C14-TPA-PEG Mixture

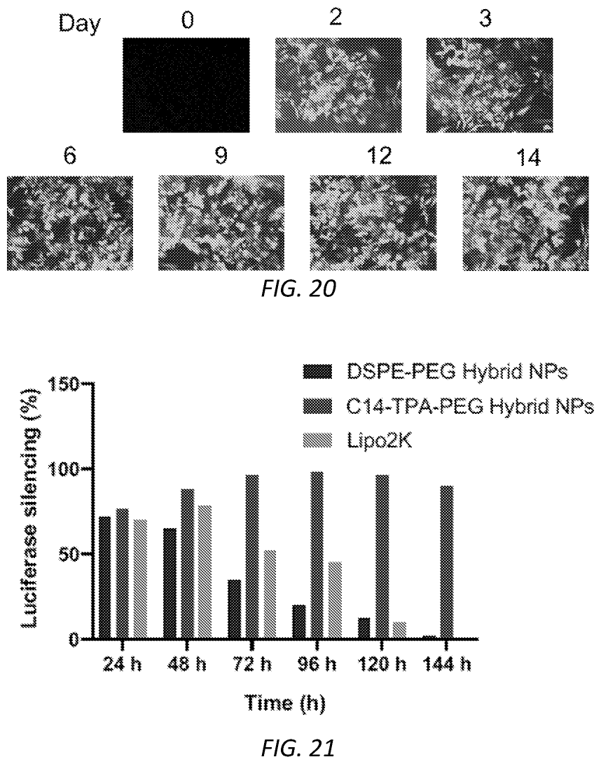

As shown in FIG. 20, C14-TPA-PEG could effectively deliver EGFP mRNA and could effectively induce GFP expression for more than 14 days. This result suggested that C14-TPA-PEG could stabilize mRNA, contributing to the long-durable mRNA activity, which will be highly impactful for many biomedical applications and could significantly reduce/eliminate the use of cationic lipids for RNA delivery.

### Example 18. Luciferase Silencing in Luc-HeLa Cells by siRNA Hybrid NPs Coated with C14-TPA-PEG

To investigate whether the lipid-PEGs will also work for other RNAs, we tested siRNA delivery and the silencing effect/durability. siLuc hybrid NPs formulated with DSPE-PEG and the lipid-PEG (C14-TPA-PEG) were prepared and their silencing effect were compared. As shown in FIG. 21, DSPE-PEG HNPs only showed effective silencing effect in 48 hours while C14-TPA-PEG HNPs showed durable effective silencing effect for at least 144 hours (6 days). This result suggests that the lipid-PEG could also be applicable to other types of RNA delivery with durable activity.

### REFERENCES

1 Sidonio, R. F., Jr. et al. Discussing investigational AAV gene therapy with hemophilia patients: A guide. Blood Rev, 100759, doi:10.1016/j.blre.2020.100759 (2020).
2 Croteau, S. E., Wang, M. & Wheeler, A. P. 2021 clinical trials update: Innovations in hemophilia therapy. Am J Hematol 96, 128-144, doi: 10. 1002/ajh.26018 (2021).
3 Van Tendeloo, V. F., Ponsaerts, P. & Berneman, Z. N. mRNA-based gene transfer as a tool for gene and cell therapy. Curr Opin Mol Ther 9, 423-431 (2007).
4 Xiong, Q., Lee, G. Y., Ding, J., Li, W. & Shi, J. Biomedical applications of mRNA nanomedicine. Nano Res 11, 5281-5309, doi:10.1007/s12274-018-2146-1 (2018).
5 Berraondo, P., Martini, P. G. V., Avila, M. A. & Fontanellas, A. Messenger RNA therapy for rare genetic metabolic diseases. Gut 68, 1323-1330, doi:10.1136/gutjnl-2019-318269 (2019).
6 Kowalski, P. S., Rudra, A., Miao, L. & Anderson, D. G. Delivering the Messenger: Advances in Technologies for Therapeutic mRNA Delivery. Mol Ther 27, 710-728, doi:10.1016/j.ymthe.2019.02.012 (2019).
7 Yin, H. et al. Non-viral vectors for gene-based therapy. Nat Rev Genet 15, 541-555, doi:10.1038/nrg3763 (2014).
8 Ferizi, M. et al. Stability analysis of chemically modified mRNA using micropattern-based single-cell arrays. Lab on a chip 15, 3561-3571, doi:10.1039/c51c00749f (2015).
9 Pardi, N. et al. Expression kinetics of nucleoside-modified mRNA delivered in lipid nanoparticles to mice by various routes. Journal of controlled release: official journal of the Controlled Release Society 217, 345-351, doi:10.1016/j.jconrel.2015.08.007 (2015).
10 Zhang, Y., Sun, C., Wang, C., Jankovic, K. E. & Dong, Y. Lipids and Lipid Derivatives for RNA Delivery. Chem Rev 121, 12181-12277, doi:10.1021/acs.chemrev.1c00244 (2021).
11 Chen, C. Y. et al. Treatment of Hemophilia A Using Factor VIII Messenger RNA Lipid Nanoparticles. Mol Ther Nucleic Acids 20, 534-544, doi:10.1016/j.omtn.2020.03.015 (2020).
12 DeRosa, F. et al. Therapeutic efficacy in a hemophilia B model using a biosynthetic mRNA liver depot system. Gene Ther 23, 699-707, doi:10.1038/gt.2016.46 (2016).
13 Akinc, A. et al. The Onpattro story and the clinical translation of nanomedicines containing nucleic acid-based drugs. Nature nanotechnology 14, 1084-1087, doi:10.1038/s41565-019-0591-y (2019).
14 Chaudhary, N., Weissman, D. & Whitehead, K. A. mRNA vaccines for infectious diseases: principles, delivery and clinical translation. Nature reviews 20, 817-838, doi:10.1038/s41573-021-00283-5 (2021).
15 Hewitt, S. L. et al. Durable anticancer immunity from intratumoral administration of IL-23, IL-36gamma, and OX40L mRNAs. Sci Transl Med 11, doi:10.1126/scitranslmed.aat9143 (2019).
16 Sabnis, S. et al. A Novel Amino Lipid Series for mRNA Delivery: Improved Endosomal Escape and Sustained Pharmacology and Safety in Non-human Primates. Mol Ther 26, 1509-1519, doi:10.1016/j.ymthe.2018.03.010 (2018).
17 Nabhan, J. F. et al. Intrathecal delivery of frataxin mRNA encapsulated in lipid nanoparticles to dorsal root ganglia as a potential therapeutic for Friedreich's ataxia. Sci Rep 6, 20019, doi:10.1038/srep20019 (2016).
18 Cheng, Q. et al. Dendrimer-Based Lipid Nanoparticles Deliver Therapeutic FAH mRNA to Normalize Liver Function and Extend Survival in a Mouse Model of Hepatorenal Tyrosinemia Type I. Adv Mater 30, e1805308, doi:10.1002/adma.201805308 (2018).
19 Liu-Chen, S., Connolly, B., Cheng, L., Subramanian, R. R. & Han, Z. mRNA treatment produces sustained expression of enzymatically active human ADAMTS13 in mice. Sci Rep 8, 7859, doi:10.1038/s41598-018-26298-4 (2018).
20 Asrani, K. H., Cheng, L., Cheng, C. J. & Subramanian, R. R. Arginase I mRNA therapy—a novel approach to rescue arginase 1 enzyme deficiency. RNA Biol 15, 914-922, doi:10.1080/15476286.2018.1475178 (2018).
21 Truong, B. et al. Lipid nanoparticle-targeted mRNA therapy as a treatment for the inherited metabolic liver disorder arginase deficiency. Proc Natl Acad Sci USA 116, 21150-21159, doi:10.1073/pnas.1906182116 (2019).
22 Ramaswamy, S. et al. Systemic delivery of factor IX messenger RNA for protein replacement therapy. Proc Natl Acad Sci USA 114, E1941-E1950, doi:10.1073/pnas.1619653114 (2017).
23 Cheng, Q. et al. Selective organ targeting (SORT) nanoparticles for tissue-specific mRNA delivery and CRISPR-Cas gene editing. Nature nanotechnology 15, 313-320, doi:10.1038/s41565-020-0669-6 (2020).
24 Russick, J. et al. Correction of bleeding in experimental severe hemophilia A by systemic delivery of factor VIII-encoding mRNA. Haematologica 105, 1129-1137, doi:10.3324/haematol.2018.210583 (2020).
25 Patel, S., Ryals, R. C., Weller, K. K., Pennesi, M. E. & Sahay, G. Lipid nanoparticles for delivery of messenger RNA to the back of the eye. J Control Release 303, 91-100, doi:10.1016/j.jconrel.2019.04.015 (2019).
26 Schumann, C. et al. Increasing lean muscle mass in mice via nanoparticle-mediated hepatic delivery of follistatin mRNA. Theranostics 8, 5276-5288, doi:10.7150/thno.27847 (2018).
27 Haabeth, O. A. W. et al. mRNA vaccination with charge-altering releasable transporters elicits human T cell responses and cures established tumors in mice. Proc Natl Acad Sci USA 115, E9153-E9161, doi:10.1073/pnas.1810002115 (2018).
28 Poliskey, J. A. et al. Metabolically stabilized double-stranded mRNA polyplexes. Gene Ther 25, 473-484, doi:10.1038/s41434-018-0038-3 (2018).
29 Prieve, M. G. et al. Targeted mRNA Therapy for Ornithine Transcarbamylase Deficiency. Mol Ther 26, 801-813, doi:10.1016/j.ymthe.2017.12.024 (2018).
30 Kim, J., Jozic, A. & Sahay, G. Naturally Derived Membrane Lipids Impact Nanoparticle-Based Messenger RNA Delivery. Cell Mol Bioeng Epub ahead of print, doi:doi: 10.1007/s12195-020-00619-y (2020).
31 Liu, S. et al. Membrane-destabilizing ionizable phospholipids for organ-selective mRNA delivery and CRISPR-Cas gene editing. Nature materials 20, 701-710, doi:10.1038/s41563-020-00886-0 (2021).

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A compound of Formula (I): 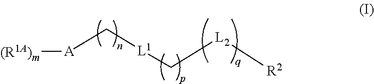
wherein:
A is selected from C₆₋₁₀ aryl and 5- to 10-membered heteroaryl, wherein the C₆₋₁₀ aryl or 5- to 10-membered heteroaryl is optionally substituted with one or more substituents independently selected from the group consisting of C₁₋₁₅ alkyl, C₂₋₁₅ alkenyl, C₂₋₁₅ alkynyl, C₃₋₁₀ cycloalkyl, C₆₋₁₀ aryl, 5- to 10-membered heteroaryl, 4- to 10-membered heterocycloalkyl, halo, —CN, —OR^{O}, —N(R^{N})₂, —(C═O)R^{N}, —(C═O)OR^{O}, —(C═O)N(R^{N})₂, and —NR^{N}(C═O)R⁸;
each R^{1A} is selected from C₁₋₁₀₀ alkyl, C₁₋₁₀₀ alkenyl, and C₁₋₁₀₀ alkynyl, and C₁₋₁₀₀ haloalkyl, wherein the C₁₃₋₁₀₀ alkyl, C₁₃₋₁₀₀ alkenyl, and C₁₃₋₁₀₀ alkynyl forming R¹ is optionally substituted with one or more substituents independently selected from the group consisting of halo, —CN, —OR^{O}, —N(R^{N})₂, —(C═O)R^{N}, —(C═O)OR^{O}, —(C═O)N(R^{N})₂, —NR^{N}(C═O)R⁸, and —O(C═O)R⁸;
L¹ is selected from bond, —N(R^{N})—, —O—, —(C═O)—, —(C═O)O—, —(C═O)N(R^{N})—, —NR^{N}(C═O)—, and —O(C═O)—;
L² is selected from: 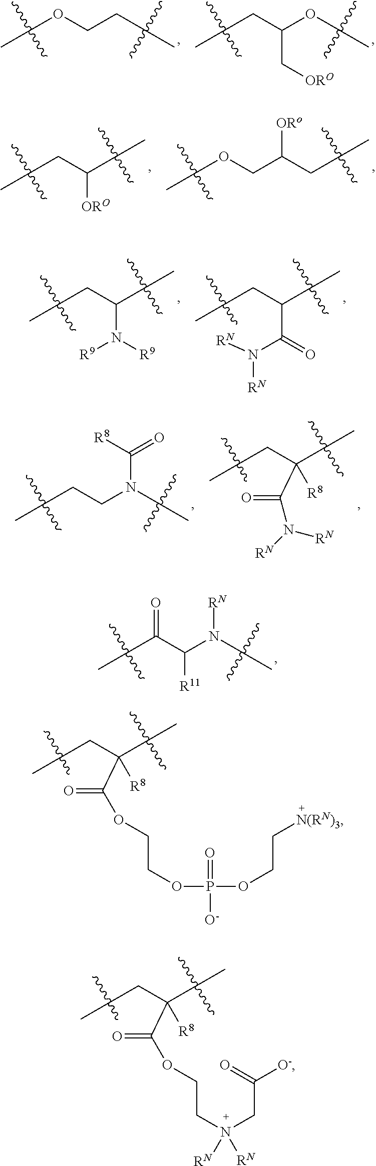
heparin, dextran, and chitosan;
R² is selected from H, C₁₋₁₅ alkyl, C₂₋₁₅ alkenyl, C₂₋₁₅ alkynyl, —OR^{O}, —(C═O)OR^{O}, —N(R^{N})₂, —N₃, 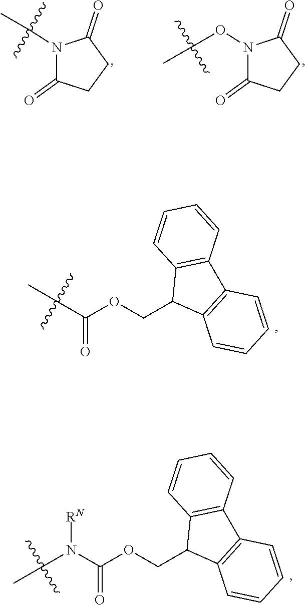
and targeting ligand;
each R⁸ is independently selected from H, C₁₋₁₅ alkyl, C₂₋₁₅ alkenyl, and C₂₋₁₅ alkynyl;
each R⁹ is independently selected from H, C₁₋₁₅ alkyl, C₂₋₁₅ alkenyl, and C₂₋₁₅ alkynyl;
or two R⁹, together with the N atom to which they are attached, come together to form 4- to 10-membered heterocycloalkyl optionally substituted with one or more oxo;
each R¹¹ is independently selected from C₁₋₁₅ alkyl, C₂₋₁₅ alkenyl, and C₂₋₁₅ alkynyl, optionally substituted with one or more R¹²;
each R¹² is independently selected from C₃₋₁₀ cycloalkyl, C₆₋₁₀ aryl, 5- to 10-membered heteroaryl, 4- to 10-membered heterocycloalkyl, halo, —CN, —OR^{O}, —N(R^{N})₂, —(C═O)R^{N}, —(C═O)OR^{O}, —(C═O)N(R^{N})₂, —NR^{N}(C═O)R⁸, —NR^{N}(C═NR^{N})R^{N}, —O(C═O)R⁸, and —SR⁸, wherein the C₃₋₁₀ cycloalkyl, C₆₋₁₀ aryl, 5- to 10-membered heteroaryl, 4- to 10-membered heterocycloalkyl is optionally substituted with one or more C₁₋₁₅ alkyl, C₂₋₁₅ alkenyl, and C₂₋₁₅ alkynyl, halo, —CN, —OR^{O}, and —N(R^{N})₂;
each R^{N} is independently selected from H, C₁₋₁₅ alkyl, C₂₋₁₅ alkenyl, and C₂₋₁₅ alkynyl, wherein the C₁₋₁₅ alkyl, C₂₋₁₅ alkenyl, and C₂₋₁₅ alkynyl forming R^{N} is optionally substituted with one or more substituents independently selected from the group consisting of halo, —CN, and —OR^{O};
each R^{O} is independently selected from H, C₁₋₁₅ alkyl, C₂₋₁₅ alkenyl, and C₂₋₁₅ alkynyl, wherein the C₁₋₁₅ alkyl, C₂₋₁₅ alkenyl, and C₂₋₁₅ alkynyl forming R^{O} is optionally substituted with one or more substituents independently selected from the group consisting of halo and —CN;
m is an integer selected from 1, 2, 3, 4, and 5;
n and p are each an integer independently selected from 0, 1, 2, 3, and 4; and
q is an integer selected from 1 to 2500;
provided that when A is phenyl, then each R^{1A} is selected from C₁₃₋₁₀₀ alkyl, C₁₋₁₀₀ alkenyl, C₁₋₁₀₀ alkynyl, and C₁₋₁₀₀ haloalkyl, wherein the C₁₃₋₁₀₀ alkyl, C₁₋₁₀₀ alkenyl, and C₁₋₁₀₀ alkynyl forming R¹ is optionally substituted with one or more substituents independently selected from the group consisting of halo, —CN, —OR^{O}, —N(R^{N})₂, —(C═O)R^{N}, —(C═O)OR^{O}, —(C═O)N(R^{N})₂, —NR^{N}(C═O)R⁸, and —O(C═O)R⁸.

2. The compound of claim 1, wherein the compound is selected from: 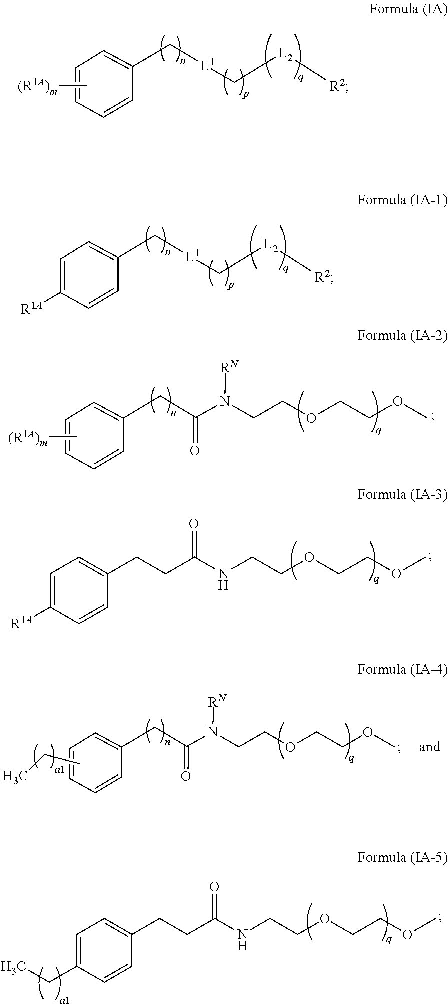
wherein a1 is an integer selected from 12 to 100.

3. The compound of claim 1, wherein the compound is of Formula (I-1):

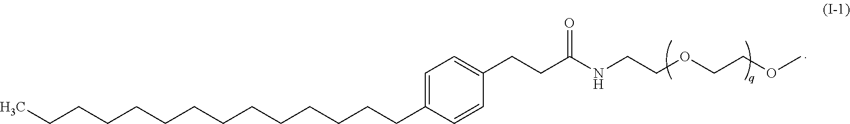

4. The compound of claim 1, wherein the compound is a compound of Formula (I); A is C₆₋₁₀ aryl; each R^{1A} is C₁₃₋₁₀₀ alkyl; L¹ is —(C═O)N(R^{N})—; and L² is

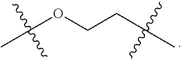 [SMILES: *CCO*]

5. The compound of claim 1, wherein the compound is a compound of Formula (I) and A is C₆₋₁₀ aryl.

6. The compound of claim 1, wherein the compound is a compound of Formula (I) and A is phenyl.

7. The compound of claim 1, wherein the compound is a compound of Formula (I) and at least one R^{1A} is C₁₃₋₁₀₀ alkyl.

8. The compound of claim 1, wherein the compound is a compound of Formula (I) and at least one R^{1A} is C₁₃₋₄₀ alkyl.

9. The compound of claim 1, wherein the compound is a compound of Formula (I) and at least one R^{1A} is C₁₃₋₂₀ alkyl.

10. The compound of claim 1, wherein the compound is a compound of Formula (I) and at least one R^{1A} is C₁₄ alkyl.

11. The compound of claim 1, wherein the compound is a compound of Formula (I) and at least one R^{1A} is C₁₃₋₁₀₀ alkenyl.

12. The compound of claim 1, wherein the compound is a compound of Formula (I) and at least one R^{1A} is C₁₃₋₁₀₀ alkynyl.

13. The compound of claim 1, wherein the compound is a compound of Formula (I) and L¹ is —(C═O)NH—.

14. The compound of claim 1, wherein the compound is a compound of Formula (I) and L² is selected from

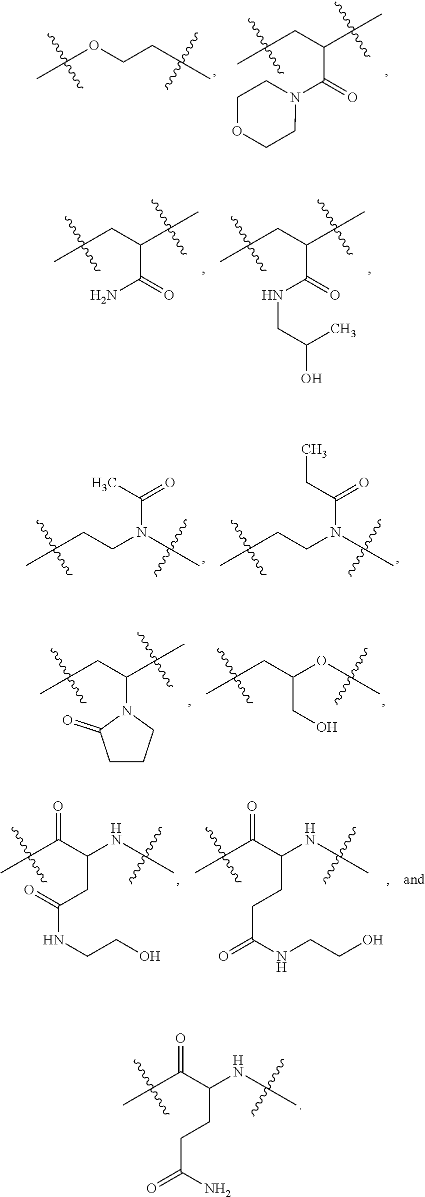

15. The compound of claim 1, wherein the compound is a compound of Formula (I) and L² is

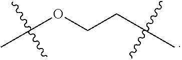 [SMILES: *CCO*]

16. The compound of claim 1, wherein the compound is a compound of Formula (I) and R² is H.

17. The compound of claim 1, wherein the compound is a compound of Formula (I) and R² is C₁₋₁₅ alkyl.

18. The compound of claim 1, wherein the compound is a compound of Formula (I) and R² is —OR^{O}.

19. The compound of claim 1, wherein the compound is a compound of Formula (I) and R² is —OCH₃.

20. The compound of claim 1, wherein the compound is a compound of Formula (I) and R² is a targeting ligand.

21. The compound of claim 20, wherein the targeting ligand is selected from a protein, a monosaccharide, a polysaccharide, a peptide, an aptamer, a small molecule, and a nucleic acid-based ligand.

22. The compound of claim 20, wherein the targeting ligand is selected from galactose and N-acetylgalactosamine (GalNAc).

23. The compound of claim 1, wherein the compound is a compound of Formula (I) and m is 1.

24. The compound of claim 1, wherein the compound is a compound of Formula (I) and n is 2.

25. The compound of claim 1, wherein the compound is a compound of Formula (I) and p is 2.

26. A compound of Formula (II): 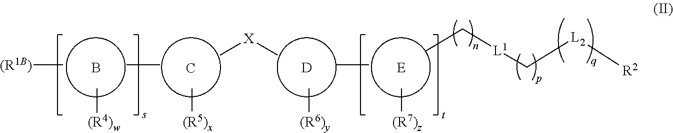
wherein:
X is selected from C(R³)₂, NR³, O, S, and 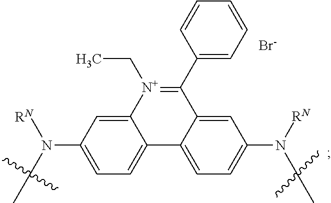
each R^{1B} is selected from C₁₋₁₀₀ alkyl, C₂₋₁₀₀ alkenyl, C₂₋₁₀₀ alkynyl, and C₁₋₁₀₀ haloalkyl, wherein the C₁₋₁₀₀ alkyl, C₁₋₁₀₀ alkenyl, and C₂₋₁₀₀ alkynyl forming R¹ is optionally substituted with one or more substituents independently selected from the group consisting of halo, —CN, —OR^{O}, —N(R^{N})₂, —(C═O)R^{N}, —(C═O)OR^{O}, —(C═O)N(R^{N})₂, —NR^{N}(C═O)R⁸, and —O(C═O)R⁸;
L¹ is selected from bond, —N(R^{N})—, —O—, —(C═O)—, —(C═O)O—, —(C═O)N(R^{N})—, —NR^{N}(C═O)—, and —O(C═O)—;
L² is selected from: 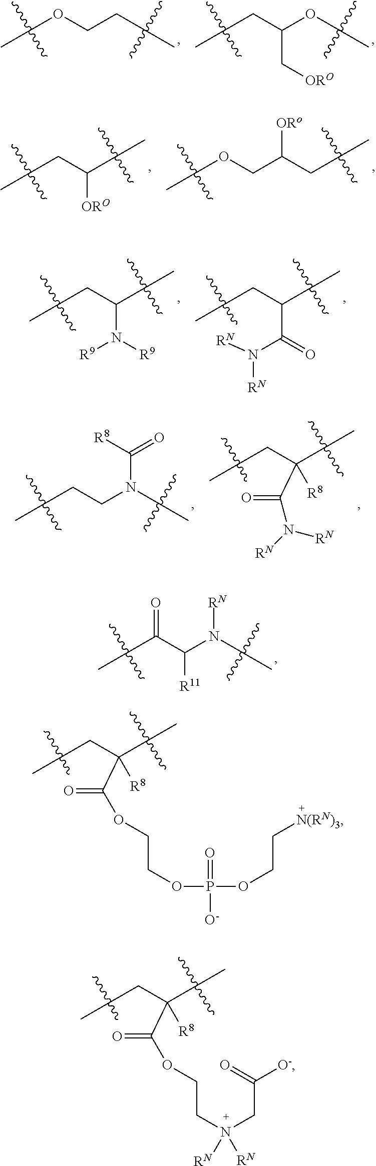
heparin, dextran, and chitosan;
R² is selected from H, C₁₋₁₅ alkyl, C₂₋₁₅ alkenyl, C₂₋₁₅ alkynyl, —OR^{O}, —(C═O)OR^{O}, —N(R^{N})₂, —N₃, 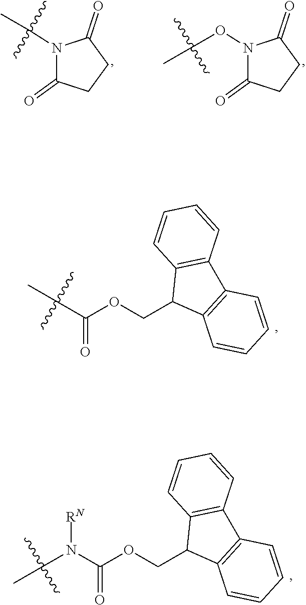
and targeting ligand;
each R³ is independently selected from H, C₁₋₁₅ alkyl, C₂₋₁₅ alkenyl, and C₂₋₁₅ alkynyl, C₃₋₁₀ cycloalkyl, C₆₋₁₀ aryl, 5- to 10-membered heteroaryl, and 4- to 10-membered heterocycloalkyl optionally substituted with one or more R¹⁰;
each Ring B, Ring C, Ring D, and Ring E is independently selected from C₆₋₁₀ aryl or 5- to 10-membered heteroaryl;
each R⁴, R⁵, R⁶, and R⁷ is independently selected from C₁₋₁₅ alkyl, C₂₋₁₅ alkenyl, C₂₋₁₅ alkynyl, C₃₋₁₀ cycloalkyl, C₆₋₁₀ aryl, 5- to 10-membered heteroaryl, 4- to 10-membered heterocycloalkyl, halo, —CN, —OR^{O}, —N(R^{N})₂, —(C═O)R^{N}, —(C═O)OR^{O}, —(C═O)N(R^{N})₂, —NR^{N}(C═O)R⁸, and —O(C═O)R⁸;
or an R⁵ and an R⁶, together with the atoms to which they are attached, come together to form C₆₋₁₀ aryl or 5- to 10-membered heteroaryl, wherein the C₆₋₁₀ aryl or 5- to 10-membered heteroaryl is optionally substituted with one or more R⁸;
each R⁸ is independently selected from H, C₁₋₁₅ alkyl, C₂₋₁₅ alkenyl, and C₂₋₁₅ alkynyl;
each R⁹ is independently selected from H, C₁₋₁₅ alkyl, C₂₋₁₅ alkenyl, and C₂₋₁₅ alkynyl;
or two R⁹, together with the N atom to which they are attached, come together to form 4- to 10-membered heterocycloalkyl optionally substituted with one or more oxo;
each R¹⁰ is independently selected from the group consisting of C₁₋₁₀₀ alkyl, C₂₋₁₀₀ alkenyl, C₂₋₁₀₀ alkynyl, C₁₋₁₀₀ haloalkyl, halo, —CN, —OR^{O}, oxo, —N(R^{N})₂, —(C═O)R^{N}, —(C═O)OR^{O}, —(C═O)N(R^{N})₂, —NR^{N}(C═O)R⁸, and —O(C═O)R⁸;
or an R⁵ and an R¹⁰, together with the atoms to which they are attached, come together to form C₆₋₁₀ aryl or 5- to 10-membered heteroaryl, wherein the C₆₋₁₀ aryl or 5- to 10-membered heteroaryl is optionally substituted with one or more R⁸;
or an R⁶ and an R¹⁰, together with the atoms to which they are attached, come together to form C₆₋₁₀ aryl or 5- to 10-membered heteroaryl, wherein the C₆₋₁₀ aryl or 5- to 10-membered heteroaryl is optionally substituted with one or more R⁸;
each R¹¹ is independently selected from C₁₋₁₅ alkyl, C₂₋₁₅ alkenyl, and C₂₋₁₅ alkynyl, optionally substituted with one or more R¹²;
each R¹² is independently selected from C₃₋₁₀ cycloalkyl, C₆₋₁₀ aryl, 5- to 10-membered heteroaryl, 4- to 10-membered heterocycloalkyl, halo, —CN, —OR^{O}, —N(R^{N})₂, —(C═O)R^{N}, —(C═O)OR^{O}, —(C═O)N(R^{N})₂, —NR^{N}(C═O)R⁸, —NR^{N}(C═NR^{N})R^{N}, —O(C═O)R⁸, and —SR⁸, wherein the C₃₋₁₀ cycloalkyl, C₆₋₁₀ aryl, 5- to 10-membered heteroaryl, 4- to 10-membered heterocycloalkyl is optionally substituted with one or more C₁₋₁₅ alkyl, C₂₋₁₅ alkenyl, and C₂₋₁₅ alkynyl, halo, —CN, —OR^{O}, and —N(R^{N})₂;
each R^{N} is independently selected from H, C₁₋₁₅ alkyl, C₂₋₁₅ alkenyl, and C₂₋₁₅ alkynyl, wherein the C₁₋₁₅ alkyl, C₂₋₁₅ alkenyl, and C₂₋₁₅ alkynyl forming R^{N} is optionally substituted with one or more substituents independently selected from the group consisting of halo, —CN, and —OR^{O};
each R^{O} is independently selected from H, C₁₋₁₅ alkyl, C₂₋₁₅ alkenyl, and C₂₋₁₅ alkynyl, wherein the C₁₋₁₅ alkyl, C₂₋₁₅ alkenyl, and C₂₋₁₅ alkynyl forming R^{O} is optionally substituted with one or more substituents independently selected from the group consisting of halo and —CN;
m is an integer selected from 1, 2, 3, 4, and 5;
n and p are each an integer independently selected from 0, 1, 2, 3, and 4;
q is an integer selected from 1 to 2,500;
s and t are each an integer independently selected from 0, 1, 2, 3, 4, and 5; and
w, x, y, and z are each an integer independently selected from 0, 1, 2, 3, and 4.

27. The compound of claim 26, wherein the compound is selected from:

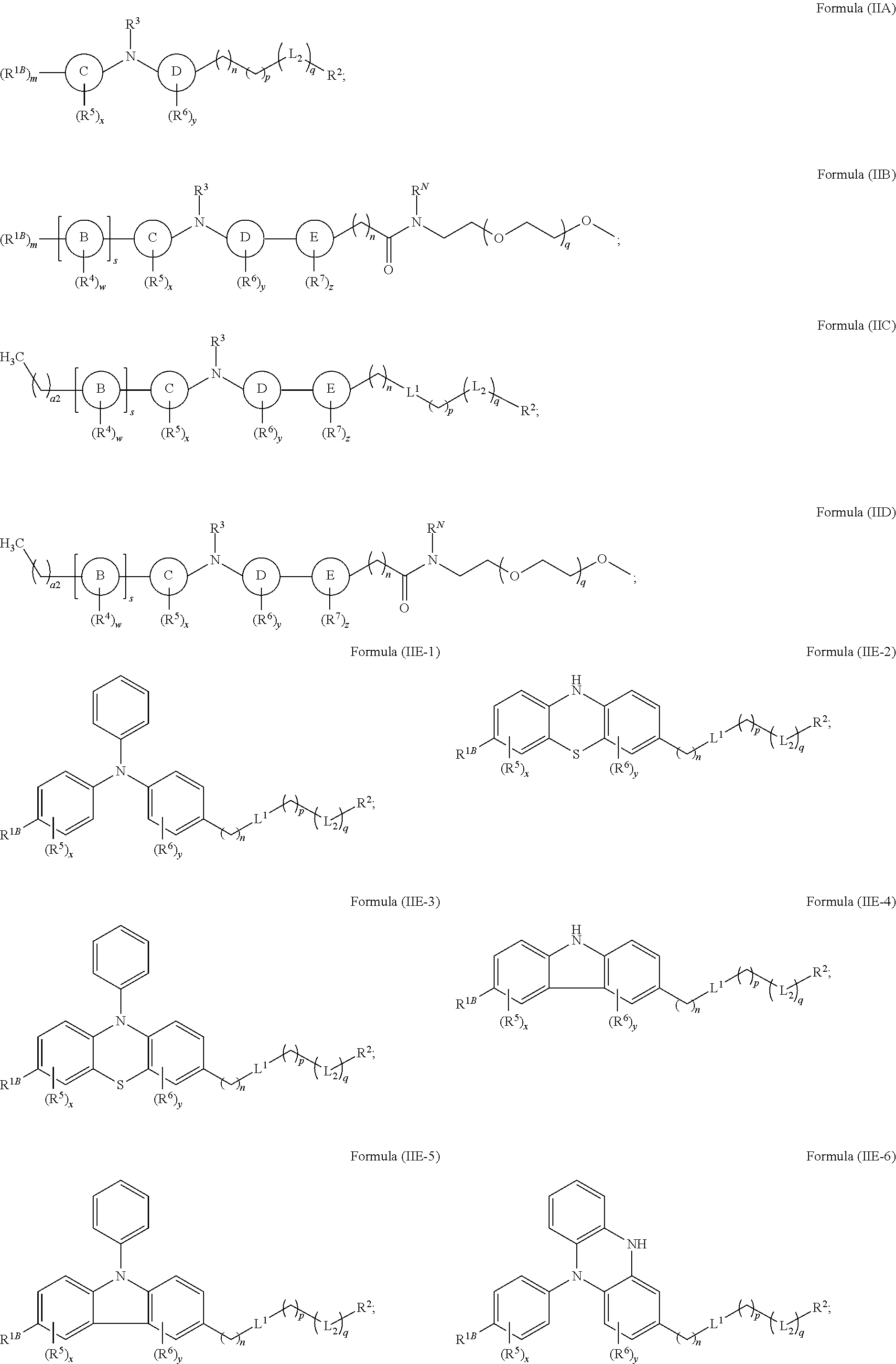

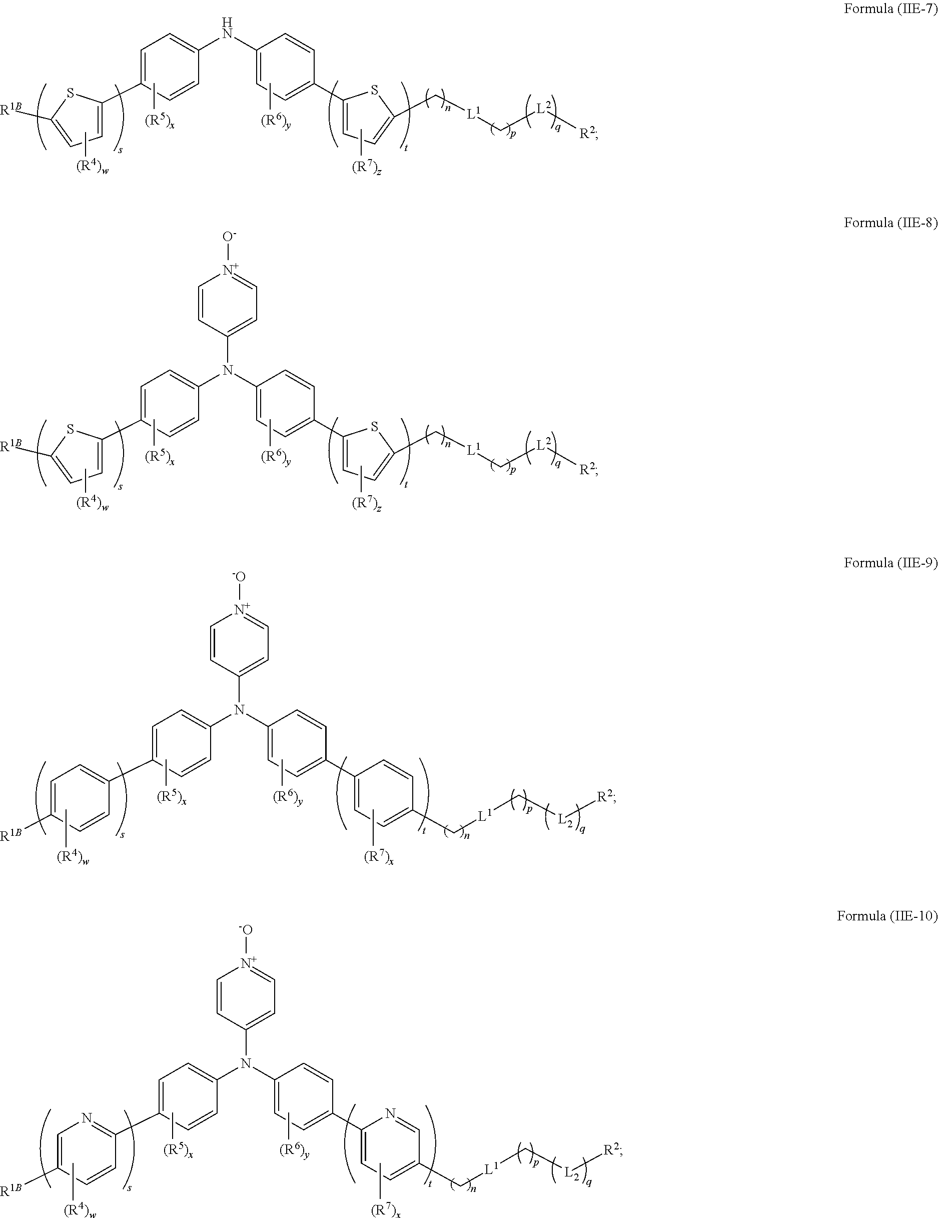

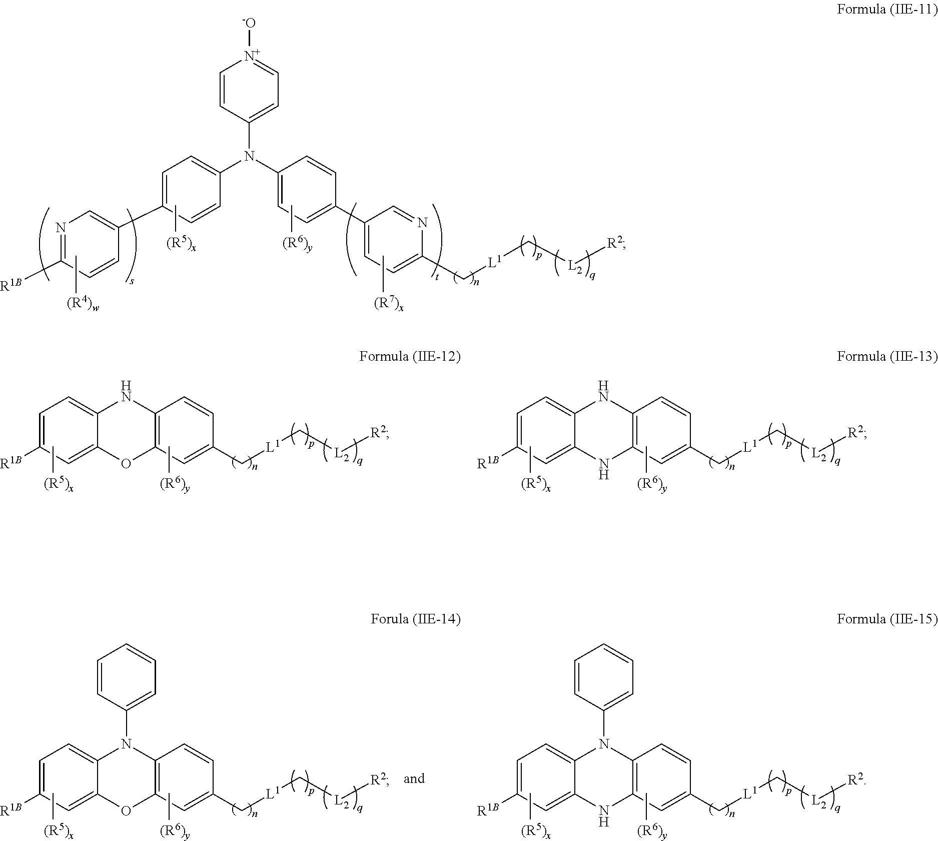

28. The compound of claim 26, wherein the compound is selected from:

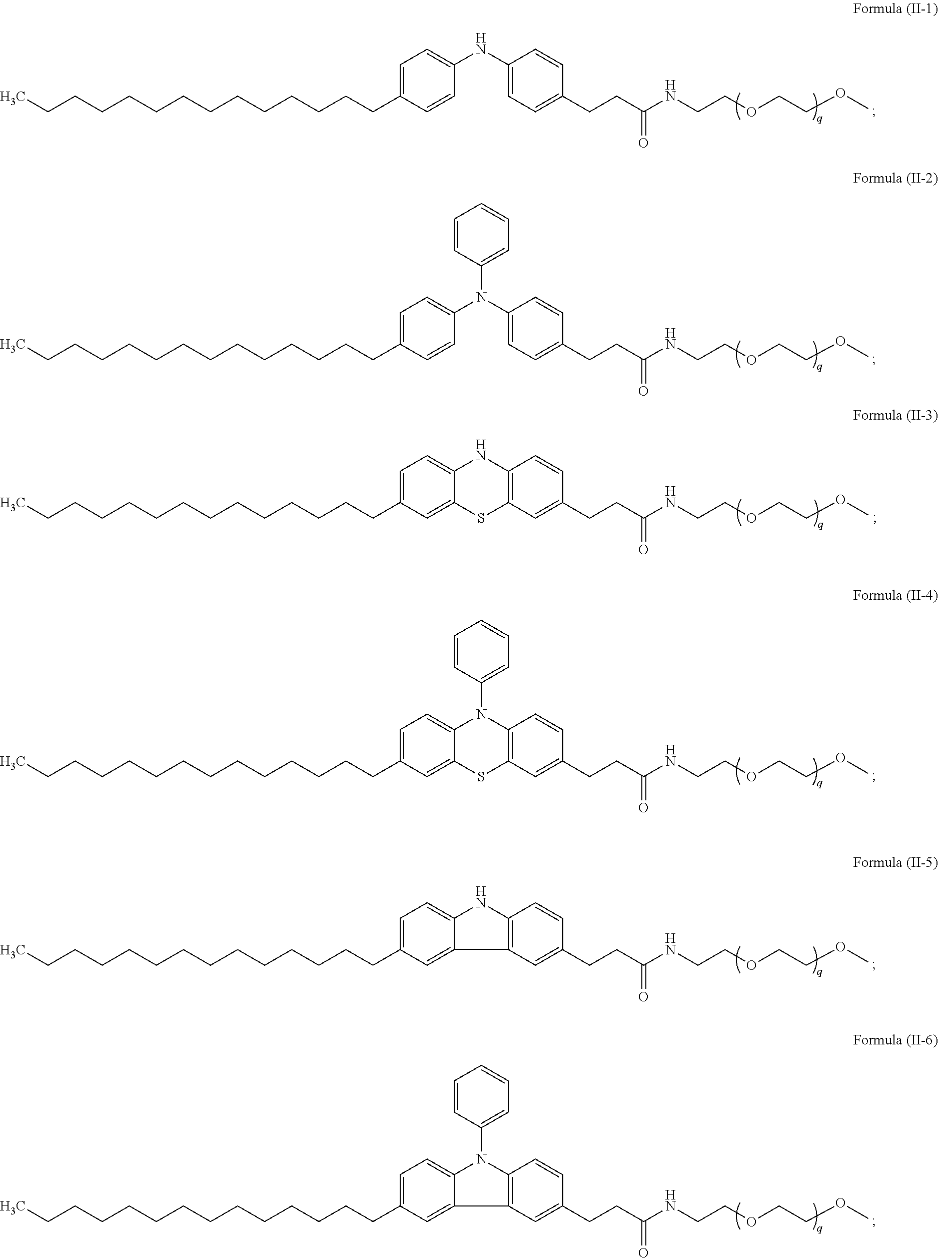

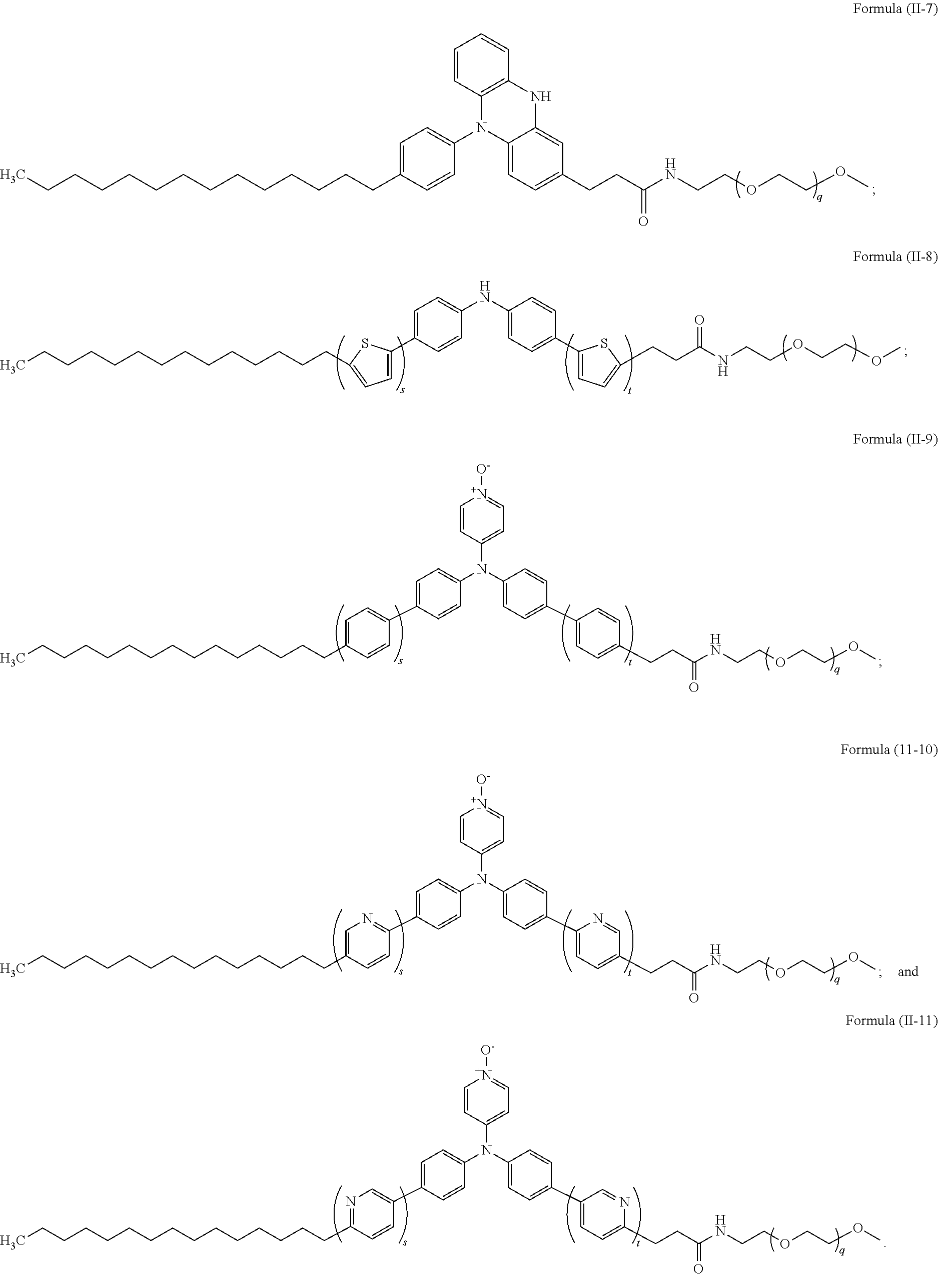

29. The compound of claim 26, wherein the compound is a compound of Formula (II); each R^{1B} is C₁₋₁₀₀ alkyl; L¹ is —(C═O)N(R^{N})—; L² is 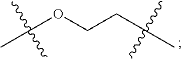 [SMILES: *CCO*]
R² is C₁₋₁₅ alkyl; R³ is selected from H and C₆₋₁₀ aryl; each R^{N} is H; and each R^{O} is C₁₋₁₅ alkyl.

30. The compound of claim 26, wherein the compound is a compound of Formula (II) and at least one R^{1B} is C₁₋₁₀₀ alkyl.

31. The compound of claim 26, wherein the compound is a compound of Formula (II) and at least one R^{1B} is C₁₋₄₀ alkyl.

32. The compound of claim 26, wherein the compound is a compound of Formula (II) and at least one R^{1B} is C₁₃₋₂₀ alkyl.

33. The compound of claim 26, wherein the compound is a compound of Formula (II) and at least one R^{1B} is C₁₄ alkyl.

34. The compound of claim 26, wherein the compound is a compound of Formula (II) and L¹ is —(C═O)NH—.

35. The compound of claim 26, wherein the compound is a compound of Formula (I) and L² is selected from

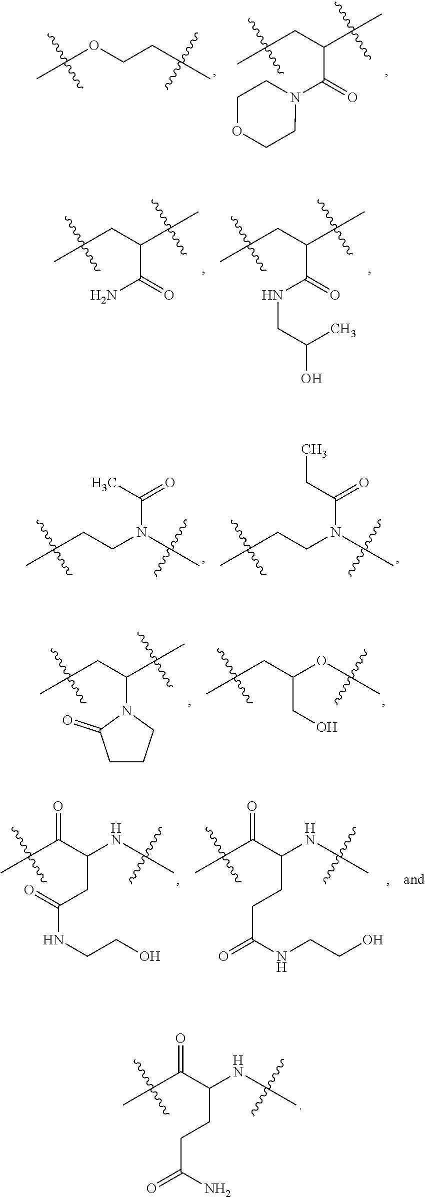

36. The compound of claim 26, wherein the compound is a compound of Formula (II) and L² is

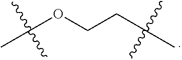 [SMILES: *CCO*]

37. The compound of claim 26, wherein the compound is a compound of Formula (II) and R² is H.

38. The compound of claim 26, wherein the compound is a compound of Formula (II) and R² is C₁₋₁₅ alkyl.

39. The compound of claim 26, wherein the compound is a compound of Formula (II) and R² is —OR^{O}.

40. The compound of claim 26, wherein the compound is a compound of Formula (II) and R² is a targeting ligand.

41. The compound of claim 40, wherein the targeting ligand is selected from a protein, a monosaccharide, a polysaccharide, a peptide, an aptamer, a small molecule, and a nucleic acid-based ligand.

42. The compound of claim 40, wherein the targeting ligand is selected from galactose and N-acetylgalactosamine (GalNAc).

43. The compound of claim 26, wherein the compound is a compound of Formula (II) and R³ is H.

44. The compound of claim 26, wherein the compound is a compound of Formula (II) and R³ is C₆₋₁₀ aryl.

45. The compound of claim 26, wherein the compound is a compound of Formula (II) and R³ is 5- to 10-membered heteroaryl.

46. The compound of claim 26, wherein the compound is a compound of Formula (II) and R³ is selected from H, phenyl, pyridinyl,

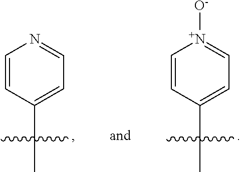 [SMILES: *c1ccncc1 ; *c1cc[n+]([O-])cc1]

47. The compound of claim 26, wherein the compound is a compound of Formula (II) and at least one Ring B is 5- to 10-membered heteroaryl.

48. The compound of claim 26, wherein the compound is a compound of Formula (II) and at least one Ring B is phenyl.

49. The compound of claim 26, wherein the compound is a compound of Formula (II) and at least one Ring B is pyridinyl.

50. The compound of claim 26, wherein the compound is a compound of Formula (II) and at least one Ring B is thiophenyl.

51. The compound of claim 26, wherein the compound is a compound of Formula (II) and Ring C is C₆₋₁₀ aryl.

52. The compound of claim 26, wherein the compound is a compound of Formula (II) and Ring C is phenyl.

53. The compound of claim 26, wherein the compound is a compound of Formula (II) and Ring D is C₆₋₁₀ aryl.

54. The compound of claim 26, wherein the compound is a compound of Formula (II) and Ring D is phenyl.

55. The compound of claim 26, wherein the compound is a compound of Formula (II) and at least one Ring E is 5- to 10-membered heteroaryl.

56. The compound of claim 26, wherein the compound is a compound of Formula (II) and at least one Ring E is phenyl.

57. The compound of claim 26, wherein the compound is a compound of Formula (II) and at least one Ring E is pyridinyl.

58. The compound of claim 26, wherein the compound is a compound of Formula (II) and at least one Ring E is thiophenyl.

59. The compound of claim 26, wherein the compound is a compound of Formula (II) and m is 1.

60. The compound of claim 26, wherein the compound is a compound of Formula (II) and n is 2.

61. The compound of claim 26, wherein the compound is a compound of Formula (II) and p is 2.

62. A composition comprising the compound of claims 1-55, optionally wherein the compound of Formula (I) or (II).

63. The composition of claim 62, wherein the composition is a nanoparticle, optionally a liposome.

64. The composition of claim 62, wherein the composition further comprises one or more additional lipids.

65. The composition of claim 64, wherein the additional lipids comprise:
one or more ionizable lipids, optionally selected from G0-Cm, DLin-MC3-DMA ((6Z,9Z,28Z,31Z)-heptatriacont-6,9,28,31-tetraene-19-yl 4-(dimethylamino)butanoate), SM-102, ALC-0315, and multi-tailed ionizable phospholipids (optionally iPhos); one or more phospholipids selected from phosphatidylethanolamine (optionally DOPE) and phosphatidylcholine (optionally DSPC); one or more cholesterol or analogue thereof; and/or other lipids, optionally selected from dioleoyl-3-trimethylammonium propane (DOTAP), 1,2-di-O-octadecenyl-3-trimethylammonium propane (DOTMA), DDAB, DODAP, EPC, and 18BMP.

66. The composition of claim 63, wherein the nanoparticle further comprises a cargo.

67. The composition of claim 66, wherein a cargo is presented on the surface of the nanoparticle, or wherein the nanoparticle comprises a core and an envelope, wherein the core comprises a lipid and a cargo, optionally wherein the cargo is complexed with the lipid.

68. The composition of claim 66, wherein the cargo comprises RNA, DNA, protein, or a small molecule.

69. The composition of claim 68, wherein the RNA or DNA encodes, or the protein comprises: a therapeutic protein, a tumor suppressor, an antigen, a cytokine, or a co-stimulatory molecule.

70. The composition of claim 69, wherein the therapeutic protein is listed in Table 2, 4, or 6.

71. The composition of claim 69, wherein the tumor suppressor is listed in Table 5.

72. The composition of claim 68, wherein the mRNA comprises one or more modifications, preferably selected from the group consisting of ARCA capping; enzymatic polyadenylation to add a tail of 100-250 adenosine residues; and substitution of one or both of cytidine with 5-methylcytidine and/or uridine with pseudouridine.

73. A method of treating a subject who has cancer, the method comprising administering to the subject a therapeutically effective amount of the composition of claim 68, wherein the RNA or DNA encodes, or the protein comprises: a tumor suppressor, an antigen, a cytokine, or a co-stimulatory molecule.

74. A method of treating a subject who has a genetic disorder, the method comprising administering to the subject a therapeutically effective amount of the composition of claim 68, wherein the RNA or DNA encodes, or the protein comprises, a therapeutic for the genetic disorder.

75. A method of treating a subject who has hemophilia, the method comprising administering to the subject a therapeutically effective amount of the composition of claim 68, wherein the RNA or DNA encodes, or the protein comprises, Factor VIII or Factor IX.

76. A method of treating a subject who has an infectious disease associated with an infectious agent, or reducing risk of developing an infectious disease with an infectious agent, the method comprising administering to the subject a therapeutically effective amount of the composition of claim 68, wherein the RNA or DNA encodes, or the protein comprises an antigen associated with the infectious agent.

77. A method of administering a therapeutic agent to a subject, the method comprising administering to the subject a therapeutically effective amount of the composition of claim 68, wherein the cargo comprises the therapeutic agent, or comprises RNA or DNA that encodes, or a protein that comprises, the therapeutic agent.

78. The method of claim 77, wherein the therapeutic agent is an antibody or a gene editing reagent.
